(19) United States
(12) Patent Application Publication
(10) Pub. No.: US 2005/0112098 A1
(43) Pub. Date: May 26, 2005

(54) Pseudotyped viruses and methods for their use

(75) Inventors: Paul B. McCray JR., Iowa City, IA (US); David A. Sanders, West Lafayette, IN (US); Scott A. Jeffers, Aurora, CO (US); Beverly L. Davidson, North Liberty, IA (US); Patrick L. Sinn, Iowa City, IA (US)

Correspondence Address: MUETING, RAASCH & GEBHARDT, P.A., P.O. BOX 581415, MINNEAPOLIS, MN, 55458, US

(73) Assignees: Purdue Research Foundation, West Lafayette, IN (US); University of Iowa Research Foundation, Iowa City, IA (US)

(21) Appl. No.: 10/811,353

(22) Filed: Mar. 26, 2004

Related U.S. Application Data
(63) Continuation-in-part of application No. PCT/US03/17577, filed on Jun. 4, 2003.
(63) Continuation-in-part of application No. PCT/US02/34545, filed on Oct. 28, 2002.
(60) Provisional application No. 60/386,064, filed on Jun. 4, 2002.
(60) Provisional application No. 60/458,070, filed on Mar. 27, 2003.
(60) Provisional application No. 60/458,070, filed on Mar. 27, 2003.
(60) Provisional application No. 60/353,221, filed on Oct. 26, 2001.
(60) Provisional application No. 60/356,436, filed on Oct. 26, 2001.

(51) Int. Cl. A61K 48/00; A61K 38/16

(52) U.S. Cl. 424/93.2; 514/44; 514/8

(57) ABSTRACT

The invention provides a method to introduce a selected nucleic acid sequence into an airway epithelial cell that involves contacting the airway epithelial cell with a pseudotyped retrovirus that includes a glycoprotein in which a portion of an O-glycosylation region within the glycoprotein has been deleted and a retroviral capsid that includes the selected nucleic acid sequence. The invention also provides a method to reduce or eliminate the symptoms of cystic fibrosis in a mammal that involves contacting an airway epithelial cell of the mammal with a pseudotyped retrovirus that includes a glycoprotein in which a portion of an O-glycosylation region within the glycoprotein has been deleted and a retroviral capsid that includes a nucleic acid sequence that encodes a cystic fibrosis transmembrane regulator protein.

### RELATED APPLICATIONS

This application claims priority to International Application No. PCT/US02/34545, filed Oct. 28, 2002, which claims the benefit of U.S. Provisional Application Ser. No. 60/353,221, filed Oct. 26, 2001 and 60/356,436, filed Oct. 26, 2001; and further claims priority to International Application No. PCT/US03/17577, filed Jun. 4, 2003, which claims the benefit of U.S. Provisional Application Ser. No. 60/386,064, filed Jun. 4, 2002 and 60/458,070 filed Mar. 27, 2003; and further claims the benefit of U.S. Provisional Patent Application Ser. No. 60/458,070, filed Mar. 27, 2003. Each of these patent applications is incorporated herein by reference in its entirety.

### STATEMENT OF GOVERNMENT RIGHTS

The invention described herein was developed with support from the National Institutes of Health under Grant Numbers HL-61460, HL-51670 and NS-34568; and the National Research Service under Grant Number HL-67623. The U.S. Government has certain rights in the invention.

### BACKGROUND OF THE INVENTION

Gene therapy is one of the fastest growing areas in experimental medicine. However most studies are only Phase I or Phase II clinical studies designed mainly to evaluate the toxicity of the viral vectors and constructs being used. A major drawback has been the design of vectors that are both safe and efficacious.

Viral vectors offer promise for use in the delivery of genes and other nucleic acids to cells for gene therapy. Recently, retroviruses have generated a great deal of interest for use as viral vectors.

Retroviruses are ribonucleic acid (RNA) viruses that include an RNA genome enclosed within a viral capsid wherein the capsid is surrounded by an envelope, or lipid bilayer. Glycoproteins present in the lipid bilayer interact with receptors on the surface of various host cells and allow the retroviruses to enter the host cell. Once in the cell, the retroviruses reverse transcribe the RNA of the viral genome into a double-stranded DNA and incorporate the DNA into the cellular genome as a provirus. Gene products from the integrated foreign DNA may then be produced so that progeny viral particles may be assembled. As retroviruses can be modified to carry exogenous nucleotide sequences of interest, such recombinant retroviruses have a variety of uses. For example, such recombinant retroviruses are important in introducing desired exogenous sequences into a cell, so that relatively high levels of the protein encoded by the sequences may be produced. However, use of such recombinant retroviruses has several drawbacks.

One drawback of retroviruses is that they do not have a broad host range. Efforts at increasing the host range of retroviruses have included substituting the envelope glycoprotein of the retrovirus with that of a different virus, thus forming a pseudotyped retrovirus. The pseudotyped retrovirus advantageously has the host range of the virus from which the glycoprotein was derived. However, some retroviruses have been pseudotyped with viral glycoproteins that are toxic to cells. This causes the packaging cells to only produce the pseudotyped virus for a limited time. Furthermore, in many cases, the pseudotyped retrovirus cannot be stably produced and may not be produced at a high titer.

Another drawback of retroviral vectors is their inability to transduce non-dividing cells, such as airway epithelium, hepatocytes and brain glial cells. Retroviral vectors used in ex vivo and in vivo transduction of hepatocytes required inducing the hepatocytes to proliferate by complex and artificial procedures.

One clinical trial was conducted to treat familial hypercholesterolemia by retroviral mediated ex vivo gene transfer. The LDL receptor gene was introduced into hepatocytes that had been surgically removed from patients and which were then reinfused into the liver following gene transduction. There was no convincing evidence, however, of therapeutic efficacy. Liver biopsies were removed after treatment, and few cells tested positive for the expression of LDL-receptor, indicating low transduction efficiency. In vivo retroviral-mediated transduction of hepatocytes was even more complicated, as it required artificial regeneration of the liver to give dividing cells (Ferry et al., Hum. Gene Ther., 9:1975 (1998)).

Despite certain drawbacks, viral vectors offer several potential advantages for attaining persistent expression of a therapeutic gene in numerous tissues such as those found in the brain, lung and liver. For example, within one year of the 1989 discovery of the cystic fibrosis transmembrane conductance regulator (CFTR) gene and the first identification of disease-associated mutations (Kerem et al., Science, 245:1073 (1989)), in vitro complementation experiments demonstrated the feasibility of gene transfer as means to correct the physiologic defect in chloride transport associated with cystic fibrosis (Drumm et al., Cell, 62:1227 (1990) and Rich et al., Nature, 347:358 (1990)). Subsequently, a tremendous effort has been directed towards the practical application of gene transfer as a new therapeutic approach to treat CF-associated lung disease. Its practical application has yet to be achieved despite the tremendous promise and appeal of this approach. Thus, use of a viral vector to transfer a gene to airway epithelial cells to treat a disease such as cystic fibrosis would be very desirable.

The airway epithelium possesses several unique properties that make it a formidable target for successful gene transfer. Among these are the many innate and adaptive host defense functions that the epithelium and resident immune effecter cells perform. The pulmonary epithelium has evolved to prevent the invasion of the host by microbes and these same strategies may act as barriers for gene transfer vectors.

Recombinant vectors based on Moloney murine leukemia virus (Mo-MLV or MMLV) were the first retroviruses used for gene transfer to airway epithelium. Several studies demonstrated the potential for Mo-MLV-based vectors to persistently transduce airway epithelium by showing that the retroviral vectors could transduce airway epithelium ex vivo and furthermore, that the Cl-transport defect in cystic fibrosis (CF) airway epithelial cells was corrected by transducing the cells in vitro with a Mo-MLV retrovirus vector expressing the CFTR cDNA. However, Mo-MLV-based vectors require cell division in order for the integration complex to enter the nucleus and the normal airway epithelium is mitotically quiescent with less than 1% of the cells dividing. Therefore, transduction efficiency is low in airway epithelial cells.

Accordingly, it would be desirable to have a viral vector, such as a retroviral vector, that can efficiently transduce non-dividing cells that include hepatocytes, brain glial cells and airway epithelial cells. It would be further desirable if such vectors were efficient in transducing cells, such as hepatocytes, brain glial, and airway epithelial cells, in vivo.

### SUMMARY OF THE INVENTION

The invention provides a method to introduce a selected nucleic acid sequence into an airway epithelial cell that involves contacting the airway epithelial cell with a pseudotyped retrovirus that includes a glycoprotein in which a portion of an O-glycosylation region within the glycoprotein has been deleted and a retroviral capsid that includes the selected nucleic acid sequence.

The glycoprotein can have reduced glycosylation when compared to glycosylation of a glycoprotein in which a portion of the O-glycosylation region has not been deleted. In some examples, the glycoprotein can be a filoviral glycoprotein. Preferably, the glycoprotein is from a Marburg virus or an Ebola virus. In one example, the Ebola virus glycoprotein lacks amino acids 309-489.

The retroviral capsid can be obtained from any of many retroviruses. In one example, the retroviral capsid is a lentiviral capsid. Preferably, the lentiviral capsid is from a feline immunodeficiency virus.

The airway epithelial cell can be a mammalian airway epithelial cell. Preferably, the airway epithelial cell is a human airway epithelial cell.

A pseudotyped retrovirus can be contacted with the airway epithelial cell in vivo, in vitro or ex vivo. In some examples, the airway epithelial cell is contacted with the pseudotyped retrovirus on the basolateral surface of the airway epithelial cell. In other examples, the airway epithelial cell is contacted with the pseudotyped retrovirus on the apical surface of the airway epithelia cell.

The pseudotyped retrovirus can be included within a composition. In some examples, the composition is a pharmaceutical composition. The composition can include an agent that disrupts junctions between cells. In some examples, the agent can be EGTA, EDTA or sodium citrate.

The method can futher include the step of contacting an airway epithelial cell with an agent that disrupts junctions between cells. The airway epithelial cell can be contacted with the agent before, during or after the airway epithelial cell is contacted with the pseudotyped retrovirus. In some examples, the agent that disrupts junctions between cells is EGTA, EDTA or sodium citrate.

In some examples, a selected nucleic acid sequence is present within an airway epithelial cell prior to being contacted with a pseudotyped retrovirus. In other examples, a selected nucleic acid sequence is not present within an airway epithelial cell prior to being contacted with a pseudotyped retrovirus. Preferably, the selected nucleic acid sequence encodes cystic fibrosis transmembrane regulator protein (CFTR).

The invention provides a method to reduce or eliminate the symptoms of cystic fibrosis in a mammal that involves contacting an airway epithelial cell of the mammal with a pseudotyped retrovirus that includes a glycoprotein in which a portion of an O-glycosylation region within the glycoprotein has been deleted and a retroviral capsid that includes a nucleic acid sequence that encodes a cystic fibrosis transmembrane regulator protein.

In some examples, the glycoprotein has reduced glycosylation when compared to glycosylation of a glycoprotein in which a portion of the O-glycosylation region has not been deleted. In some examples, the glycoprotein can be a filoviral glycoprotein. Preferably, the glycoprotein is from a Marburg virus or an Ebola virus. In one example, the Ebola virus glycoprotein lacks amino acids 309-489.

The retroviral capsid can be obtained from any of many retroviruses. In one example, the retroviral capsid is a lentiviral capsid. Preferably, the lentiviral capsid is from a feline immunodeficiency virus.

Any of many mammals can be used within the method. In one example, the mammal is a human.

In some examples, an airway epithelial cell is contacted with a pseudotyped retrovirus on the apical or basolateral side of the airway epithelial cell. Preferably, an airway epithelial cell is contacted with a pseudotyped retrovirus on the apical side of the airway epithelial cell.

The pseudotyped retrovirus can be included within a composition. In some examples, the composition is a pharmaceutical composition. The composition can include an agent that disrupts junctions between cells. In some examples, the agent can be EGTA, EDTA or sodium citrate.

The method can further include the step of contacting an airway epithelial cell with an agent that disrupts junctions between cells. The airway epithelial cell can be contacted with the agent before, during or after the airway epithelial cell is contacted with the psuedotyped retrovirus. In some examples, the agent that disrupts junctions between cells is EGTA, EDTA or sodium citrate.

### BRIEF DESCRIPTION OF THE DRAWINGS

FIG. 1 illustrates the steps involved in retroviral transduction of polarized epithelia.

FIG. 2 is a schematic representation of Ebola virus glycoprotein (GP). The GP₁ and GP₂ subunits of GP are drawn to scale (residue numbers are indicated below the diagram). The positions of the signal sequence (cross-hatching), conserved cysteine residues (S), the mucin-like region (region of O-linked glycosylation), the furin cleavage site, the fusion peptide (vertical lines), the coiled-coil domain (diagonal lines), and the membrane-spanning domain (horizontal line) are indicated.

FIG. 3 shows the analysis of the expression and incorporation into pseudotyped retroviruses of the Δ309-489 Ebola virus GP. The migration positions of the mature GP₁, GP₀ (the glycosylated but uncleaved form), GPₚᵣₑ (the N-glycosylated but not O-glycosylated uncleaved form), and deglycosylated GP₀ and GPₚᵣₑ forms of wild-type (WT) GP and of the GP₁, GP₀, and deglycosylated GP₁ and GP₀ forms of Δ309-489 GP are indicated. (Neg) ΦNX cells transfected with pcDNA3 vector.

FIG. 4 is an illustration showing a feline immunodeficiency virus (FIV) vector construct.

FIG. 5 shows the characterization of entry paths for respiratory pathogens (PIV3) parainfluenza 3 virus, (RSV) respiratory syncytial virus, (HCV) human coronavirus 229E, and (MV) measles virus in polarized human airway epithelia through the basal or apical surface of the cells.

FIG. 6 is a schematic showing the amino acid sequence of the Marburg glycoprotein (SEQ. ID. NO: 1). The amino acid positions are numerically indicated on the left.

FIG. 7 is a schematic showing mutations at the carboxyl-terminus of the amino acid sequences of the Marburg glycoprotein (SEQ. ID. NOs: 10-16; top to bottom)

FIG. 8 is a bar graph showing the effect of mutations at the carboxyl-terminus of the Marburg envelope glycoprotein on the titer of FIV pseudotyped with the mutant Marburg glycoproteins.

FIG. 9A is a photograph showing gene transfer in human airway epithelia that were exposed on the apical surface to FIV pseudotyped with vesicular stomatitis virus G protein (VSV-G).

FIG. 9B is a photograph showing gene transfer in human airway epithelia that were exposed on the basolateral surface to FIV pseudotyped with VSV-G.

FIG. 9C is a photograph showing gene transfer in human airway epithelia that were exposed on the apical surface to FIV pseudotyped with Marburg glycoprotein.

FIG. 9D is a photograph showing gene transfer in human airway epithelia that were exposed on the basolateral surface to FIV pseudotyped with Marburg glycoprotein.

FIG. 10A is a photograph of showing the production of β-galactosidase by astrocytes transduced with Ross River Virus (RRV)-pseudotyped FIV.

FIG. 10B is a photograph of showing the production of glial fibrillary acidic protein (GFAP) by astrocytes transduced with RRV-pseudotyped FIV.

FIG. 10C is a photograph of showing the production of β-galactosidase and GFAP by astrocytes transduced with RRV-pseudotyped FIV.

FIG. 11A is a photograph showing the production of β-galactosidase by oligodendrocytes transduced with RRV-pseudotyped FIV.

FIG. 11B is a photograph showing the production of 2′,3′-cyclic nucleotide-3′-phosphodiesterase (CNPase) by oligodendrocytes transduced with RRV-pseudotyped FIV.

FIG. 11C is a photograph showing the production of α-galactosidase and CNPase by oligodendrocytes transduced with RRV-pseudotyped FIV.

FIG. 12A is a photograph showing the production of β-galactosidase by oligodendrocytes transduced with RRV-pseudotyped FIV.

FIG. 12B is a photograph showing the production of CNPase by oligodendrocytes transduced with RRV-pseudotyped FIV.

FIG. 12C is a photograph showing the production of β-galactosidase and CNPase by oligodendrocytes transduced with RRV-pseudotyped FIV.

FIG. 13A is a photograph of the en face view of duplicate samples of X-gal stained liver slices showing the efficiency of transducing hepatocytes with RRV I pseudotyped FW having a β-galactosidase reporter gene.

FIG. 13B is a photograph of the en face view of duplicate samples of X-gal stained liver slices showing the efficiency of transducing hepatocytes with VSV-G pseudotyped FIV having a β-galactosidase reporter gene.

FIG. 13C is a photograph of the en face view of duplicate control samples of X-gal stained liver slices treated with phosphate buffered saline (PBS).

FIG. 13D is a photograph of a liver slice stained with hematoxylin and eosin I showing efficiency of transducing hepatocytes with RRV pseudotyped FIV having a β-galactosidase reporter gene.

FIG. 13E is a photograph of a liver slice stained with hematoxylin and eosin showing efficiency of transducing hepatocytes with VSV-G pseudotyped FIV having a β-galactosidase reporter gene.

FIG. 13F is a photograph of a control liver slice stained with hematoxylin and eosin that was treated with PBS.

FIG. 14A is a bar graph showing the effect of RRV pseudotyped FIV and VSV-G pseudotyped FIV on liver function as measured by serum glutamic pyruvic transaminase (SGPT) levels.

FIG. 14B is a bar graph showing the effect of RRV pseudotyped FIV and VSV-G pseudotyped FIV on liver function as measured by serum glutamic oxalacetic transaminase (SGOT) levels.

FIG. 15 is a radioimmunoprecipitation assay (RIPA) of Ebola virus glycoprotein (GP) in 293 cells. (M) immunoprecipitated GPs secreted or released into the medium; (L) immunoprecipitated GPs associated with the cell monolayer. The migration positions of GP₁, GP₂, and GPₚᵣₑ are indicated on the left with an arrow. Asterisks in the GP₁ region identify increased levels of this GP in the medium relative to cell-associated GP₁, compared to the levels in the wild type (WT). Asterisks in the GP₂ region identify faster-migrating forms of GP₂. (Neg) transfection of pTM1 (NcoI) vector into 293 cells infected with a recombinant vaccinia virus (vTF7-3) expressing T7 RNA polymerase.

FIG. 16 illustrates the migration of Ebola virus glycoprotein (GP) under nonreducing conditions. Shown is an autoradiogram of SDS-PAGE analysis (under nonreducing conditions) of wild-type (WT) GP and of the proteins analyzed in FIG. 15 that showed increased release of GP₁ into the medium. (M) immunoprecipitated GPs secreted or released into the medium. (L) immunoprecipitated GPs associated with the cell monolayer. The migration positions of GP₁, GP₂, and GPₚᵣₑ are indicated on the left with arrows.

FIG. 17 shows the analysis of the expression and incorporation into pseudotyped retroviruses of Ebola virus GPs with substitutions of ectodomain cysteine residues. The cell lysates (L) and viral particles collected from the culture medium (M) were analyzed by SDS-PAGE (8.5% acrylamide) and immunoblotting with anti-Ebola virus SGP-GP antibody. Analysis of a cell lysate aliquot that was treated with PNGase F (+), which removes N-linked glycosylation, is also shown. The migration positions of mature GP₁, GP₀ (the glycosylated but uncleaved form), GPₚᵣₑ (the N-glycosylated but not O-glycosylated uncleaved form), and deglycosylated GP₀ and GPₚᵣₑ are indicated. (WT) wild type; (Neg) ΦNX cells transfected with pcDNA3 vector.

FIG. 18 shows the analysis of the expression and incorporation into pseudotyped retroviruses of Ebola virus GPs with substitutions of membrane-spanning domain cysteine residues. The migration positions of mature GP₁, GP₀ (the glycosylated but uncleaved form), GPₚᵣₑ (the N-glycosylated but not O-glycosylated uncleaved form), and deglycosylated GP₀ and GPₚᵣₑ are indicated. (WT) wild type; (Neg) ΦNX cells transfected with pcDNA3 vector.

FIG. 19 shows the analysis of the expression and incorporation into pseudotyped retroviruses of Ebola virus GPs with substitutions eliminating sites of N-linked glycosylation. The migration positions of mature GP₁, GP₀ (the glycosylated but uncleaved form), GPₚᵣₑ (the N-glycosylated but not O-glycosylated uncleaved form), and GP₂ are indicated. (WT) wild type, (Neg) ΦNX cells transfected with pcDNA3 vector.

FIG. 20 shows the analysis of the glycosylation of the Δ309-489 Ebola virus GP incorporated into pseudotyped retroviruses. Samples treated with PNGase F, with a combination of PNGase F, sialidase A, and endo-O-glycosidase, or with a combination of the previous three enzymes and β(1-4)-galactosidase and glucosaminidase are indicated. The migration positions of the mature GP₁ forms of the wild-type (WT) and Δ309-489 GPs are indicated with arrows. (Neg) ΦNX cells transfected with pcDNA3 vector.

FIG. 21A shows a cysteine bridge model for Ebola virus GP and comparison of GP₂ to the Rous sarcoma virus GP transmembrane (TM) subunit. The positions of the signal sequence (cross-hatching), conserved cysteine residues (S), the mucin-like region (region of O-linked glycosylation), the furin cleavage site, the fusion peptide (vertical lines), the coiled-coil domain (diagonal lines), the membrane-spanning domain (horizontal line) and the N-glycosylation sites (Y) are indicated.

FIG. 21B illustrates a proposed cysteine bridge model for the transmembrane protein of Rous sarcoma virus is presented for comparison with that for Ebola virus GP₂.

FIG. 22A illustrates folate receptor alpha (FRα) expression in primary cultures of human airway epithelia that were immunohistochemically stained and viewed with a confocal microscopy from an en face section. Scale bar=50 μm.

FIG. 22B illustrates FRα expression in primary cultures of human airway epithelia that were immunohistochemically stained and viewed with a confocal microscopy from a vertical section.

FIG. 22C illustrates FRα expression in primary cultures of human airway epithelia that were immunohistochemically stained and viewed with a confocal microscopy from an en face section at high power. Scale bar=50 μm.

FIG. 22D illustrates FRα expression in primary cultures of human airway epithelia that were immunohistochemically stained and viewed with a confocal microscopy from a vertical section at high power.

FIG. 22E illustrates FRα expression in primary cultures of human airway epithelia that were immunohistochemically stained and viewed with a confocal microscopy from an en face section at low power. Scale bar=100 μm.

FIG. 22F shows staining with an isotype control primary antibody. Scale bar=50 μm.

FIG. 22G shows primary cultures of human airway cells imaged en face section following PI-PLC treatment to confirm enzyme function. Scale bar=50 μm.

FIG. 22H shows primary cultures of human airway cells imaged in vertical section following PI-PLC treatment to confirm enzyme function.

FIG. 221 shows a western blot using the FRα-specific monoclonal antibody followed by an anti-mouse alkaline phosphatase-conjugated secondary antibody. The expected 42-kDa band is indicated with an arrow. (hAEC) human airway epithelial cell; (hT) human trachea; (KB) KB cell line.

FIG. 23 shows transduction levels from three samples of polarized airway cell lines obtained from three independent human specimens with pseudotyped FIV vectors. (RLU) relative light units, (*) indicates that P<0.01, (EBO-FIV) feline immunodeficiency virus pseudotyped with Ebola virus glycoprotein, (EBOΔO-FIV) feline immunodeficiency virus pseudotyped with Ebola virus glycoprotein deletion mutant, (VSVG-FIV) feline immunodeficiency virus pseudotyped with vesicular stomatitis virus G protein.

FIG. 24A shows transduction levels of three samples of polarized airway cell lines obtained from three independent human specimens with pseudotyped FIV vector following pretreatment with an IgG1 isotype antibody (black bars) or an FRα-blocking antibody (gray bars). (RLU) relative light units, (*) indicates P<0.01, (EBOΔO-FIV) feline immunodeficiency virus pseudotyped with Ebola virus glycoprotein deletion mutant, (VSVG-FIV) feline immunodeficiency virus pseudotyped with vesicular stomatitis virus G protein.

FIG. 24B shows transduction levels of three samples of polarized airway cell lines obtained from three independent human specimens with pseudotyped FIV vector following pretreatment with PI-PLC (gray bars) or without PI-PLC (black bars). (RLU) relative light units, (*) indicates P<0.01, (EBOΔO-FIV) feline immunodeficiency virus pseudotyped with Ebola virus glycoprotein deletion mutant, (VSVG-FIV) feline immunodeficiency virus pseudotyped with vesicular stomatitis virus G protein.

FIG. 25A shows relative FRα mRNA levels in immortalized cell lines (KB, HT1080, HOS, A549, H441, HBE and IB3) from which total mRNA was purified and analyzed by ribonuclease protection assay (RPA) by using FRα and human actin (hACT)-specific [α-³²P]UTP-labeled antisense probes. FRα expression was normalized to actin expression.

FIG. 25B shows relative FRα protein abundance in protein lysates from the indicated cell lines (KB, HT1080, HOS, A549, H441, HBE and IB3) that were incubated with either an FRα-specific monoclonal antibody (open curve) or an isotype control (shaded curve) followed by incubation with an anti-mouse FITC-conjugated secondary antibody and FACS analysis.

FIG. 26A shows relative transduction levels of immortalized cell lines (KB, HT1080, HOS, A549, H441, HBE and IB3) transduced with EBO-FIV, EBOΔO-FIV or VSG-FIV. (EBO-FIV) feline immunodeficiency virus pseudotyped with Ebola virus glycoprotein, (EBOΔO-FIV) feline immunodeficiency virus pseudotyped with Ebola virus glycoprotein deletion mutant, (VSVG-FIV) feline immunodeficiency virus pseudotyped with vesicular stomatitis virus G protein. (RLU) relative light units, (*) indicates that P<0.01.

FIG. 26B shows relative transduction levels of immortalized cell lines (KB, HT1080, HOS, A549, H441, HBE and IB3) transduced with MRB-FIV. (MRB-FIV) feline immunodeficiency virus pseudotyped with Marburg virus glycoprotein. (RLU) relative light units, (*) indicates that P<0.01.

FIG. 27 shows transduction levels of immortalized cell lines (KB, HT1080, HOS, A549, H441, HBE and IB3) with a pseudotyped FIV vector following pretreatment with an IgG1 isotype antibody (black bars) or an FRα-blocking antibody (gray bars). (EBO-FIV) feline immunodeficiency virus pseudotyped with Ebola virus glycoprotein, (EBOΔO-FIV) feline immunodeficiency virus pseudotyped with Ebola virus glycoprotein deletion mutant, (VSVG-FIV) feline immunodeficiency virus pseudotyped with vesicular stomatitis virus G protein. The mean 13-galactosidase activity following isotype antibody pretreatment for each cell line and vector administration is normalized to 100%. (*) indicates that P<0.01; (**) indicates that P<0.05.

FIG. 28 shows transduction levels of airway-derived and non-airway-derived cell lines (KB, HT1080, HOS, A549, H441, HBE and IB3) with a pseudotyped FIV vector following pretreatment with PI-PLC (gray bars) or without PI-PLC (black bars). (EBOΔO-FIV) feline immunodeficiency virus pseudotyped with Ebola virus glycoprotein deletion mutant, (VSVG-FIV) feline immunodeficiency virus pseudotyped with vesicular stomatitis virus G protein. (*) indicates that P<0.01; (**) indicates that P<0.05.

FIG. 29 illustrates the correction of CFTR defect in human cystic fibrosis (CF) epithelia following apical application of a pseudotyped virus. (EBOΔO-FIV-CFTR) feline immunodeficiency virus that expresses CFTR and is pseudotyped with an Ebola glycoprotein deletion mutant, (Ad5-CMV CFTR) adenovirus vector having a cytomegalovirus promoter operably linked to a nucleic acid sequence that encodes CFTR, (FIV-VSVG CFTR) feline immunodeficiency virus that expresses CFTR and is pseudotyped with vesicular stomatitis virus G protein, (FSK) forskolin, (IBMX) isobutylmethylxanthine, (BUMET) bumetanide.

FIG. 30A is a picture of a hematoxylin and eosin I section of well differentiated cultured human airway epithelia after 14 days in culture showing the pseudostratified epithelium and ciliated surface.

FIG. 30B are confocal microscopy images of well differentiated human airway epithelia stained with 5-chloromethylfluorescein diacetate (CMFDA).

FIG. 30C are scanning electron microscopes sections of human airway epithelia showing progressive ciliagenesis and differentiation over 14 days.

FIG. 30D shows a transmission electron microscopy (TEM) view of cultured epithelia (C) ciliated cell, (G) goblet cell, Bar=10 μm.

FIG. 30E shows a scanning electron microscopy (SEM) image of primary well differentiated cultures of mouse tracheal epithelia; Inset shows high magnification view of cilia and microvilli.

FIG. 31A illustrates the persistence of CFTR gene expression in primary cultures of human CF airway epithelia transduced on the apical surface with VSV-G-FIV-CFTR or Ad2/CFTR. (VSV-G-FIV-CFTR) feline immunodeficiency virus that carries a nucleic acid sequence that encodes CFTR and that is pseudotyped with vesicular stomatitis virus G protein, (Ad2/CFTR) adenoviral vector expressing CFTR. (ΔIsc) cAMP-activated Cl channel activity.

FIG. 31B shows the transduction of multiple epithelial cell types in vivo in rabbit trachea as assessed by β-galactosidase staining. Ciliated cells and basal cells are indicated with arrows.

FIG. 32A shows the analysis of transgene expression by HepG2 cells that were transduced with FIV-eGFP vector at MOI of 0.1. At the time-points indicated, the eGFP expression was measured by FACS analysis (left). (FIV-eGFP) feline immunodeficiency virus that expresses eukaryotic green fluorescent protein (eGFP).

FIG. 32B shows the analysis of viral integration events in HepG2 cells that were transduced with a FIV-eGFP vector at MOI of 0.1. (P) positive control; (N) negative control.

FIG. 33 illustrates the distribution of FIV integration into HepG2 RefSeq genes. The percentage of integration occurring in each portion, as well as in 5 kilobases (kb) upstream and 5 kb downstream of the gene, is shown. A dashed line indicates random integration.

FIG. 34 illustrates the analysis of the HepG2 host genomic sequences surrounding the FIV integration sites (20 basepairs (bp) upstream and 20 basepairs downstream of integration sites). Arrows indicate FIV integration sites.

FIG. 35 illustrates FIV integration sites into transduced HepG2 cells by chromosome. Each dot represents one unique integration site.

FIG. 36 illustrates FIV integration in vivo. Mice (#2, 5, 6, and 7) were injected via the tail vein with GP64/FIV. Mouse #12 received buffer injection. Liver genomic DNA was isolated at day 5 (mice #5 and 7) or day 21 (mice #2 and 6), digested with EcoRV (E) or StuI (S), and amplified with a nested PCR. Several PCR fragments were present only in the mice receiving GP64/FIV injection (indicated as green circles). These fragments were excised and sequenced. (N) negative control; (P) positive control.

FIG. 37 shows anterior and lateral pseudocolor images of luciferase expression in BALB/c mouse lung.

FIG. 38 is a diagram of the in vitro FIV transduction protocol for EBOΔO-FIV.

### DETAILED DESCRIPTION

The invention provides pseudotyped viruses and methods to use the pseudotyped viruses to deliver one or more selected nucleic acid sequences to a cell. The pseudotyped viruses of the invention include a viral capsid that is surrounded by a membrane. The viral capsid includes viral proteins and viral nucleic acid into which a selected nucleic acid sequence can be inserted. The membrane contains surface proteins, such as glycoproteins, that are displayed on the outside of the virus. These surface proteins interact with a target cell and allow the pseudotyped virus to enter into the target cell. Once within the target cell, the nucleic acid sequence contained within the capsid of the pseudotyped virus can be expressed. Accordingly, the pseudotyped viruses of the invention can be used for the delivery and expression of a selected nucleic acid sequence within a target cell.

A pseudotyped virus of the invention includes a capsid that is obtained from a different virus than the virus from which the surface protein, such as a glycoprotein, of the pseudotyped virus is obtained. The viruses of the invention are referred to as being “pseudotyped” because they include a surface protein from one or more types of viruses and the capsid of another type of virus. These pseudotyped viruses have an increased ability to enter into numerous types of target cells due to alteration of the surface protein. An increase in the range of different types of cells that a virus can infect is referred to as an expansion in the tropism of the virus.

The pseudotyped viruses of the invention can be used to deliver selected nucleic acid sequences to cells that formerly could not be infected with the virus from which the viral capsid was obtained. For example, it has been surprisingly discovered that the pseudotyped viruses of the invention are able to enter airway epithelial cells from the apical side of the airway epithelial cells and thereby introduce a selected nucleic acid sequence into the cells. An airway epithelial cell has an apical side that is exposed to air within airway structures, such as the trachea and lungs, and a basolateral side that is in contact with other cells that are buried within the airway structures and is therefore not in contact with air. The newly discovered ability to introduce a selected nucleic acid sequence into an airway epithelial cell from the apical surface of the cell greatly improves delivery of selected nucleic acid sequences to airway cells because, for example, the pseudotyped viruses can be readily administered to an animal or human through the airways by known methods. In addition, entry of a pseudotyped virus of the invention into an airway epithelial cell through the apical surface of the cell has been discovered to proceed through a mechanism that is independent of folate receptor alpha (FRα), further increasing the scope of cells that can be entered by the pseudotyped viruses of the invention.

The pseudotyped viruses and methods of the invention offer promise for the treatment of disorders having symptoms that can be reduced or eliminated through delivery of a selected nucleic acid sequence to one or more cells. An examples of such a disorder is cystic fibrosis where a pseudotyped virus of the invention can be used to deliver a copy of the cystic fibrosis transmembrane regulator protein (CFTR) to airway epithelial cells to reduce or eliminate the symptoms of cystic fibrosis.

Pseudotyped Viruses

The invention provides pseudotyped viruses. The pseudotyped viruses include a viral capsid containing viral genomic nucleic acid that is surrounded by, or enclosed within, a viral capsid. The viral capsid can include viral genomic nucleic acid that is DNA or RNA. Preferably, the viral nucleic acid included within the capsid of a pseudotyped virus of the invention is RNA. A pseudotyped virus of the invention can include a capsid that is constructed from retroviral proteins and retroviral genomic nucleic acid.

The retroviral genome can include a selected nucleic acid sequence encoding a desired protein that can be expressed within a cell that is infected with the pseudotyped retrovirus. The retroviral genome can also include other nucleic acid sequences for formation of the pseudotyped retrovirus, such as 5′ and 3′ long terminal repeat (LTR) sequences that are operably linked to the selected nucleic acid sequence encoding a desired protein. Reverse transcriptase and integrase are also enclosed within the capsid of a retrovirus, which gives the retrovirus the ability to incorporate a selected nucleic acid sequence encoding a desired protein into a genome of a cell after the retrovirus contacts, or is incubated with, the cell. For example, the pseudotyped retrovirus may be used to incorporate a selected nucleic acid sequence encoding an enzyme in a host cell that is incapable of producing the enzyme, or that produces a non-functional enzyme. Other nucleic acid sequences known to the art that are useful for transducing genes may also be present in the RNA genome.

The pseudotyped retrovirus may include other proteins, in addition to integrase, that aid its stable integration into a chromosome of a target cell. For example, with respect to a lentivirus, the pseudotyped retrovirus may include proteins such as vpr, vif and vpu.

A pseudotyped retrovirus may also include a selected nucleic acid sequence that encodes a bioactive nucleic acid, such as a bioactive RNA. Examples of bioactive RNAs include, but are not limited to, antisense nucleic acids, ribozymes and interfering RNA.

In other examples, the pseudotyped retrovirus may include a nucleotide sequence encoding a visually detectable marker. An example of a detectable marker is the Aequorea Victoria green fluorescent protein. Such a pseudotyped retrovirus may be advantageously used in a method of determining viral entry into a cell. Moreover, such a virus is advantageously used in the methods of the present invention to ensure that the pseudotyped retroviruses that are formed are replication incompetent and incapable of producing progeny retroviruses. For example, supernatant isolated from producer cells transduced by the pseudotyped viruses and contacted with a test cell should not result in localization of the fluorescent protein in the test cell.

Viral Capsid

A pseudotyped virus of the invention can include a viral capsid that is obtained from any of many different types of viruses. These viruses can be integrating viruses or non-integrating viruses. An integrating virus inserts the viral nucleic acid contained within the capsid into the genomic DNA of a target cell upon entry of the integrating virus into the target cells. A non-integrating virus will not insert the viral nucleic acid contained within the capsid of the non-integrating virus into the genomic DNA of a target cell upon entry of the non-integrating virus into the target cell.

In some examples, use of a viral capsid obtained from an integrating virus is preferred. One such example is where it is desirable to insert a selected nucleic acid sequence into the genomic DNA of a progenitor target cell such that the selected nucleic acid will be expressed in cells derived from the progenitor target cell. In another example, integration of a selected nucleic acid sequence into the genomic DNA of a target cell may be desired to obtain stable expression of the selected nucleic acid, such as in therapeutic applications.

Pseudotyped viruses of the invention can be prepared that include a capsid obtained from a retrovirus. The retroviral gag, pro and pol nucleotide sequences, and other retroviral nucleic acid sequences used for preparing the capsid in pseudotyped retroviruses may be obtained from any of a wide variety of retroviruses. Examples of such retroviruses include, but are not limited to, alpharetrovirus (i.e., avian leucosis virus), betaretrovirus (i.e., mouse mammary tumor virus), gammaretrovirus (i.e., murine leukemia virus and Moloney monkey leukemia virus), deltaretrovirus (i.e., bovine leukemia virus), epsilonretrovirus (walleye dermal sarcoma virus), lentivirus (i.e., human immunodeficiency virus I, feline immunodeficiency virus and chimpanzee foamy virus) and spumavirus.

In some examples, pseudotyped viruses are prepared using nucleic acid sequences obtained from a Moloney murine leukemia virus (MMLV of Mu-MoLV). The nucleic acid sequences of the MMLV gag, pro and pol genes are well known in the art (Bereven et al., Cell, 27:97 (1981)).

In other examples, such sequences are obtained from lentiviruses. Feline immunodeficiency virus (FIV) is an example of a lentivirus that can be used to prepare a pseudotyped virus of the invention. Unlike most retroviruses, lentiviruses have the capacity to integrate nucleic acid sequences that they carry into the chromosomes of non-dividing cells as well as dividing cells. Therefore, lentiviral nucleic acid sequences encoding proteins that allow for chromosomal integration of virally transported nucleic acid sequences into non-dividing cells are advantageously employed, as the host range of the pseudotyped retroviruses is broadened by their use.

The above-described retroviruses are readily available from the American Type Culture Collection (ATCC, Manassas, Va.) and the desired nucleic acid sequences may be obtained from these retroviruses by methods known to the skilled artisan. For example, the nucleic acid sequences may be obtained by recombinant DNA technology (Sambrook et al., Molecular Cloning: A Laboratory Manual, 3rd edition, Cold Spring Harbor Press, Cold Spring Harbor, N.Y. (2001)). Briefly, viral DNA libraries may be constructed and the nucleotide sequences may be obtained by standard nucleic acid hybridization or polymerase chain reaction (PCR) procedures using appropriate probes or primers. Alternatively, supernatant medium from cells infected with the respective virus can be isolated and the desired retroviral nucleic acid sequence may be amplified by PCR. Such vectors may also be constructed by other methods known in the art.

Viral Surface Protein (Glycoprotein (GP))

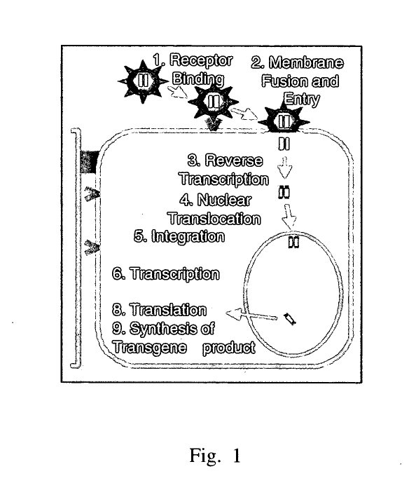

The viral surface protein included within the viral envelope assists in determining the type of cell that can be entered by a pseudotyped virus of the invention. These surface proteins are often referred to as envelope glycoproteins. The interactions between the envelope glycoprotein and a receptor on the surface of a target cell are the initial step in entry (transduction) of the target cell by a viral vector, such as a retroviral vector (FIG. 1) (Coffin et al., retroviruses. Plainview: Cold Spring Harbor Press, (2000)). Next, fusion occurs between the viral envelope and the host cell. This fusion can occur at the cell surface or in an endosomal compartment. During infection of a cell by a retrovirus, fusion of the cell and virus is followed by reverse transcription of the viral RNA into DNA and transport of the viral DNA to the nucleus where it integrates into the genome of the target cell.

Surface proteins can be obtained from numerous viruses and used to prepare the pseudotyped viruses of the invention. Examples of viruses from which glycoproteins can be derived include: togaviridea such as alphaviruses (Ross River Virus) and rubivirus; flaviviridae such as flavivirus, pestisvirus and hepatitic virus; paramyxoviridae such as morbillivirus; bunyavirus such as hantavirus; filovirus such as Ebola virus (including Ebola Zaire, Ebola Reston and Ebola Sudan sequences which are chromosomally-integrated) and Marburg virus; and oncoviridae such as Moloney Murine Leukemia virus.

Togaviral glycoproteins can be used to produce pseudotyped viruses of the invention. For example, togaviral glycoprotein pseudotyped lentiviruses are provided by the invention. The viruses include a lentiviral capsid and a viral envelope that includes a lipid bilayer and one or more functional togaviral glycoproteins. In one example, the lentivirus is a feline immunodeficiency virus (FIV) that has one or more togaviral glycoproteins imbedded in the lipid bilayer surrounding the capsid. Examples of togaviral glycoproteins include, but are not limited to, the E1 (SEQ ID NO: 6) and E2 (E3-E2 sequence indicated in SEQ ID NO: 8) envelope glycoproteins obtained from the Ross River virus (RRV) (Table 1). The E3-E2 polyprotein is synthesized and traverses the secretory system as a polyprotein precursor. The precursor is then cleaved within the Golgi apparatus to produce E2. A stable cell line producing an RRV-pseudotyped Moloney murine virus can be obtained by manipulating the E1 and E2 glycoproteins of RRV so that they are expressed by individual genes in a packaging cell system (Sharkey, C. M., et al., J. Virol. 75:2653 (2001)).

Filoviral glyproteins can be used to produce pseudotyped viruses of the invention. For example, filoviral glycoprotein-pseudotyped lentiviruses are provided by the invention. The viruses include a lentiviral capsid and a viral envelope having a lipid bilayer and a functional filoviral glycoprotein. In one example the lentivirus is a feline immunodeficiency virus (FIV). Examples of filoviruses from which a glycoprotein can be obtained include Ebola virus (EBO) and the Marburg virus (MRB).

The Ebola and Marburg viruses are a group of enveloped, single-stranded RNA viruses that are classified in the order Mononegavirales and the family Filoviridae. These viruses contain a single structural glycoprotein (GP) that forms peplomers that project from the surface of the enveloped, rod-shaped virion (Feldmann et al., Virology, 199:469 (1994); Sanchez et al., P.N.A.S. USA, 93:3602 (1996); Sanchez et al., J. Virol., 72:6442 (1998)).

The glycoproteins of filoviruses are expressed from the GP gene, but the organization of this gene differs dramatically between Ebola and Marburg viruses. The GPs of all Marburg virus isolates are encoded in a single open reading frame (ORF), whereas the GPs of Ebola viruses are encoded in two frames (0 and −1) that are connected by transcriptional editing that results in an insertion of a single base (Sanchez et al., P.N.A.S. USA, 93:3602 (1996) and Volchkov et al., Virology, 214:421(1995)). The primary gene product of the Ebola virus GP gene is a secreted GP (cleaved to generate secreted GP (SGP) and delta peptide) (Volchkova et al., Virology, 265:164 (1999)), whereas the structurally important GP is a product of the edited mRNA. Biochemical, immunological, and structural studies have clarified the role of GP in virus entry and pathogenesis (Chepurnov et al., Immunol. Lett., 68:257 (1999); Feldmann et al., Virology, 199:469 (1994); Feldmann et al., Arch. Virol. Suppl., 15:159 (1999); Ito et al., J. Virol., 73:8907 (1999); Ito et al., J. Virol., 75:1576 (2001); Malashkevich et al., P.N.A.S. USA, 96:2662-2667(1999); Ruiz-Arguello et al., J. Virol., 72:1775(1998); Sanchez et al., P.N.A.S. USA, 93:3602 (1996); Sanchez et al., J. Virol., 72:6442 (1998); Volchkov et al., P.N.A.S. USA, 95:5762 (1998); Weissenhorn et al., Mol. Cell., 2:605-616 (1998); Wool-Lewis and Bates, J. Virol., 73:1419 (1999)).

The peplomers covering the surface of Ebola virions are composed of GP trimers anchored in the lipid bilayer by a transmembrane (TM) sequence in a type I orientation (Sanchez et al., Virus Res., 29:215 (1993); Sanchez et al., P.N.A.S. USA, 93:3602 (1996); Sanchez et al., J. Virol., 72:6442 (1998)). These structures mediate the entry of the virion into cells through a process involving (i) binding to receptor molecules, (ii) endocytosis of the virion, (iii) acidification of the endocytic vesicle, and (iv) membrane fusion brought about by acid-induced conformational changes in GP (Chan et al., J. Virol., 74:4933 (2000); Ito et al., J. Virol., 73:8907 (1999); Takada et al., P.N.A.S. USA, 94:14764 (1997); Wool-Lewis et al., J. Virol., 72:3155 (1998); Yang et al., Science, 279:1034 (1998)).

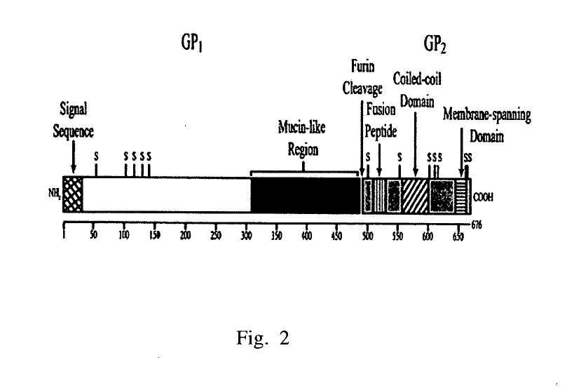

The processing of the Ebola GP in cells leads to the production of various forms of the protein as it travels through the endoplasmic reticulum (ER) and Golgi apparatus to the plasma membrane (Volchkov et al., P.N.A.S. USA, 95:5762(1998)). An N-glycosylated precursor form of the Ebola GP (GPₚᵣₑ), which is found in the ER, is further processed to a fully glycosylated uncleaved form in the Golgi apparatus (GP₀); trafficking to the Golgi apparatus also leads to the addition of O-linked glycans (Feldmann et al., Virology, 199:469 (1994); Volchkov et al., P.N.A.S. USA, 95:5762 (1998)). In the trans-Golgi apparatus, GP₀ is cleaved by the convertase furin to generate GP₁ (about 130 kDa) that is involved in receptor binding, and transmembrane GP₂ (about 24 kDa) (Sanchez et al., Virus Res., 29:215 (1993)). GP₁ and GP₂ are linked by disulfide bonding (Sanchez et al., P.N.A.S. USA, 93:3602 (1996); Sanchez et al., J. Virol., 72:6442 (1998); Volchkov et al., P.N.A.S. USA, 95:5762(1998)). FIG. 2 shows a diagrammatic view of the Zaire species of Ebola virus GP. GP₁ is highly glycosylated with N-linked and O-linked glycans (Feldmann et al., Virology, 199:469 (1994)). Glycosylation contributes approximately half of the mass of GP₁, and O-linked glycans confer a mucin-like property to its carboxyl-terminus. GP₂ also contains N-linked glycans (Sanchez et al., P.N.A.S. USA, 93:3602 (1996); Sanchez et al., J. Virol., 72:6442 (1998); Volchkov et al., P.N.A.S. USA, 95:5762(1998)) with two predicted N-linked sites but does not appear to contain O-linked glycans.

The invention provides mutant glycoproteins having a reduced level of glycosylation relative to corresponding wild-type glycoproteins. These mutant glycoproteins can contain amino acid deletions and substitutions that reduce or eliminate N-linked glycosylation, O-linked glycosylation or both N-linked glycosylation and O-linked glycosylation. Examples of such mutations are provided in Tables 3 and 5. The extent of glycosylation of a glycoprotein can be determined through use of methods known in the art and described herein (Example 3). These methods can therefore be used to determine if a mutant glycoprotein exhibits a reduced level of glycosylation when compared to another glycoprotein, such as the corresponding wild-type glycoprotein.

Disulfide bonding that holds the Ebola GP₁-GP₂ heterodimer together is predicted to involve the first cysteine of GP₁ (Cys-53) and the fifth cysteine from the amino terminus of GP₂ (Cys-609). This prediction is based on sequence and structural similarities of Ebola virus GP to the GPs of avian sarcoma and leukosis viruses (ASLVs) and other retroviruses and the fact that the Cys-53 residue of SGP is involved in forming the SGP homodimer (Gallaher, Cell, 85:477 (1996); Fass et al., Nat. Struct. Biol., 3:465 (1996); Malashkevich et al., P.N.A.S. USA, 96:2662-2667(1999); Pinter et al., J. Virol., 71:8073 (1997); Sanders (2000) Sulfhydryl involvement in fusion mechanisms, p. 483-514. In H. Hilderson and S. Fuller (ed.), Fusion of biological membranes and related problems. Kluwer Academic/Plenum Publishers, New York, N.Y.; Volchkov et al., P.N.A.S. USA, 95:5762 (1998); Sanchez et al., J. Virol., 72:6442 (1998); Volchkova et al., Virology, 250:408 (1998)). The intramolecular disulfide bonding of GP₂ has also been predicted based on sequence similarity to the avian sarcoma and leukosis virus (ASLV) GPs (Gallaher, Cell, 85:477 (1996)). A putative fusion peptide of 16 hydrophobic and uncharged residues has been identified near the amino terminus of GP₂, and a synthetic version of this peptide was shown to penetrate and induce the fusion of membranes containing phosphatidylinositol (Ruiz-Arguello et al., J. Virol., 72:1775(1998)). The membrane-spanning anchor sequence near the carboxyl-terminus of Ebola virus GP₂ contains two conserved cysteine residues that are palmitoylated (Ito et al., J. Virol., 75:1576 (2001)). X-ray crystallography of recombinant-expressed portions of GP₂ have shown that alpha helices in the sequence form coiled coils and that these structures are remarkably similar to those of the transmembrane (TM) envelope protein of retroviruses and influenza viruses as well as SNAREs (cellular proteins involved in fusion of transport vesicles) (Malashkevich et al., P.N.A.S. USA, 96:2662-2667(1999) and Weissenhorn et al., Mol. Cell., 2:605-616 (1998)).

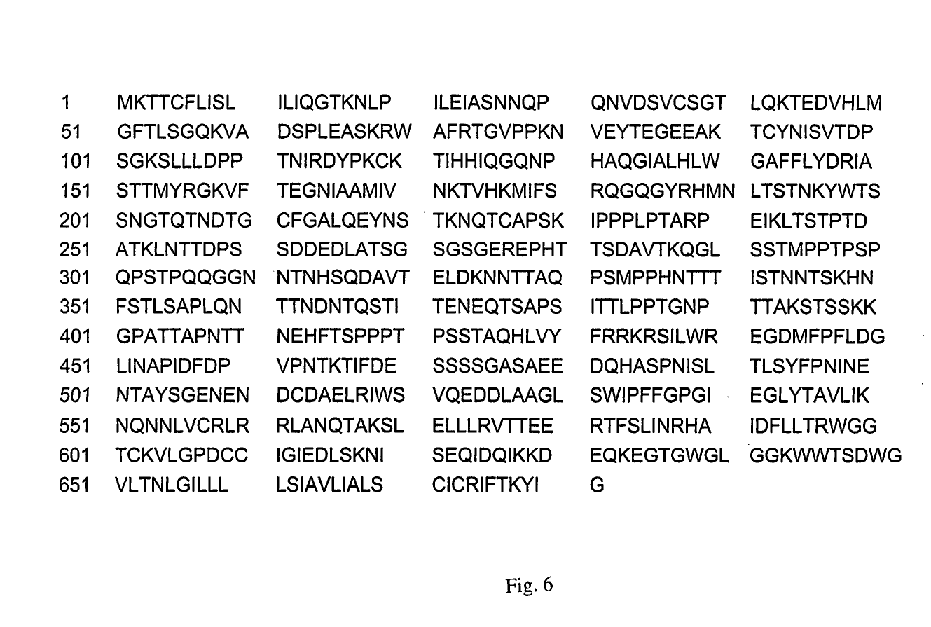

The nucleotide sequences encoding the filoviral glycoproteins may be obtained as described in Sanchez et al., Virus Res., 29:215 (1993) and Will et al., J. Virol., 67:1203 (1993). An example of an Ebola Zaire glycoprotein-encoding sequence is shown in SEQ ID NO:4 and an example of a Marburg virus glycoprotein encoding sequence is shown in SEQ ID NO:2 (FIG. 6 and Table 1). Moreover, additional sequences may be obtained by other methods known to those skilled in the art. The invention also includes nucleotide sequences that encode glycoproteins that have one or more amino acids that are different from a corresponding sequence as found in nature. Accordingly, mutant glycoproteins having insertions, deletions, and other alterations in their amino acid sequence can be used to construct the pseudotyped viruses of the invention. The nucleotide sequences that encode such glycoprotein mutants are also provided by the invention.

The invention also provides altered glycoproteins that contain mutations and deletions. These altered glycoproteins can be used to produce pseudotyped viruses of the invention. One example of an altered glycoprotein in which a portion of the amino acid sequence has been deleted is a glycoprotein in which the O-linked glycosylation (mucin-like) region has been modified or deleted in whole or in part. An example of such a glycoprotein is an Ebola virus glycoprotein in which the amino acids at positions 309 to 489 have been deleted (EBOΔO or Δ309-489).

Deletion of the O-linked glycosylation region of the Ebola GP₁ (Δ309-489) caused a surprising increase in expression and transduction of nucleic acid sequences included within viruses that displayed a GP having the O-glycosylation deletion on their surface. Prior to this surprising discovery, it was not know if a virus that included a glycoprotein in which this region was deleted would be able to enter into a target cell.

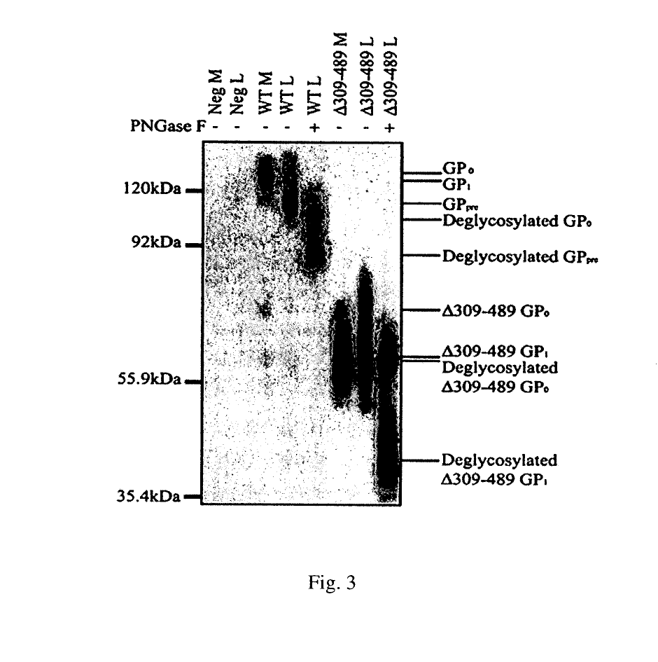

The O-linked glycosylation region, which is rich in proline, serine, and threonine residues, is the most variable among the Ebola virus GPs. Elimination of this domain resulted in enhanced GP processing and incorporation into retrovirus particles (FIG. 3). Deletion of this region also resulted in higher levels of transduction by retroviruses pseudotyped with the Ebola virus Δ309-489 GP (Table 5). Transduction by Ebola virus Δ309-489 GP-pseudotyped viruses is increased from the relatively mediocre titers achieved with wild-type GP to levels that are comparable to those achieved with standard vesicular stomatitis virus G protein-pseudotyped viruses as used with the same system. Increased virus incorporation may result from decreased processing of the protein resulting from diminution of GP toxicity (Yang et al., Nat. Med., 6:886 (2000); Simmons et al., J. Virol., 76:2518 (2002); Volchkov et al., Science, 291:1965 (2001)). The improved levels of transduction seen with the virus pseudotyped with Δ309-489 GP, combined with its potential safety advantages, provides a recombinant virus offering many advantages for gene therapy with Ebola virus GP-pseudotyped viruses, such as retroviruses or lentiviruses.

Nucleic Acid Constructs for the Production of the Pseudotyped Viruses of the Invention

The invention provides nucleic acid constructs that can be used to produce pseudotyped viruses. In one example, the invention provides a nucleic acid sequence in which the nucleic acid sequences encoding gag, pro and pol are contiguous to each other as found in native retroviral genomes, such as in the order 5′-gag-pro-pol-3′. The retroviral sequences can also be chromosomally-integrated into the cellular genome of cells used for the preparation of pseudotyped viruses of the invention. The nucleic acid sequences encoding gag, pro and pol individually or in any combination can be operably linked to a promoter so that transcription of the sequences may be obtained. Examples of the amino acid and nucleic acid sequences of feline immunodeficiency virus (FIV) and Murine Moloney Leukemia Virus (MMLV) gag and pol are known in the art (for example GenBank Accession Numbers AF033811 and NC_{—}001482).

A nucleic acid sequence is “operably linked” to another nucleic acid sequence, such as a promoter sequence, when it is placed in a specific functional relationship with the other nucleic acid sequence. The functional relationship between a promoter and an operably linked nucleic acid typically involves the nucleic acid and the promoter sequences being contiguous such that transcription of the nucleic acid sequence will be facilitated. Two nucleic acid sequences are also operably linked if the nature of the linkage between the two sequences does not (1) result in the introduction of a frame-shift-mutation; (2) interfere with the ability of the promoter region sequence to direct the transcription of the operably linked nucleic acid sequence, or (3) interfere with the ability of the desired nucleotide sequence to be transcribed from the promoter sequence region. Typically, the promoter element is upstream (i.e., at the 5′-end) of the operably linked nucleic acid sequence.

A wide variety of promoters are known in the art, including cell-specific promoters, inducible promoters, and constitutive promoters. The promoters may further be selected such that they require activation by activating elements known in the art, so that production of the protein encoded by the operably linked nucleic acid sequence may be regulated as desired. It is well within the purview of a person skilled in the art to select and use an appropriate promoter in accordance with the present invention. Examples of promoters that may be operably linked to the nucleic acid sequences that encode the gag-pro-pol proteins, include the rat actin promoter, the MMLV promoter or the cytolomegalovirus promoter. Furthermore, the cytomegalovirus promoter has been found to be an excellent promoter in the inventive system.

Other regulatory elements, such as enhancer sequences, which cooperate with the promoter and transcriptional start site to achieve transcription of an operably linked nucleic acid sequence, may also be present in the cell 5′ to the nucleotide sequences that encode retroviral proteins. By “enhancer” is meant nucleotide sequence elements, which can stimulate promoter activity in a cell, such as a bacterial or eukaryotic host cell.

Selected Nucleic Acid Sequences

A selected nucleic acid sequence can be nearly any nucleic acid sequence that can be inserted into viral nucleic acid, incorporated into a viral capsid, and packaged into a pseudotyped virus of the invention. In some examples, pseudotyped viruses of the invention having a selected nucleic acid included within the capsid can be used to transduce the selected nucleic acid sequence into a cell. Preferably, the selected nucleic acid sequence is RNA. A selected nucleic acid sequence can be a nucleic acid sequence that is, or is not, present within a cell into which the selected nucleic acid sequence is introduced. For example, a selected nucleic acid sequence can encode a protein normally present within a cell into which the selected nucleic acid is introduced, or can encode a protein that is normally not present within a cell into which the selected nucleic acid is introduced. The selected nucleic acid sequence can encode a protein or bioactive RNA that provides a beneficial or therapeutic effect when introduced into an animal. For example, a selected nucleic acid sequence may encode a protein that is not produced, that is produced in insufficient quantity, that is non-functional or that has insufficient activity within a cell. Thus, in one example, the selected nucleic acid sequence can be introduced into a cell to supplement activity that is present within the cell. In another example, a selected nucleic acid sequence can provide an activity or protein that was not present within a cell prior to entry of the pseudotyped virus. Thus, a pseudotyped virus of the invention can be used to introduce a selected nucleic acid sequence into a cell that was not present in the cell prior to entry of the cell by the pseudotyped virus. The selected nucleic acid sequence may be introduced into the viral nucleic acid of the pseudotyped virus in a variety of ways known to the skilled artisan.

In one example, the selected nucleic acid sequence encodes cystic fibrosis transmembrane regulator protein (CFTR), the chloride transporter that is involved in cystic fibrosis. The absence of CFTR function in lung epithelium due to mutations in the gene encoding CFTR, results in a severe lung disease that cannot be readily reversed or controlled by conventional treatment. Lack of CFTR function in the lung results in airway fluid with an altered ion composition, thereby creating a favorable environment for disease-causing bacteria to colonize the lung. Additionally, mucus secreted into the lung becomes thick and viscous, preventing normal clearing of the bacteria from the airways. Chronic bacterial infection of the lung leads to destruction of lung tissue and loss of lung function. Replacing the defective gene with a copy that encodes a functional CFTR can abate the symptoms.

In other examples, the selected nucleic acid sequence encodes the low-density lipoprotein (LDL) receptor, α1-antitrypsin, ornithine transcarbamylase, Factor VIII or a high affinity glutamate transporter. For example, increasing the expression of the LDL receptor in the liver allows for more efficient clearance of LDL-cholesterol from the body.

Alternatively, the selected nucleic acid sequence may encode a protein that allows entry of a virus into a cell to be detected. For example, a visually detectable marker may be one that emits visible wavelengths of light, or that can be reacted with a substrate to produce color of specified wavelengths. Examples of such selected nucleic acid sequences include the nucleic acid sequence encoding the Aequorea Victoria green fluorescent protein and the LacZ gene that encodes β-galactosidase, both of which are well known in the art and may be obtained commercially. A pseudotyped virus can include a bicistronic construct that expresses a therapeutic protein or a bioactive RNA and a marker gene (eGFP or β-galactosidase).

A selected nucleic acid can also encode a bioactive nucleic acid that is itself active, or that encodes a bioactive nucleic acid. Examples of bioactive nucleic acid include, but are not limited to, antisense nucleic acids, ribozymes, and interfering RNA.

Viral nucleic acids can be modified to improve transcription or translation of a nucleic acid sequence inserted into the viral nucleic acid. For example, the woodchuck hepatitis virus posttranscriptional regulatory element (WPRE) can increase gene expression posttranscriptionally (Zufferey et al., J. Virol., 73:2886 (1999)). Furthermore, studies with HIV-based vectors indicate that the central polypurine tract and termination sequences (cPPT) within the pol gene are required in cis for efficient nuclear translocation and the FIV cPPT has recently been identified (Barry et al., Hum. Gene Ther., 12:1103 (2001); Follenzi et al., Nat. Genet., 25:217 (2000); Whitwam et al., J. Virol., 75:9407 (2001)). Restoring cPPT sequences significantly increased viral nuclear translocation and enhanced lentiviral vector-mediated gene transfer in hematopoietic stem cells (Follenzi et al., Nat. Genet., 25:217 (2000)).

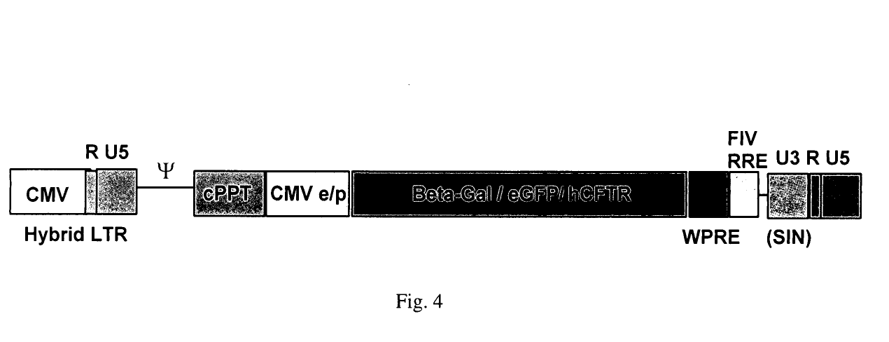

In addition, a self-inactivating (SIN) FIV transfer vector expressing eukaryotic green fluorescent protein (eGFP) was generated by deleting a portion of the U3 region of the 3′ LTR (FIG. 4) (Zufferey et al., J. Virol., 72:9873 (1998)). This deletion is transferred to the 5′ LTR after reverse transcription and integration in transduced cells, rendering the LTR in the proviruses transcriptionally inactive.

Chromosomal insulators can be incorporated into pseudotyped viruses of the invention to enhance expression, prevent silencing from surrounding chromatin structure, and to insulate neighboring genomic DNA from the influence of integrated viral nucleic acid. Chromatin insulators are naturally occurring elements in the genome that function as boundary elements to separate distinct chromatin domains (Bell et al., Science, 291:447 (2001) and Emery et al., P.N.A.S. USA, 97:9150 (2000)). Insulators provide a barrier against incursions from surrounding domains, and thereby protect against position effects and/or to block distal enhancer activity. Moreover, insulators may protect the surrounding chromatins against the interference from integrated viral nucleic acid sequences, thereby reducing the risk of activation of nearby oncogenes. A 1.2-kb β-globin cHS4 insulator was obtained from Dr. Gary Felsenfeld at NIH and inserted into the 3′ LTR of a recombinant FIV based vector.

Scaffold matrix attachment regions (SMARs) can be incorporated into recombinant viruses and pseudotyped viruses of the invention. SMARs are cis-acting elements that function outside transcribed regions and in introns. These elements partition the eukaryotic genome into functional loop domains. SMARs bind to the ubiquitous components of the nuclear matrix such as lamins and matrins, accommodate specific transcription factors, and recruit the replication apparatus to the origin of replication (Agarwal et al., J. Virol., 72:3720 (1998) and Schubeler et al., Biochemistry, 35:11160 (1996)). Moreover, SMARs may also define boundaries of chromatin domains, allowing for position-independent, autonomous transgene expression. Incorporation of the human interferon-β SMAR elements (IFN-SMARs) into a FIV-based virus' of the invention increased transgene expression by 2 orders of magnitude. The plasmid containing the 800 base pair IFN-SMAR was obtained from Dr. Jurgen Bode at the German Research Center for Biotechnology and inserted into a recombinant FIV vector.

Cells for the Production of Pseudotyped Viruses of the Invention

Any cell that can be transformed with nucleic acids that encode viral capsid proteins and produce viral particles or viruses can be used to produce the pseudotyped viruses of the invention. Examples of cells that can be used as producer cells include eukaryotic cells, such as mammalian cells. Examples of mammalian cells include human, simian, canine, feline, equine and rodent cells. Human cells are most preferred. It is further preferred that the cell be able to reproduce indefinitely, and is therefore immortal. Specific examples of cells include NIH3T3 cells, COS cells, Madin-Darby canine kidney cells and human embryonic 293T cells. Highly transfectable cells, such as human embryonic kidney 293T cells, are preferred. By “highly transfectable” it is meant that at least about 50%, more preferably at least about 70% and most preferably at least about 80% of the cells can express the genes of the introduced DNA. The term “eukaryotic cell line” as used herein is intended to refer to eukaryotic cells that are grown in vitro.

In one example of the present invention, eukaryotic cells may include a nucleic acid sequence that encodes a desired protein so that they can produce pseudotyped retroviruses having an RNA genome that includes the desired nucleic acid sequences. The protein can be such that it provides a beneficial or therapeutic effect if introduced into an animal or human. For example, a nucleic acid sequence may encode a protein that is needed by an animal or human, either because the protein is no longer produced, is produced in insufficient quantities to be effective in performing its function, or is mutated such that it either no longer functions or is only partially active for its intended function. The nucleotide sequence may be introduced into the cellular genome in a variety of ways known to the skilled artisan. For example, defective retroviruses that do not have the capability to produce all of the viral proteins necessary for production of a retrovirus having the ability to infect a cell and produce progeny viruses, may be constructed to include such a nucleic acid sequence in their RNA genome. These defective retroviruses can be used to transduce the nucleic acid sequence into a target cell. Alternatively, plasmid vectors may be used to introduce the nucleic acid sequence, preferably DNA, encoding the desired protein. In either case, the vector typically includes nucleotide sequences necessary for production of the pseudotyped retrovirus. For example, the RNA sequence in the viral genome is flanked on the 5′ end by a splice acceptor site and a splice donor site followed by a sequence necessary for packing of the viral genome, such as a psi sequence, and a long terminal repeat (LTR), all as known in the art. The 3′ end of the RNA sequence may be flanked on its 3′ end with a polypurine tract followed by another LTR, as known to the skilled artisan. The vectors may include other nucleic acid sequences known to the art that are necessary for transduction.

Methods to Produce Cells that can be Used to Produce Pseudotyped Viruses

The invention provides methods to create cells for the production of pseudotyped viruses. The methods involve introducing nucleic acid sequences that encode viral capsid proteins and viral glycoproteins into a cell such that the cell is able to produce a pseudotyped virus. Nucleic acid sequences encoding the viral capsid proteins may be obtained from a retrovirus, such as a lentivirus. Nucleic acid sequences that encode viral glycoproteins can be obtained from numerous viruses, such as togaviruses and filoviruses.

In one example, the method includes introducing nucleic acid sequences into the cells, such as those encoding retroviral Gag, Pro and Pol polypeptides; and nucleic acid sequences that encode a filoviral glycoprotein, such as one in which the O-glycosylation domain is deleted or mutated, in part or whole, into the cell.

The nucleic acid sequences may be introduced into the desired cell utilizing a variety of vectors known to the skilled artisan. For example, plasmid vectors, cosmid vectors, and other viral vectors, such as retroviral vectors, may be used. It is preferred that the nucleic acid sequences encoding the Gag, Pro and Pol polypeptides are on a separate vector than the nucleic acid sequences encoding the viral glycoproteins. In one mode of practicing the invention, plasmid vectors are advantageously used to introduce, or transfect, the nucleic acid sequences into the selected cell. A wide variety of plasmid vectors may be used, including pTRE, pCMV-Script and pcDNA3, although pcDNA3 is a preferred vector. The gag, pro and pol nucleic acid sequences are preferably on the same plasmid, and, as discussed herein, are preferably contiguous to each other. However, other spatial configurations of the nucleic acid sequences may be utilized when constructing the plasmids. The vector can also include a promoter upstream from the gag nucleic acid sequence. The vectors may further include other regulatory elements, such as enhancer sequences, as discussed herein.

The viral glycoprotein encoding sequences, such as those encoding filoviral or togaviral glycoproteins, are preferably operably linked to a promoter. Nucleic acid sequences encoding the one or more different viral glycoproteins may be arranged on a vector such that the nucleic acid sequence encoding one of the glycoproteins is present on one vector and the nucleic acid sequence encoding the other glycoprotein is present on a different vector. Preferably the nucleic acid sequences are on the same vector, and preferably contiguous to each other so they will be transcribed utilizing the same promoter. The cytomegalovirus promoter is one example of a promoter that can be operably linked to a nucleic acid sequence that encodes a glycoprotein or a viral capsid protein. Plasmids, or other vectors carrying the nucleic acid sequences encoding the viral glycoproteins, may also include other regulatory elements, such as enhancers, as described herein.

Vectors may be introduced into cells in a variety of ways known to the skilled artisan, for example, those discussed in Current Protocols in Molecular Biology, John Wiley and Sons, edited by Ausubel et al. (1988) and Maniatis, et al., Molecular Cloning A Laboratory Manual, Cold Spring Harbor Laboratory (1989). For example, vectors may be transfected into a cell by a calcium phosphate precipitation method. Other methods for introduction of the vectors include, for example, electroporation and lipofection.

Cells that have been transfected with a vector can be quickly visualized through use of a detectable marker such as the Aequorea Victoria green fluorescent protein. When creating a cell that includes a detectable marker, the nucleotide sequences encoding the marker may also be introduced into the cell as described above. Cells that have taken up the vector and express the nucleotide sequences encoding a protein may be identified and, separated from cells that do not express the sequences by a fluorescence activated cell sorting procedure as known in the art. A visually detectable marker may also be formed upon reaction of the product of a marker gene with a substrate. For example, cells that express β-galactosidase can be detected by contacting the cells with an X-gal substrate.

Delivery of a Selected Nucleic Acid Sequence to a Target Cell

The present invention provides methods for introducing a selected nucleic acid sequence into a target cell using a pseudotyped virus. Generally, the method involves contacting a target cell with a pseudotyped virus that contains a nucleic acid sequence into which the selected nucleic acid sequence has been inserted. The pseudotyped virus then enters the target cell whereupon the selected nucleic acid is expressed. The selected nucleic acid can encode a therapeutic protein or a bioactive nucleic acid as known in the art or described herein. The selected nucleic acid can integrate into the genomic DNA of the target cell, be maintained episomally or be expressed transiently. Preferably the selected nucleic acid integrates into the genomic DNA of the target cell with the viral nucleic acid. Preferably, a filoviral or togaviral glycoprotein pseudotyped lentiviruses is used to deliver selected nucleic acid sequences to a target cell.

Nearly any cell that can be entered by a pseudotyped virus can be used as a target cell. Examples of such cells include hepatocytes, brain glial cells or airway epithelial cells. The cells may be contacted with the pseudotyped virus in vivo, in vitro or ex vivo. Cells can be contacted with a pseudotyped virus in vitro and then implanted in an animal or human. Cells can also be harvested from an animal or human, a selected nucleic acid sequence can then be transduced into the harvested cells using a pseudotyped virus, and the transduced cells can then be implanted into the animal or human from which they were harvested. Such a method is thought to minimize immunological rejection of the transduced cells upon implantation of the cells in the animal or human.

The pseudotyped viruses can be administered to a mammal requiring gene therapy through use of a number of methods known to the skilled artisan. For example, to transduce a nucleic acid sequence into airway epithelium, the viruses can be introduced directly into the airway by inhalation aided by a nebulizer or an inhaler. The pseudotyped viruses of the present invention can also be injected intravenously for systemic gene delivery. The pseudotyped viruses can also be injected directly into the liver or the brain parenchyma. Pseudotyped viruses of the invention can also be formulated as compositions, such as pharmaceutical compositions, to facilitate delivery of the pseudotyped virus to a target cell.

In one example, pseudotyped viruses that contain a selected nucleic acid sequence that encodes the cystic fibrosis transmembrane conductance regulator (CFTR) protein are contacted with the apical surface of airway epithelial cells in an animal or human having cystic fibrosis to reduce or eliminate the symptoms of cystic fibrosis. It has been surprisingly discovered that the pseudotyped viruses of the invention are able to enter airway epithelial cells from the apical side of the airway epithelial cells and thereby introduce a selected nucleic acid sequence into the cells. An airway epithelial cell has an apical side that is exposed to air within airway structures, such as the trachea and lungs, and a basolateral side that is in contact with other cells that are buried within the airway structures and is therefore not in contact with air. The ability to introduce a selected nucleic acid sequence into an airway epithelial cell from the apical surface of the cell provides for improved delevery of one or more selected nucleic acid sequences to the airway cell because, for example, it allows the pseudotyped virus to be administered to an animal or human through an airway mediated route. Examples of such routes include the use of inhalers, nebulizers, sprays and the like. Lentiviruses pseudotyped with filoviral glycoproteins in which a portion of the O-glycosylation region has been deleted are thought to be useful for introducing nucleic acid sequences into airway epithelial cells.

In another example, the method includes contacting, or transducing, a hepatocyte or brain glial cell with a lentivirus that has been pseudotyped with togaviral glycoproteins that includes the desired nucleic acid sequence in its genome.

In another example, the cells are brain glial cells. One type of glial cell, oligodendrocytes, is responsible for formation of the myelin sheath that protects the spinal cord. In multiple sclerosis, both oligodendrocytes and the myelin sheath are destroyed. Another type of glial cell, an astrocyte, contains high affinity glutamate transporters that are critical in maintaining the extracellular glutamate concentration at sub-excitotoxic levels and thereby prevent neuronal cell death. Insufficient glutamate uptake by the transporters is believed to play a role in numerous diseases that include, amyotrophic lateral sclerosis, Alzheimer's disease, schizophrenia, and AIDS. Astrocytic uptake of glutamate may also serve to fine-tune the time course of glutamate in the synaptic cleft, perhaps by terminating the synaptic signal. Additionally, astrocytes may mediate inter-synaptic spillover of glutamate. The togaviral glycoprotein pseudotyped lentiviruses of the present invention are selective for transducing glial cells as compared to other CNS cells. Thus, the pseudotyped viruses of the invention may be administered to brain cells to transduce a nucleic acid sequence that expresses a high affinity glutamate transporter.

The pseudotyped viruses of the invention can be used to transduce cells with nucleic acid sequences that encode numerous therapeutic proteins that are known in the art and exemplified by, the LDL receptor, α1-antitrypsin, ornithine transcarbamylase, Factor VIII or a high affinity glutamate transporter.

Use of Pseudotyped Viruses for Treatment of Disorders

The pseudotyped viruses of the invention can be used to reduce or eliminate the symptoms of numerous disorders in animals and humans by delivering a nucleic acid sequence that encodes a therapeutic protein or bioactive nucleic acid to the animal or human in need of such treatment. Examples of disorders that can be corrected through delivery of a therapeutic nucleic acid include, but are not limited to, α1-antitrypsin deficiency, hypercholesterolemia, phenylketonuria, cystic fibrosis and urea cycle disorders (Grasso and Wu, Adv. Pharm., 28:169 (1994)).

In one example, a pseutotyped virus of the invention, into which a nucleic acid sequence that encodes CFTR, can be administered to the pulmonary epithelium of a patient having cystic fibrosis. Transfer of the CFTR gene to the pulmonary epithelium with an integrating vector may correct the CFTR defect in a population of progenitor cells that could pass the corrected gene to their progeny. There appear to be several epithelial cell types in the lung that provide these functions. These cells may represent a pluripotent or “stem cell” population or serve as progenitors for a specific lineage of cells. Experiments from several species and model systems have identified such cells: basal cells and non-ciliated columnar cells of the airways, submucosal glands, alveolar type II cells and Clara cells in the distal lung (Ford and Terzaghi-Howe, Exp. Cell Res., 198:69 (1992); Randell, Chest, 101:11S (1992); Borthwick et al., Am. J. Respir. Cell Mol. Biol., 24:662 (2001); Engelhardt et al., Development, 121:2031 (1995); Evans et al., Lab. Invest., 35:246 (1976); Hong et al., Am. J. Respir. Cell Mol. Biol., 24:671 (2001), Adamson et al., Lab. Invest., 30:35 (1974); Evans et al., Exp. Mol. Path., 22:142 (1975)). Regional stem cell niches have been defined in the airways of the lung that include non-ciliated Clara cell secretory protein positive (CCSP-positive) surface cells near submucosal gland duct openings and between cartilage rings in the proximal airways, and CCSP-positive Clara cells of the distal airways (Borthwick et al., Am. J. Respir. Cell Mol. Biol., 24:662 (2001); Engelhardt, Am. J. Respir. Cell Mol. Biol., 24:649 (2001); Hong et al., Am. J. Respir. Cell Mol. Biol., 24:671 (2001)). Ciliated cells are generally thought to be terminally differentiated and not to represent a progenitor population. Since some ciliated cells in the human airways are CCSP-positive, it has been suggested that some of these cells may have a progenitor role (Engelhardt, Am. J. Respir. Cell Mol. Biol., 24:649 (2001); Englehardt et al., J. Clin. Invest., 93:737 (1994); Erjefalt et al., Cell Tissue Res., 281:305 (1995); Lawson et al., Am. J. Pathol., 160:315 (2002)).

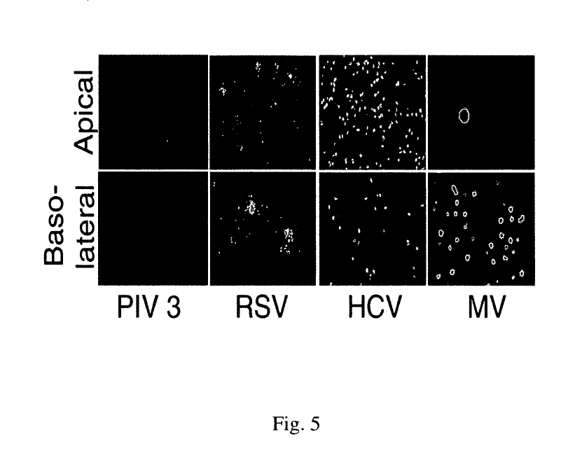

Many enveloped RNA viruses recognized as respiratory tract pathogens use the lung as their initial portal of entry and presumably enter via the apical surface. Examples include members of the Paramyxoviridae family of viruses (e.g., parainfluenza (PIV), respiratory syncytial virus (RSV), measles virus), and the Orthomyxovirus (influenza) and Coronavirus families. For example, wild type human coronavirus 229E and influenza virus both readily enter and infect airway epithelia when applied apically (Slepushkin et al., Mol. Ther., 3:395 (2001) and Wang et al., J. Virol., 74:9234 (2000)). Wild type virus or a replication competent reporter virus (measles (Schneider et al., J. Virol., 74:9928 (2000)) was applied to the apical or basolateral surface of well differentiated human airway epithelia and 24 hours later immunostained for viral proteins or visualized transgene expression (FIG. 5). While PIV3, RSV and coronavirus could infect apically, the Edmonston vaccine strain of measles virus infected airway epithelia much better from the basolateral surface. These results indicate that there is no absolute barrier to the entry of enveloped viruses on human airway epithelia. Therefore, a retroviral vector could enter via the apical surface, if an appropriate receptor is targeted.

Initial steps in retroviral transduction require interactions between the viral envelope glycoprotein (GP) and receptors on the host cell. However, for many viral vectors, the cellular receptors are functionally absent from the apical surface of airway epithelia. The absence of viral receptors disallows viral entry into airway cells from the apical side. However, disruption of the epithelial tight junctions with EGTA allows apically applied vectors to access receptors on the basolateral side. A pseudotyped FIV vector was created using glycoproteins from the filoviruses (Marburg/Ebola), and modifying the glycoproteins to improve vector titer (Sinn et al., J. Virol., 77:5902 (2003)). The FIV vector pseudotyped with a filovirus envelope preferentially entered polarized human airway epithelia from the apical surface without the disruption of tight junctions. Accordingly, the invention provides pseudotyped viruses that can be used to deliver nucleic acid sequences, such as the CFTR gene, to airway epithelial cells when applied to the apical side of such cells.

Compositions

The invention provides compositions that can be used for the administration of one or more pseudotyped viruses of the invention to an animal or human in need thereof. In one example, a composition can contain one or more pseudotyped viruses of the invention, and optionally a pharmaceutically acceptable carrier.

The compositions of the invention may be prepared in many forms that include tablets, hard or soft gelatin capsules, aqueous solutions, suspensions, and liposomes and other slow-release formulations, such as shaped polymeric gels. An oral dosage form may be formulated such that a pseudotyped virus is released into the intestine after passing through the stomach. Such formulations are described in Hong et al., U.S. Pat. No. 6,306,434 and in the references contained therein.

Oral liquid compositions may be in the form of, for example, aqueous or oily suspensions, solutions, emulsions, syrups or elixirs. Such liquid compositions may contain conventional additives such as suspending agents, emulsifying agents, non-aqueous vehicles (which may include edible oils), or preservatives.

A pseudotyped virus can be formulated for parenteral administration (e.g., by injection, for example, bolus injection or continuous infusion) and may be presented in unit dosage form in ampules, prefilled syringes, small volume infusion containers or multi-dose containers with an added preservative. The compositions may take such forms as suspensions, solutions, or emulsions in oily or aqueous vehicles, and may contain formulatory agents such as suspending, stabilizing and/or dispersing agents.

Compositions suitable for rectal administration can be prepared as unit dose suppositories. Suitable carriers that may be included in the composition include those exemplified by saline solutions and other materials commonly used in the art.

For administration by inhalation, a pseudotyped virus can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for intra-nasal administration, a pseudotyped virus may be administered via a liquid spray, such as via a plastic bottle atomizer.

A pseudotyped virus can also be formulated as an aqueous solution, suspension or dispersion, an aqueous gel, a water-in-oil emulsion, or an oil-in-water emulsion.

Compositions of the invention may also contain other ingredients such as flavorings, colorings, anti-microbial agents, and preservatives. Some compositions of the invention will include an agent that disrupts junctions between cells. Examples of such agents include calcium chelators such as EDTA, EGTA and sodium citrate. In addition, a composition of the invention can include pharmaceutically active ingredients.

Examples of pharmaceutically active ingredients include Ace inhibitors, adrenergic agonists, adrenergic blockers, adrenocortical steroids, adrenocortical suppressants, adrenocorticotropic hormones, alcohol deterrents, aldose reductase inhibitors, aldosterone antagonists, reductase inhibitors, AMPA receptor antagonists, anabolic steroids, analeptics, analgesics, androgens, anesthetics, antihypertensives, anorexics, antacids, anthelmintics, antiacne compounds, antiallergics, antialopecia agents, antiameobic, antiandrogens, antianginals, antiarrhythmics, antiarteriosclerotics, antiarthritics, antirheumatics, antiasthmatics, antibiotics, antineoplastics, anticholelithogenics, anticholesteremics, anticoagulants, anticonvulsants, antidepressants, antidiabetics, antidiarrheals, antidiuretics, antidotes, antidyskinetics, antieczematics, antiemetics, antiepileptics, antiestrogens, antifibrotics, antiflatulants, antifungals, antiglaucomas, antigonadotropins, antigouts, antihemophilic factors, antihemorrhagics, antihistamines, antihypercholesterolemics, antihyperlipidemics, antihyperlipoproteinimics, antihyperparathyroids, antihyperphosphatemics, antihypertensives, antihyperthyroids, antihypotensives, antihypothyroids, anti-infectives, anti-inflammatories, antileprotic, antileukemics, antilipemics, antilipidemics, antimalarials, antimanics, antimethemoglabbinemics, antimigraines, antimuscarinics, antimycotics, antinauseants, antineoplastics, antineutropenics, antiobesity agents, antiobsessionals, antiosteoporotics, antipagetics, antiparkinsonians, antiperistaltics, antipheochromocytomas, antipneumocystics, antiprogestins, antiprostatic hypertrophies, antiprotozoals, antipruritics, antipsoriatics, antipsychotics, antipyretics, antirheumatics, antirickettsials, antiseborrheics, antisepsis agents, antiseptics, disinfectants, antispasmotics, antisyphilitics, antithrombocythemics, antithrombotics, antituberculars, antitumor agents, antitussives, antiulceratives, antiurolothics, antivenins, antivertigo agents, antivirals, anxiolytics, aromatase inhibitors, astringents, antriopeptidase inhibitors, benzodiazepine antagonists, beta-blockers, bone resorption inhibitors, bradycardiac agents, bradykinin antagonists, bronchodilators, calcium channel blockers, calcium regulators, calcium supplements, carbonic anhydrase inhibitors, cardiac depressants, cardioprotectives, cardiotonics, cathartics, CCK antagonists, chelating agents, cholelitholytic agents, choleretics, cholinergics, cholinesterase inhibitors, cholinesterase reactivators, CNS stimulants, cognitive activators, COMT inhibitors, contraceptives, cyclooxygenase-2 inhibitors, cytoprotectants, debriding agents, decongestants, dental plaque inhibitors, depigmentors, dermatitis herpetiforms suppressants, digestive aids, diuretics, dopamine receptor agonists, dopamine receptor antagonists, ectoparasiticides, emetics, endotherin receptor antagonists, enkephalinase inhibitors, enzymes, enzyme cofactors, enzyme inducers, estrogens, estrogen antagonists, expectorants, fibrinogen receptor antagonists, gastric and pancreatic secretion stimulants, gastric proton pump inhibitors, gastric secretion inhibitors, gastroprokenetics, glucocorticoids, alpha-glucosidase inhibitors, gonad-stimulating agents, growth hormone antagonists, growth hormone inhibitors, growth hormone releasing factors, growth stimulation agents, hematinics, hematopoietics, hemolytics, hemorheologic agents, hemostatic agents, heparin antagonists, hepatoprotectants, histamine H1-receptor antagonists, histamine H2-receptor antagonists, anti-HIV agents, HMG COA reductase inhibitors, hypnotic agents, immunomodulators, immunosuppressants, insulin sensitizers, keratolytics, lactation stimulating hormones, laxatives, leukotriene antagonists, LH-RH agonists, LH-RH antagonists, lipotropics, 5-lipoxygenase inhibitors, lupus erythematosus suppressants, tranquilizers, metalloprotease inhibitors, mineralocorticoids, miotics, monoamine oxidase inhibitors, mucolytics, muscle relaxants, mydriatics, narcotics, narcotic antagonists, nasal decongestants, neuraminidase inhibitors, neuroleptics, neuromuscular blocking agents, neutral endopeptidase inhibitors, neuroprotectives, NMDA receptor antagonists, nootropics, ovarian hormones, oxytocics, pepsin inhibitors, phosphodiesterase inhibitors, pigmentation agents, plasma volume expanders, platelet activation factor agonists, potassium channel activators, potassium channel blockers, pressor agents, progesterones, prolactin inhibitors, prostaglandins, prostaglandin analogs, protease inhibitors, pulmonary surfactants, respiratory stimulants, reverse transcriptase inhibitors, sclerosing agents, sedatives, serenics, serotonin noradrenaline reuptake inhibitors, serotonin receptor agonists, serotonin receptor antagonists, serotonin reuptake inhibitors, sialagogues, somatostatin analogs, succinylcholine synergists, thrombolytics, thromboxane A2-receptor antagonists, thromboxane A2-synthetase inhibitors, thyroid hormones, thyroid inhibitors, thyrotropic hormones, tocolytics, topical protectants, topoisomerase I inhibitors, topoisomerase II inhibitors, tranquilizers, uricosurics, vasodilators, vasopeptidase inhibitors, vasoprotectants, vulnerarys, xanthine oxidase inhibitors, and the like. Numerous therapeutic agents are known in the art (Merck Index, Merck Research Laboratories, 13^{th} edition, Whitehouse Station, NJ (2001); Physicians Desk Reference, Thompson P D R, 58^{th} edition, Des Moines, Iowa (2004); Mosbys 2004 Drug Guide, Mosby Inc., St. Louis, Mo. (2004)).

It will be appreciated that the amount of a pseudotyped virus required for use in treatment will vary not only with the particular carrier selected but also with the route of administration, the nature of the condition being treated and the age and condition of the patient. Ultimately the attendant health care provider may determine proper dosage. In addition, a composition of the invention may be formulated as a single unit dosage form.

Screening Agents that Block Viral Entry into a Cell

The present invention also provides methods of screening candidate agents effective in blocking viral entry into a cell. The methods allow for direct screening as the viral entry step can be detected in the method. In one example, the method includes treating the cell or the pseudotyped virus with a candidate agent, contacting the cell with the pseudotyped virus, and detecting entry of the pseudotyped virus into the cell. A wide variety of candidate agents may advantageously be screened in the present invention, including, immunological agents such as monoclonal and/or polyclonal antibodies. Alternately, various pharmacological agents may also be screened in the present method in the same way, and may include proteins, peptides and various chemical agents. Pseudotyped viruses of the invention that express a detectable marker are useful in the method. In addition, pseudotyped viruses of the invention in which the glycoprotein lacks the O-glycosylation region are also thought to be useful.

Kits

The invention provides kits for forming the pseudotyped viruses of the invention. The kits contain packaging material and plasmids and nucleic acid sequences required to transform a cell to produce a desired virus.

TABLE 1

Examples of amino acid and nucleic acid
sequences for glycoproteins and capsid
proteins. Nucleotide Sequences are shown
from the 5′ to the 3′ direction.

SEQ
ID

| | | |
|---|---|---|
| NO: | Description | SEQ ID NO: |

| | | |
|---|---|---|
| 1 | Amino acid | MKTTCFLISLILIQGTKNLPILEIASNNQPQNVDSVCSG |
| | sequence for | TLQKTEDVHLMGFTLSGQKVADSPLEASKRWAFRTG |
| | Marburg virus | VPPKNVEYTEGEEAKTCYNISVTDPSGKSLLLDPPTNI |
| | glycoprotein | RDYPKCKTIHHIQGQNPHAQGIALHLWGAFFLYDRIAS |
| | (GenBank | TTMYRGKVFTEGNIAAMIVNKTVHKMIFSRQGQGYR |
| | Accession Number | HMNLTSTNKYWTSSNGTQTNDTGCFGALQEYNSTKN |
| | Z12132) | QTCAPSKIPPPLPTARPEIKLTSTPTDATKLNTTDPSSD |
| | | DEDLATSGSGSGEREPHTTSDAVTKQGLSSTMPPTPSP |
| | | QPSTPQQGGNNTNHSQDAVTELDKNNTTAQPSMPPH |
| | | NTTTISTNNTSKHNFSTLSAPLQNTTNDNTQSTITENEQ |
| | | TSAPSITTLPPTGNPTTAKSTSSKKGPATTAPNTTNEHF |
| | | TSPPPTPSSTAQHLVYFRRKRSILWREGDMFPFLDGLI |
| | | NAPIDFDPVPNTKTIFDESSSSGASAEEDQHASPNISLT |
| | | LSYFPNINENTAYSGENENDCDAELRIWSVQEDDLAA |
| | | GLSWIPFFGPGIEGLYTAVLIKNQNNLVCRLRRLANQT |
| | | AKSLELLLRVTTEERTFSLINRHAIDFLLTRWGGTCKV |
| | | LGPDCCIGIEDLSKNISEQIDQIKKDEQKEGTGWGLGG |
| | | KWWTSDWGVLTNLGILLLLSIAVLIALSCICRIFTKYIG |
| 2 | Nucleic acid | ATGAAGACCACATGTCTCTTTATCAGTCTTATCTTA |
| | sequence for | ATCCAAGGGATAAAAACTCTCCCTATTTTAGAGATA |
| | Marburg virus | GCTAGTAACAATCAACCCCAAAATGTGGATTCGGTA |
| | glycoprotein | TGCTCCGGAACTCTCCAGAAGACAGAAGATGTCCA |
| | (GenBank | TCTGATGGGATTCACACTGAGTGGGCAAAAAGTTGC |
| | Accession Number | TGATTCCCCTTTGGAGGCATCCAAGCGATGGGCTTT |
| | NC_001608) | CAGGACAGGTGTACCTCCCAAGAATGTTGAGTATAC |
| | | AGAAGGGGAGGAAGCCAAAACATGCTACAATATAA |
| | | GTGTAACGGATCCCTCTGGAAAATCCTTGCTGTTGG |
| | | ATCCTCCTACCAACATCCGTGACTATCCTAAATGCA |
| | | AAACTATCCATCATATTCAAGGTCAAAACCCTCATG |
| | | CGCAAGGGATCGCCCTCCATTTGTGGGGAGCATTTT |
| | | TCCTGTATGATCGCATTGCCTCCACAACAATGTACC |
| | | GAGGCAGAGTCTTCACTGAAGGGAACATAGCAGCT |
| | | ATGATTGTCAATAAGACAGTGCACAAAATGATTTTC |
| | | TCGAGGCAAGGACAGGGGTACCGTCACATGAATCT |
| | | GACTTCTACTAATAAATATTGGACAAGTAACAATGG |
| | | AACACAAACGAATGACACTGGATGCTTCGGTGCTCT |
| | | TCAAGAATACAACTCCACGAAGAATCAAACATGTG |
| | | CTCCGTCCAAAATACCCTCACCACTGCCCACAGCCC |
| | | GTCCAGAGATCAAACCCACAAGCACCCCAACTGAT |
| | | GCCACCACACTCAACACCACAGACCCAAACAATGA |
| | | TGATGAGGACCTCATAACATCCGGTTCAGGGTCCGG |
| | | AGAACAGGAACCCTATACAACTTCAGATGCGGTCA |
| | | CTAAGCAAGGGCTTTCATCAACAATGCCACCCACTC |
| | | CCTCACCACAACCAAGCACGCCACAGCAAGAAGGA |
| | | AACAACACAGACCATTCCCAAGGTACTGTGACTGA |
| | | ACCCAACAAAACCAACACAACGGCACAACCGTCCA |
| | | TGCCCCCCCACAACACCACTGCAATCTCTACTAACA |
| | | ACACCTCCAAGAACAACTTCAGCACCCTCTCTGTAT |
| | | CACTACAAAACACCACCAATTACGACACACAGAGC |
| | | ACAGCCACTGAAAATGAACAAACCAGTGCCCCCTC |
| | | GAAAACAACCCTGCCTCCAACAGGAAATCTTACCA |
| | | CAGCAAAGAGCACTAACAACACGAAAGGCCCCACC |
| | | ACAACGGCACCAAATATGACAAATGGGCATTTAAC |
| | | CAGTCCCTCCCCCACCCCCAACCCGACCACACAACA |
| | | TCTTGTATATTTCAGAAAGAAACGAAGTATCCTCTG |
| | | GAGGGAAGGCGACATGTTTCCTTTTCTGGACGGGTT |
| | | AATAAATGCTCCAATTGATTTTGATCCAGTTCCAAA |
| | | TACAAAGACGATCTTTGATGAATCTTCTAGTTCTGG |
| | | TGCTTCGGCTGAGGAAGATCAACATGCCTCCCCCAA |
| | | TATCAGTTTAACTTTATCCTATTTTCCTAATATAAAT |
| | | GAAAACACTGCCTACTCTGGAGAAAATGAGAACGA |
| | | TTGTGATGCAGAGTTAAGAATTTGGAGCGTTCAGGA |
| | | GGATGACCTGGCAGCAGGGCTCAGTTGGATACCGTT |
| | | TTTTGGCCCTGGAATCGAAGGACTTTATACTGCTGG |
| | | TTTAATTAAAAACCAAAACAATTTGGTCTGCAGGTT |
| | | GAGGCGTCTAGCCAATCAAACTGCCAAATCCTTGG |
| | | AACTCTTATTAAGAGTCACAACCGAGGAAAGGACA |
| | | TTTTCCTTAATTAATAGACATGCCATTGACTTTCTAC |
| | | TCACAAGGTGGGGAGGAACATGCAAAGTGCTTGGA |
| | | CCTGATTGTTGCATTGGAATAGAAGACTTGTCCAGG |
| | | AATATTTCGGAACAAATTGACCAAATCAAAAAAGA |
| | | TGAACAAAAAGAGGGGACTGGTTGGGGTCTAGGTG |
| | | GTAAATGGTGGACATCCGACTGGGGTGTTCTTACTA |
| | | ACTTGGGCATTTTGCTACTATTATCCATAGCTGTCTT |
| | | GATTGCTCTATCCTGTATTTGTCGTATCTTTACCAAA |
| | | TATATCGGGTAA |
| 3 | AA sequence for | MGVTGILQLPRDRFKRTSFFLWVIILFQRTFSIPLGVIH |
| | Ebola virus | NSTLQVSDVDKLVCRDKLSSTNQLRSVGLNLEGNGV |
| | glycoprotein | ATDVPSATKRWGFRSGVPPKVVNYEAGEWAENCYNL |
| | (GenBank | EIKKPDGSECLPAAPDGIRGFPRCRYVHKVSGTGPCAG |
| | Accession Number | DFAFHKEGAFFLYDRLASTVIYRGTTFAEGVVAFLILP |
| | AAC57989.1) | QAKKDFFSSHPLREPVNATEDPSSGYYSTTIRYQATGF |
| | | GTNETEYLFEVDNLTYVQLESRFTPQFLLQLNETRYTS |
| | | GKRSNTTGKLIWKVNPEIDTTIGEWAFWETKKNLTRK |
| | | IRSEELSFTAVSNRAKNISGQSPARTSSDPGTNTTTEDH |
| | | KIMASENSSAMVQVHSQGREAAVSHLTTLATISTSLRP |
| | | PITKPGPDNSTHNTPVYKLDISEATQVEQHHRRTDNAS |
| | | TTSDTPPATTAAGPLKAENTNTSKGTDLLDPATTTSPQ |
| | | NHSETAGNNNTHHQDTGEESASSGKLGLITNTIAGVA |
| | | GLITGGRRTRREAIVNAQPKCNPNLHYWTTQDEGAAI |
| | | GLAWIPYFGPAAEGIYIEGLMHNQDGLICGLRQLANET |
| | | TQALQLFLRATTELRTFSILNRKAIDFLLQRWGGTCHI |
| | | LGPDCCIEPHDWTKNITDKIDQIIHDFVDKTLPDQGDN |
| | | DNWWTGWRQWIPAGIGVTGVIIAVIALFCICKFVF |
| 4 | Nucleic acid | ATGGGTGTTACAGGAATATTGCAGTTACCTCGTGAT |
| | sequence for Ebola | CGATTCAAGAGGACATCATTCTTTCTTTGGGTAATT |
| | virus glycoprotein | ATCCTTTTCCAAAGAACATTTTCCATCCCACTTGGA |
| | synthesized as a | GTCATCCACAATAGCACATTACAGGTTAGTGATGTC |
| | result of RNA | GACAAACTGGTTTGCCGTGACAAACTGTCATCCACG |
| | editing wherein an | AATCAATTGAGATCAGTTGGACTGAATCTCGAAGG |
| | additional A | GAATGGAGTGGCAACTGACGTGCCATCTGCAACTA |
| | residue is inserted | AAAGATGGGGCTTCAGGTCCGGTGTCCCACCAAAA |
| | during | GTGGTCAATTATGAAGCTGGTGAATGGGCTGAAAA |
| | transcription | CTGCTACAATCTTGAAATCAAAAAACCTGACGGGA |
| | (GenBank | GTGAGTGTCTACCAGCAGCGCCAGACGGGATTCGG |
| | Accession Number | GGCTTCCCCCGGTGCCGGTATGTGCACAAAGTATCA |
| | U77384) | GGAACGGGACCGTGTGCCGGAGACTTTGCCTTCCAC |
| | | AAAGAGGGTGCTTTCTTCCTGTATGATCGACTTGCT |
| | | TCCACAGTTATCTACCGAGGAACGACTTTCGCTGAA |
| | | GGTGTCGTGGCATTTCTGATACTGCCCCAAGCTAAG |
| | | AAGGACTTCTTCAGCTCACACCCCTTGAGAGAGCCG |
| | | GTCAATGCAACGGAGGACCCGTCTAGTGGCTACTAT |
| | | TCTACCACAATTAGATATCAGGCTACCGGCTTTGGA |
| | | ACCAATGAGACAGAGTATTTGTTCGAGGTTGACAAT |
| | | TTGACCTACGTCCAACTTGAATCAAGATTCACACCA |
| | | CAGTTTCTGCTCCAGCTGAATGAGACAAGATATACA |
| | | AGTGGGAAAAGGAGCAATACCACGGGAAAACTAAT |
| | | TTGGAAGGTCAACCCCGAAATTGATACAACAATCG |
| | | GGGAGTGGGCCTTCTGGGAAACTAAAAAAACCTCA |
| | | CTAGAAAAATTCGCAGTGAAGAGTTGTCTTTCACAG |
| | | CTGTATCAAACAGAGCCAAAAACATCAGTGGTCAG |
| | | AGTCCGGCGCGAACTTCTTCCGACCCAGGGACCAA |
| | | CACAACAACTGAAGACCACAAAATCATGGCTTCAG |
| | | AAAATTCCTCTGCAATGGTTCAAGTGCACAGTCAAG |
| | | GAAGGGAAGCTGCAGTGTCGCATCTGACAACCCTT |
| | | GCCACAATCTCCACGAGTCTTCGACCCCCCATAACC |
| | | AAACCAGGTCCGGACAACAGCACCCACAATACACC |
| | | CGTGTATAAACTTGACATCTCTGAGGCAACTCAAGT |
| | | TGAACAACATCACCGCAGAACAGACAACGCCAGCA |
| | | CAACCTCCGACACTCCCCCCGCCACGACCGCAGCCG |
| | | GACCCCTAAAAGCAGAGAACACCAACACGAGCAAG |
| | | GGCACTGACCTCCTGGACCCCGCCACCACAACAAG |
| | | TCCCCAAAACCACAGCGAGACCGCTGGCAACAACA |
| | | ACACTCATCACCAAGATACCGGAGAAGAGAGTGCC |
| | | AGCAGCGGGAAGCTAGGCTTAATTACCAATACTATT |
| | | GCTGGAGTCGCAGGACTGATCACAGGCGGGAGAAG |
| | | AACTCGAAGAGAAGCAATTGTCAATGCTCAACCCA |
| | | AATGCAACCCTAATTTACATTACTGGACTACTCAGG |
| | | ATGAAGGTGCTGCAATCGGACTGGCCTGGATACCAT |
| | | ATTTCGGGCCAGCAGCCGAGGGAATTTACATAGAG |
| | | GGGCTGATGCACAATCAAGATGGTTTAATCTGTGGG |
| | | TTGAGACAGCTGGCCAACGAGACGACTCAAGCTCT |
| | | TCAACTATTCCTGAGAGCCACAACCGAGCTACGCAC |
| | | CTTTTCAATCCTCAACCGTAAGGCAATTGATTTCTTG |
| | | CTGCAGCGATGGGGCGGCACATGCCACATTTTGGG |
| | | ACCGGACTGCTGTATCGAACCACATGATTGGACTAA |
| | | GAACATAACGGACAAAATTGATCAGATTATTCATG |
| | | ATTTTGTTGATAAAACCCTTCCGGACCAGGGGGACA |
| | | ATGACAATTGGTGGACAGGATGGAGACAGTGGATA |
| | | CCGGCAGGTATTGGAGTTACAGGCGTTATAATTGCA |
| | | GTTATCGCTTTATTCTGTATATGCAAATTTGTCTTTT |
| | | AG |
| 5 | Amino acid | MNYIPTQTFYGRRWRPRPAFRPWQVSMQPTPTMVTP |
| | sequence for Ross | MLQAPDLQAQQMQQLISAVSALTTKQNVKAPKGQRQ |
| | River Structural | KKQQKPKEKKENQKKKPTQKKKQQQKPKPQAKKKK |
| | polyprotein (P130) | PGRRERMCMKIENDCIFEVKLDGKVTGYACLVGDKV |
| | that contains: Coat | MKPAHVKGTIDNPDLAKLTYKKSSKYDLECAQIPVH |
| | protein C (Capsid | MKSDASKYTHEKPEGHYNWHHGAVQYSGGRFTIPTG |
| | protein C); Spike | AGKPGDSGRPIFDNKGRVVAIVLGGANEGARTALSVV |
| | glycoprotein E3; | TWTKDMVTRVTPEGTEEWSAALMMCILANTSFPCSSP |
| | Spike glycoprotein | PCYPCCYEKQPEQTLRMLEDNVNRPGYYELLEASMT |
| | E2; 6 kDa peptide; | CRNRSRHRRSVTEHFNVYKATRPYLAYCADCGDGYF |
| | and Spike | CYSPVAIEKIRDEAPDGMLKIQVSAQIGLDKAGTHAHT |
| | glycoprotein E1 | KIRYMAGHDVQESKRDSLRVYTSAACSIHGTMGHFIV |
| | (GenBank | AHCPPGDYLKVSFEDADSHVKACKVQYKHDPLPVGR |
| | Accession Number | EKFVVRPHFGVELPCTSYQLTTAPTDEEIDMHTPPDIP |
| | P08491) | DRTLLSQTAGNVKITAGGRTIRYNCTCGRDNVGTTST |
| | | DKTINTCKIDQCHAAVTSHDKWQFTSPFVPRADQTAR |
| | | RGKVHVPFPLTNVTCRVPLARAPDVTYGKKEVTLRLH |
| | | PDHPTLFSYRSLGAEPHPYEEWVDKESERIIPVTEEGIE |
| | | YQWGNNPPVRLWAQLTTEGKPHGWPHEIIQYYYGLY |
| | | PAATIAAVSGASLMALLTLAATCCMLATARRKCLTPY |
| | | ALTPGAVVPLTLGLLCCAPRANAASFAETMAYLWDE |
| | | NKTLFWMEFAAPAAALALLACCIKSLICCCKPFSFLVL |
| | | LSLGASAKAYEHTATIPNVVGFPYKAHIERNGFSPMTL |
| | | QLEVVETSWEPTLNLEYITCEYKTVVPSPFIKCCGTSEC |
| | | SSKEQPDYQCKVYTGVYPFMWGGAYCFCDSENTQLS |
| | | EAYVDRSDVCKHDHASAYKAHTASLKATIRISYGTIN |
| | | QTTEAFVNGEHAVNVGGSKFIFGPISTAWSPFDNKIVV |
| | | YKDDVYNQDFPPYGSGQPGRFGDIQSRTVESKDLYAN |
| | | TALKLSRPSPGVVHVPYTPTPSGFKYWLKEKGSSLNT |
| | | KAPFGCKIKTNPVRAMDCAVGSIPVSMDIPDSAFTRV |
| | | VDAPAVTDLSCQVVVCTHSSDFGGVATLSYKTDKPG |
| | | KCAVHSHSNVATLQEATVDVKEDGKVTVHFSTASAS |
| | | PAFKVSVCDAKTTCTAACEPPKDHIVPYGASHNNQVF |
| | | PDMSGTAMTWVQRLASGLGGLALIAVVVLVLVTCIT |
| | | MRR |
| 6 | Amino acid | EHTATIPNVVGFPYKAHIERNGFSPMTLQLEVVETSLE |
| | sequence for Ross | PTLNLEYITCEYKTVVPSPFIKCCGTSECSSKEQPDYQC |
| | River Virus E1 | KVYTGVYPFMWGGAYCFCDSENTQLSEAYVDRSDVC |
| | protein | KHDHASAYKAHTASLKATIRISYGTINQTTEAFVNGEH |
| | | AVNVGGSKFIFGPISTAWSPFDNKIVVYKDDVYNQDF |
| | | PPYGSGQPGRFGDIQSRTVESKDLYANTALKLSRPSPG |
| | | VVHVPYTQTPSGFKYWLKEKGSSLNTKAPFGCKIKTN |
| | | PVRAMDCAVGSIPVSMDIPDSAFTRVVDAPAVTDLSC |
| | | QVVVCTHSSDFGGVATLSYKTDKPGKCAVHSHSNVA |
| | | TLQEATVDVKEDGKVTVHFSTASASPAFKVSVCDAKT |
| | | TCTAACEPPKDHIVPYGASHNNQVFPDMSGTAMTWV |
| | | QRLASGLGGLALIAVVVLVLVTCITMRR |
| 7 | Nucleic acid | GAGCACACAGCCACAATTCCGAATGTGGTGGGGTT |
| | sequence for Ross | CCCGTATAAGGCTCACATTGAAAGGAATGGCTTCTC |
| | River Virus E1 | GCCCATGACTCTGCAGCTTGAAGTGGTGGAGACAA |
| | protein | GCTTGGAACCCACACTTAACCTGGAGTACATTACCT |
| | | GCGAATACAAGACGGTGGTCCCTTCGCCATTCATCA |
| | | AATGTTGCGGAACATCAGAATGCTCATCCAAGGAG |
| | | CAGCCAGACTACCAATGCAAGGTGTACACGGGTGT |
| | | ATACCCATTCATGTGGGGTGGAGCCTACTGTTTCTG |
| | | CGACTCCGAGAACACGCAGCTCAGCGAGGCCTATG |
| | | TCGACAGGTCAGACGTTTGCAAACATGATCACGCAT |
| | | CGGCCTACAAGGCACACACGGCCTCTCTAAAAGCA |
| | | ACAATCAGGATCAGTTATGGCACCATCAACCAGAC |
| | | CACCGAGGCCTTCGTTAATGGTGAACACGCGGTCA |
| | | ACGTGGGCGGAAGCAAGTTCATCTTTGGACCGATCT |
| | | CAACAGCTTGGTCACCGTTCGACAATAAAATTGTCG |
| | | TGTATAAAGATGATGTCTACAACCAGGACTTCCCAC |
| | | CCTACGGATCAGGCCAGCCGGGTAGATTCGGAGAC |
| | | ATTCAGAGCAGGACAGTGGAGAGCAAAGACTTGTA |
| | | TGCCAACACGGCCCTAAAACTCTCAAGACCATCACC |
| | | CGGGGTTGTGCATGTGCCATACACGCAGACACCATC |
| | | CGGATTTAAATATTGGCTGAAGGAGAAAGGATCTTC |
| | | ATTGAATACAAAGGCCCCTTTTGGCTGCAAGATAAA |
| | | GACCAATCCAGTCAGAGCCATGGATTGTGCAGTTGG |
| | | CAGTATACCTGTGTCGATGGACATACCTGACAGTGC |
| | | ATTCACACGAGTGGTAGATGCCCCGGCTGTAACAG |
| | | ACCTGAGCTGCCAGGTAGTGGTCTGTACACACTCCT |
| | | CCGATTTCGGAGGAGTTGCCACATTGTCTTACAAAA |
| | | CGGACAAACCCGGCAAGTGCGCTGTCCACTCACATT |
| | | CCAACGTCGCAACGTTGCAAGAGGCGACGGTGGAT |
| | | GTCAAGGAGGATGGCAAGGTCACAGTGCACTTTTCC |
| | | ACGGCGTCCGCCTCCCCGGCCTTCAAAGTGTCCGTC |
| | | TGTGACGCAAAAACAACGTGCACGGCGGCGTGCGA |
| | | GCCTCCAAAAGACCACATCGTCCCTTATGGGGCGA |
| | | GCCATAACAACCAGGTCTTTCCGGACATGTCAGGA |
| | | ACTGCGATGACGTGGGTGCAGAGGCTGGCCAGTGG |
| | | GTTAGGTGGGCTGGCTCTCATCGCGGTGGTTGTGCT |
| | | GGTCTTGGTAACCTGCATAACAATGCGTCGG |
| 8 | Amino acid | MSAALMMCILANTSFPCSSPPCYPCCYEKQPEQTLRM |
| | sequence for Ross | LEDNVNRPGYYELLEASMTCRNRSRHRRSVTEHFNV |
| | River Virus E3-E2 | YKATRPYLAYCADCGDGYFCYSPVAIEKIRDEASDGM |
| | protein | LKIQVSAQIGLDKAGTHAHTKIRYMAGHDVQESKRDS |
| | | LRVYTSAACSIHGTMGHFIVAHCPPGDYLKVSFEDAD |
| | | SHVKACKVQYKHDPLPVGREKFVVRPHFGVELPCTSY |
| | | QLTTAPTDEEIDMHTPPDIPDRTLLSQTAGNVKITAGG |
| | | RTIRYNCTCGRDNVGTTSTDKTINTCKIDQCHAAVTSH |
| | | DKWQFTSPFVPRADQTARRGKVHVPFPLTNVTCRVPL |
| | | ARAPDVTYGKKEVTLRLHPDHPTLFSYRSLGAEPHPY |
| | | EEWVDKFSERIIPVTEEGIEYQWGNNPPVRLWAQLTTE |
| | | GKPHGWPHEIIQYYYGLYPAATIAAVSGASLMALLTL |
| | | AATCCMLATARRKCLTPYALTPGAVVPLTLGLLCCAP |
| | | RANA |
| 9 | Nucleic acid | ATGTCTGCCGCGCTGATGATGTGTATCCTTGCCAAC |
| | sequence for Ross | ACCTCTTTCCCCTGCTCATCACCTCCCTGCTACCCCT |
| | River Virus E3-E2 | GCTGCTACGAAAAACAGCCAGAACAGACACTGCGG |
| | protein | ATGCTGGAAGACAATGTGAATAGACCAGGGTACTA |
| | | TGAGCTACTGGAAGCGTCCATGACATGCAGAAACA |
| | | GATCACGCCACCGCCGTAGTGTAACAGAGCACTTC |
| | | AATGTGTATAAGGCTACTAGACCGTACTTAGCGTAT |
| | | TGCGCTGACTGTGGGGACGGGTACTTCTGCTATAGC |
| | | CCAGTTGCTATCGAGAAGATCCGAGATGAGGCGTCT |
| | | GACGGCATGCTCAAGATCCAAGTCTCCGCCCAAAT |
| | | AGGTCTGGACAAGGCAGGTACCCACGCCCACACGA |
| | | AGATCCGATATATGGCTGGTCATGATGTTCAGGAAT |
| | | CTAAGAGAGATTCCTTGAGGGTGTACACGTCCGCAG |
| | | CGTGCTCTATACATGGGACGATGGGACACTTCATCG |
| | | TCGCACATTGTCCGCCAGGCGACTACCTCAAGGTTT |
| | | CGTTCGAGGACGCAGATTCACACGTGAAGGCATGT |
| | | AAGGTCCAATACAAGCACGACCCATTGCCGGTGGG |
| | | TAGAGAGAAGTTCGTGGTTAGACCCCACTTTGGCGT |
| | | AGAGCTGCCATGCACCTCATACCAGCTGACAACAG |
| | | CTCCCACCGACGAGGAGATCGACATGCACACACCG |
| | | CCAGATATACCGGATCGCACCCTGCTATCACAGACG |
| | | GCGGGCAACGTCAAAATAACAGCAGGCGGCAGGAC |
| | | TATCAGGTACAATTGTACCTGTGGCCGTGACAACGT |
| | | AGGCACTACCAGTACTGACAAGACCATCAACACAT |
| | | GCAAGATTGACCAATGCCATGCTGCCGTTACCAGCC |
| | | ATGACAAATGGCAATTTACCTCTCCATTTGTTCCCA |
| | | GGGCTGATCAGACAGCTAGGAGGGGCAAAGTGCAT |
| | | GTTCCATTCCCTTTGACTAACGTCACCTGCCGAGTG |
| | | CCGTTGGCTCGAGCGCCGGATGTCACCTATGGTAAG |
| | | AAGGAGGTGACCCTGAGATTACACCCAGATCATCC |
| | | GACGCTCTTCTCCTATAGGAGTTTAGGAGCCGAACC |
| | | GCACCCGTACGAGGAGTGGGTTGACAAGTTCTCTGA |
| | | GCGCATCATCCCAGTGACGGAAGAAGGGATTGAGT |
| | | ACCAGTGGGGCAACAACCCGCCGGTCCGCCTATGG |
| | | GCGCAACTGACGACCGAGGGCAAACCCCATGGCTG |
| | | GCCACATGAAATCATTCAGTACTATTATGGACTATA |
| | | CCCCGCCGCCACCATTGCCGCAGTATCCGGGGCGA |
| | | GTCTGATGGCCCTCCTAACTCTAGCGGCCACATGCT |
| | | GCATGCTGGCCACCGCGAGGAGAAAGTGCCTAACA |
| | | CCATACGCCTTGACGCCAGGAGCGGTGGTACCGTTG |
| | | ACACTGGGGCTGCTTTGCTGCGCACCGAGGGCGAA |
| | | CGCA |

The present invention is illustrated by the following examples. It is to be understood that the particular examples, materials, amounts, and procedures are to be interpreted broadly in accordance with the scope and spirit of the invention as set forth herein.

### EXAMPLE I

### Preparation of Pseudotyped Viral Particles Having Moloney Murine Leukemia Virus (Mu-LV or MMLV) Capsid Proteins and a Glycoprotein in which the O-Glycosylation (Mucin) Domain is Deleted

Cell lines and culture conditions: The human kidney cell line 293 (ATCC Number CRL-1573), the mouse embryo cell line NIH 3T3 (CRL-1658), and the 293T-derived ΦNX (second generation retroviral packaging cells) and gpnlslacZ cell lines were cultured in Dulbecco's minimal essential medium (DMEM) containing 10% heat inactivated fetal bovine serum, 2 mM glutamine, 100 units Penicillin G, and 100 μg/ml streptomycin sulfate, with or without 0.25 μg/ml amphotericin B (growth medium). The gpnlslacZ cells produce envelope protein-deficient replication-incompetent Mo-MuLV particles carrying MFG.S-nlslacZ, a retroviral vector encoding a nuclear localizing β-galactosidase.

Plasmids and site-directed mutagenesis: A modified version of the plasmid pTM1 was used in transient expression studies of glycoprotein (GP) sequences using a vaccinia virus-T7 RNA polymerase (VV-T7) system. The pTM1 vector was modified to remove an ATG codon (within an Ncol site) at the beginning of the multiple cloning site by Ncol digestion, mungbean nuclease treatment, and ligation of the blunt-ended DNA. This vector, pTM1(ΔNcol), was used to subclone the entire Ebola virus GP open reading frame (ORF). The glycoprotein ORF was cleaved from the plasmid pGEM-EMGP1 by digestion with BamHI and Dral, and the fragment isolated and directionally ligated into the pTMI (ΔNcol) vector cleaved with BamHI and Stul. The resulting clone, pTM1(ΔNcol)-GP, was used as the target DNA for all site-directed mutagenesis reactions. This clone encodes a glycoprotein sequence that differs from the wild-type amino acid sequence in a single residue within the membrane spanning sequence (1662V), and for comparative purposes will be referred to as “wild-type sequence”. This mutation is present in the original pGEM3Zf(−)-GP clone, but does not appear to affect the processing or function of the glycoprotein. Glycoprotein residue numbering commences with the methionine of the signal sequence and is continuous through the glycoprotein, and GP₂ sequences.

The glycoprotein clone in which the mucin region was deleted (Δ309-489) was generated from two PCR clones linked by an Xbal restriction site, which resulted in the replacement of the mucin sequence with two residues (serine-arginine). Mutations in isolated plasmid clones were identified by direct sequencing of mini-prep DNA using dye-terminator cycle sequencing reactions (ABI) analyzed on either an ABI 373 or 377 sequencer. Large-scale preparations for each type of mutated plasmid DNA were made using commercial kits (Promega Corp., Madison, Wis. or 5 Prime→3 Prime, Inc., Boulder, Colo.). The DNA was quantified by UV₂₆₀ absorbance readings, and then stored at −70° C. until needed. The coding region (BamHI/SalI fragments) from the plasmid pTM1(ΔNcol)-GP and mutated versions of this DNA were separately ligated into the BamHI/Xhol polylinker sites of the vector pcDNA3 (Invitrogen, Carlsbad, Calif.), cloned in E. coli, and plasmid DNA was isolated for use in pseudotyping studies.

Retrovirus pseudotyping and virus transduction assays: Pseudotyped retrovirus particles consisting of the Moloney Murine Leukemia Virus (MuLV) cores and the Ebola glycoprotein in their envelopes were produced by transfecting wild-type or mutated plasmid DNA into gpnlslacZ cells (Sharkey et al., J. Virol., 75:2653 (2001)). Virus transduction of 13-galactosidase activity into NIH 3T3 cells was determined as previously described (Sharkey et al., J. Virol., 75:2653 (2001)). All data presented are the average of the results of at least three experiments.

Immunoblot analysis of Ebola virus glycoprotein expression, processing, and incorporation into pseudotyped retroviruses: Medium from transfected ΦNX cells containing recombinant retroviruses were passed through a 0.45 μm filter and centrifuged through a 30% sucrose cushion at 25,000 RPM in a Beckman 50.2-Ti rotor in a Beckman SS-71 centrifuge. The fluid was aspirated from centrifuge tubes and discarded, and the virus pellet was suspended in 100 μl of RIPA buffer (140 mM NaCl, 10 mM Tris HCl pH 8.0, 5 mM EDTA, 1% Na deoxycholate, 1% Triton X-100, 0.1% SDS). Cells were treated with lysis buffer (50 mM Tris HCl pH 8.0, 5 mM EDTA, 150 mM NaCl, 1% Triton X-100), and the cell lysates were then centrifuged in a microcentrifuge at 16,100× gravity (g) for ten minutes. The proteins in the cell lysate and the suspended viral pellet were each precipitated with a final concentration of 4% TCA for two minutes. The precipitated proteins were then centrifuged at 16,100×g in a microcentrifuge for ten minutes. The supernatant fluid was aspirated and discarded. The pellet was suspended in an equal volume of 1 molar Tris[hydroxymethyl)-aminomethane and vortexed vigorously. Proteins whose glycosylation was analyzed were treated sequentially with PNGase F, which removes N-linked glycosylation, and with both Sialidase A and Endo-Oglycosidase (ProZyme, Inc., San Leandro, Calif.), which together remove O-linked glycosylation, following protocols provided by the supplier. The pellet suspension was then mixed with ⅙ the volume of 300 mM Tris pH 6.8, 60% glycerol (w/v), 4% SDS (w/v), 0.0012% bromophenol blue (w/v), 6% 2-mercaptoethanol (v/v) and boiled for 5 minutes. Equal amounts of protein, as determined by the Bradford assay, were separated by SDS-PAGE (8.5% acrylamide), and electrophoretically blotted onto nitrocellulose membranes. Membrane blots were immersed in reaction buffer (20 mM Tris, 137 mM NaCl, 0.1% Tween 20, pH 7.6) containing 1% bovine serum albumin and incubated overnight at 4° C. Blots were incubated in reaction buffer containing a rabbit-anti-Ebola SGP/GP diluted 1:1000 for 1 hour at room temperature, washed 3 times in reaction buffer, and reacted with a Goat anti-rabbit-horseradish peroxidase conjugate (diluted 1:20,000 in reaction buffer) for 30 minutes at room temperature. Membranes were washed as before, then treated with a commercial chemiluminescent substrate solution (Amersham Pharmacia Biotech, Piscataway, N.J.), according to the protocols provided by the manufacturer. Specific reactivity to the glycoprotein was visualized by exposing treated blots to X-ray film.

Pseudotyped Retroviruses Bearing GPs with Altered Glycosylation.

Processing and viral incorporation of an Ebola glycoprotein in which the O-glycosylation region was deleted (Δ309-489 glycoprotein) was greatly enhanced. Migration of the mature GP₁, GP₀ (the glycosylated but uncleaved glycoform) GPₚᵣₑ (the N-glycosylated but not O-glycosylated uncleaved glycoform) and deglycosylated GP₁ and GPₚᵣₑ forms of the wild-type GP and of the GP₁, GP₀ and deglycosylated GP₁, and GP₀ forms of the Δ309-489 GP is indicated. There was also a corresponding increase of 696±142% in transduction by the Δ309-489 glycoprotein pseudotyped viruses as compared to wild type. The absence of an increase in the mobility of the Δ309-489 GP upon sialadase A and endo-O-glycosidase treatment confirmed that the region of O-glycosylation of the glycoprotein has been removed. Migration of the mature GP₁ of the wild-type and Δ309-489 GPs is indicated. In this experiment a glycosylated serum protein possessing a mobility intermediate between those of the wild-type and Δ309-489 GP₁s was detected. The heterogeneous mobility of the PNGaseF-treated proteins is indicative of incomplete removal of N-glycosylation.

The effect of deleting the O-glycosylation region of GP₁ (Δ309-489) on expression and transduction were striking. This segment, which is rich in proline, serine, and threonine residues is the most variable among the Ebola GPs. Elimination of this mucin-like domain resulted in enhanced GP processing and incorporation into retroviral particles and consequently higher levels of transduction by the pseudotyped retroviruses. It is possible that the wild-type GP is retained in the Golgi apparatus until all of the serines and threonine residues in the mucin region are modified. It is thought that elimination of this segment may permit more rapid transit through the Golgi apparatus and higher levels of processing to GP₁ and GP₂ and of cell-surface expression. Increased viral incorporation may also result from a diminution of GP toxicity. It has been reported that the deletion of the O-glycosylation region reduces the cytopathic effects of Ebola virus GP expression. Expression of high levels of the wild-type Ebola GP might lead to exhaustion of the cellular glycosylation machinery, which is consistent with the present results and present interpretation.

### EXAMPLE II

### Preparation and Gene Transfer by Pseudotyped Viral Particles Having Feline Immunodeficiency Virus (FIV) Capsid Proteins and a Vesicular Stomatitis Virus G Protein (VSV-G), Marburg Virus (MRB) or Ross River Virus (RRV) Glycoprotein

Vector production: The second generation feline immunodeficiency virus (FIV) vector system was previously reported (Johnston et al., J. Virol., 73:4991 (1999)). Plasmid constructs consist of an FIV packaging construct with a deletion in the env gene and mutations in vif and orf2, an FIV vector construct expressing cytoplasmic E. coli β-galactosidase, eGFP or other nucleic acid sequences of interest, and an envelope plasmid in which the human CMV early gene promoter directs transcription of the Marburg envelope cDNA. The FIV packaging plasmid (pCFIVΔorf2Δvif) contains the FIV packaging signal, the gag and pol genes and the rev sequences. FIV rev is analogous to the HIV rev in enabling expression of late genes encoded by unspliced or singly spliced mRNAs containing the cis-acting Rev-responsive element (RRE). The proviral FIV 5′ LTR is replaced by the CMV promoter/enhancer and the 3′ LTR is replaced with the simian virus 40 polyadenylation signal. A deletion in the env gene and mutations in FIV accessory genes vif and orf2 render these sequences inactive without negatively affecting vector titer.

The FIV vector plasmids (based on pVETL) consist of the FIV 5′ and 3′ LTR sequences flanking a portion of the gag sequence including the packaging signal, a transgene cassette, and the Rev responsive element (RRE). The U3 region of the 5′FIV LTR is replaced with the CMV promoter. A CMV promoter-β-Gal expression plasmid, pCMVβgal, was generated by combining an XbaI/SalI fragment corresponding to the CMV promoter from pCMV-G and a SalI/SmaI fragment corresponding to the β-Gal gene from pSP6 β-GAL into pBlueScript SK(−). pTFIVLCβ, pTC/FLCβ, and pTC/FSCβ were then generated by insertion of the NotI/SmaI CMV-β-Gal expression cassette from pCMVβ gal into similarly digested pTFIVL, pTC/FL, and pTC/FS vector backbones, respectively. These constructs were renamed PTFIV_{L}Cβ, PVET_{L}Cβ, and pVET_{S}Cβ, respectively. A pCMVβgalCTE expression plasmid was used to generate an FIV expression vector containing the constitutive RNA transport element (CTE) from Mason-Pfizer monkey virus (MPMV). pCMVβgalCTE was constructed in part from pSK-CTE. pSK-CTE was generated by PCR amplification of the CTE with the primers CTEH5 and CTEH3, which harbor HindIII sites near their 5′ ends. The resulting PCR product was digested with HindIII and inserted into similarly digested pBlueScript SK(−) to generate pSK-CTE. pSK-CTE was then digested with SmaI and XhoI, and the insert was ligated into similarly digested pCMVβgal to generate pCMVβgalCTE. A NotI/XhoI fragment containing the CMVβgalCTE expression cassette from pCMVβgalCTE was then ligated into NotI/SalI-digested pTC/FL to create pTC/FLCβCTE (now referred to as pVET_{L}Cβ_{CTE}).

The VSV-G envelope plasmid, pCMV-G, encodes the VSV envelope glycoprotein (Yee et al., P.N.A.S. USA, 91:9564 (1994)). The pRRV-E2E1 plasmid encodes the full-length RRV envelope glycoprotein, E3-E3-6K-E1, which is processed proteolytically into the individual subunits. The region encoding the RRV envelope glycoproteins was amplified from pRR64, which contains the full-length cDNA of the RRV genome (Kuhn et al., Virology, 182:430 (1991)), using Taq DNA polymerase (Promega Corporation, Madison, Wis.) and two primers complementary to the viral cDNA at nucleotides 8376 and 11312. The amplified fragment, which contained the RRV E3-E2-6K-E1 coding region, was digested with the restriction endonucleases BamHI and XbaI and ligated into the BamHI and XbaI sites of pBacPac, a baculovirus expression vector (Clontech, Palo Alto, Calif.). The resulting plasmid was digested with BamHI and XbaI, and the fragment containing the RRV E3-E2-6K E1 coding region was ligated into the BamHI and XbaI sites in the pcDNA3 and pcDNA3.1/Zeo(+) mammalian expression vectors (Invitrogen, Carlsbad, Calif.). The resulting plasmids were designated pRRV-E2E1 and pRRV-E2E1A, respectively.

To construct a plasmid encoding the Marburg glycoprotein (SEQ ID NO:1, Genbank Accession Number Z12132), nucleotides 5931-8033 from the Marburg virus genome were cloned into the pSP72 plasmid (Promega, Madison, Wis.) under the control of the T7 promoter using SalI. The Xhol and EcoRI fragment of this plasmid was cloned into the Xhol and EcoRI polylinker sites of the mammalian expression vector pcDNA3. The amino acid sequence of an example of a Marburg virus glycoprotein is shown in FIG. 6, Table 1 and in SEQ ID NO:1.

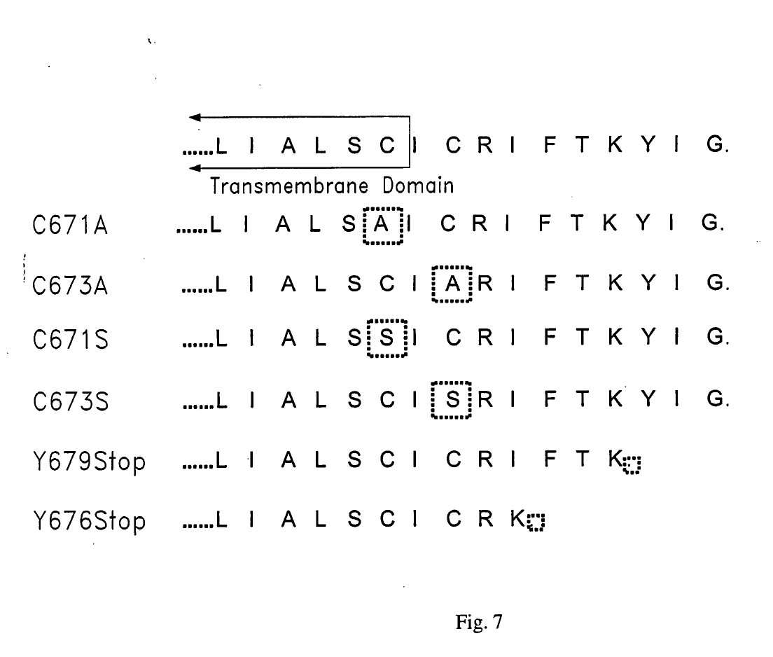

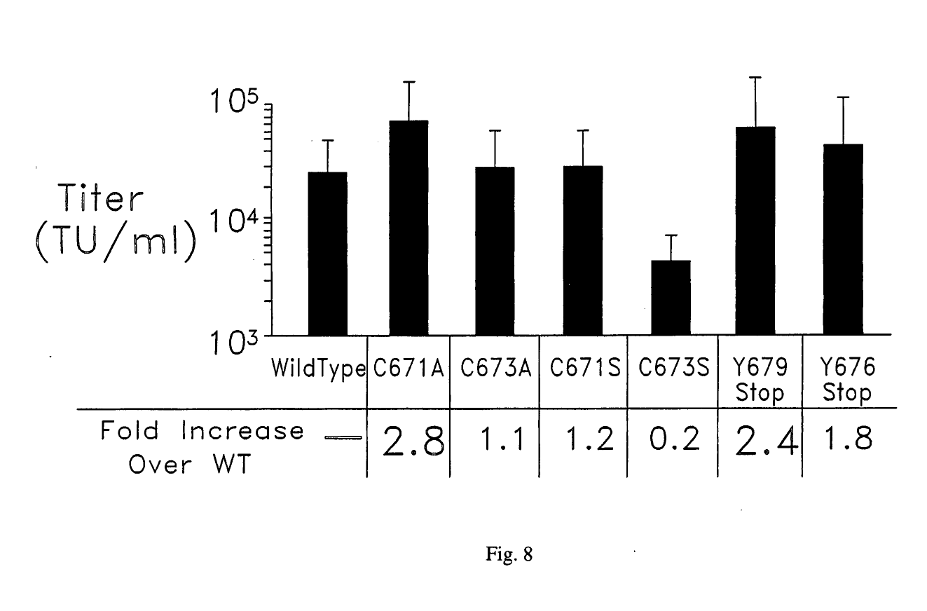

Mutants of the Marburg glycoprotein were also produced through use of standard mutagenesis methods. These mutants included Marburg glycoproteins in which the cysteine at amino acid position 671 was replaced with an alanine, valine, glycine, isoleucine, or leucine (FIG. 7, SEQ. ID. NOs: 10-16: top to bottom). Replacement of the cysteine at position 671 with an aliphatic, non-polar amino acid, caused the titer of FIV pseudotyped viral particles with the mutant Marburg glycoprotein to increase about 3-fold (FIG. 8). The Marburg glycoprotein was also mutated to truncate the carboxyl-terminus. Truncation of the Marburg virus glycoprotein with a Y679 stop mutation caused pseudotyped viruses containing the truncated glycoprotein to have at least a two-fold increase in virus titer production. In addition, truncation of the carboxyl-terminus of the Marburg glycoprotein at isoleucine 680 (I680Stop) to about phenylalanine 676 (F676Stop) (FIG. 7, SEQ ID NOs: 10-16; top to bottom)) resulted in about a 2-fold increase in the titer of FIV pseudotyped with the truncated glycoprotein (FIG. 8).

Pseudotyped FIV vector particles were generated by transient transfection of plasmid DNA into 293T cells plated 1 day prior to transfection at a density of 2.8×10⁶ per 10-cm-diameter culture dish (as described by Johnston et. al., J. Virol., 73:4991 (1999)). Three plasmid cotransfections were performed using packaging, envelope, and vector plasmids, followed by collection of supernatants and particle concentration by centrifugation. For each preparation, 750 ml of culture supernatant was centrifuged overnight at 7,400×g and resuspended in 3 ml of lactose buffer (19.5 mM Tris at pH 7.4, 37.5 mM NaCl, and 40 mg/ml lactose). Transduction titers before and after concentration were determined by measurement of X-Gal (5-bromo-4 chloro-β-indolyl-3-D-galactopyranoside)-positive cells in tranduced HT-1080 target cells and were expressed as tranducing units (TU)/ml.

Determination of β-Galactosidase Expression

For X-Gal staining of liver after intravenous vector injection, lobes were fixed in 2% paraformaldehyde-PBS overnight and then stained with X-Gal overnight at 4° C. The overall expression of β-galactosidase was first examined by stereo microscopy. The X-Gal-stained tissue was then embedded in paraffin, and 5-μm sections were cut at 50-μm intervals and counterstained with hematoxylin and eosin for quantification and histological examination. For X-Gal staining of brain and muscle sections, 10-mm sections on slides were incubated in X-Gal for 6 hours at 37° C., washed in PBS, and counterstained with neutral red. For X-Gal staining of lung, the lungs were removed, inflated with and submersed in 2% paraformaldehyde-PBS, and allowed to fix for 4 hours at 4° C. After fixation, the lungs were washed with PBS and inflated with X-Gal solution. The lungs were submersed in additional X-Gal and incubated overnight at 37° C. After X-Gal staining, the lungs were washed with PBS and paraffin embedded by a standard protocol, and 10-μm sections were collected. Sections were counterstained with nuclear fast red.

Immunostaining

To determine the cell types transduced after intrastriatal injections of RRV pseudotyped FIV, 10-μm brain sections were dually stained for β-galactosidase and glial fibrillary acidic protein (GFAP, a type II astrocyte-specific intermediate filament), or for β-galactosidase and 2′,3′-cyclic nucleotide-3′-phosphodiesterase (CNPase, an oligodendrocyte-myelin specific marker) and analyzed by confocal fluorescence microscopy. The antibodies used were polyclonal rabbit anti-β-galactosidase (Biodesign International, Saco, Me.), Cy3-conjugated mouse monoclonal anti-GFAP (Sigma), mouse monoclonal anti-CNPase (Sigma) Alexa 488-conjugated goat anti-rabbit IgG (Molecular Probes, Eugene, Oreg.) and lissamine-rhodamine-conjugated goat anti-mouse IgG (Jackson ImmunoResearch, West Grove, Pa.). Sections were blocked with 10% normal goat serum and 0.1% Triton X-100 in PBS for 1 hour at room temperature and then incubated with primary antibodies overnight at 4° C. in PBS with 3% bovine serum albumin and 0.1% Triton X-100. The sections were then washed, incubated with secondary antibodies for 2 hours at room temperature, washed, and coverslipped with gel mount. Using confocal microscopy, images from 0.3-μm-thick Z series were collected.

Gene Transfer to Lung Tissue

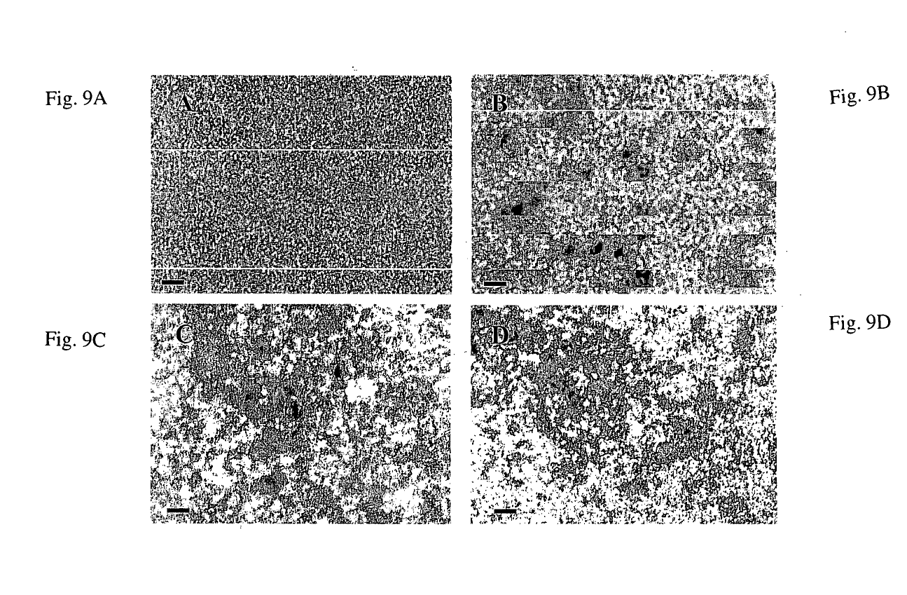

In vitro: To transduce differentiated human epithelia, the pseudotyped FIV vector was mixed with cell culture medium to a final volume of 100 μl (multiplicity of infection (MOI) about 10). This mixture was applied to either the apical surface or the basal surface of primary cultures of human airway epithelia as described previously (Wang et. al., J. Virol., 104:R49 (1999)). To enhance transduction from the apical surface, vector was mixed at a 1:1 (vol/vol) ratio with 12 mM EGTA HEPES/saline solution (pH 7.3), and applied apically for 4 hours as previously reported for Murine leukemia virus vectors (Wang et. al., J. Virol., 104:R49 (1999)). The results are shown in FIGS. 9A-9D. The pseudotyped FIV vector was effective in transducing cells when applied to either the basal or apical surface of the cells. In contrast, the VSV-G control could not transduce the cells when applied to the apical surface.

In vivo: For tracheal gene transfer, adult New Zealand white rabbits were anesthetized with 32 mg/kg ketamine, 5.1 mg/kg xylazine and 0.8 mg/kg acepromazine intramuscularly, a ventral midline incision made and tracheotomy was performed. An approximately 1.5 cm tracheal segment cephalad to the tracheotomy was isolated and cannulated on each end with PE 330 tubing (Clay Adams, Becton Dickinson, Franklin Lakes, N.J.). The tracheal segment was rinsed and then filled with a FIV-β-gal vector solution. The vector solution was left in place for 45 minutes, then the cannulae were removed and the incisions closed. Five days or six weeks later, the tissues were studied for β-galactosidase expression. For lower airway gene transfer, a PE50 catheter was passed via the trachea until it lodged in a subsegmental bronchus. 200-600 μl of FIV-β-gal of various envelope pseudotypes was instilled. Five days later, the tissues are studied for β-galactosidase expression.

Gene Transfer to Brain

Six to 8 week old adult male C57BL/6 mice were used for gene transfer. Mice were anaesthetized and 5×10⁵ TU of the vectors were stereotactically injected into either the right lateral ventricle or the right striate, using a 26 gauge Hamilton syringe driven by a microinjector (Micro 1, World Precision Instruments, Sarasota, Fla.) at 0.5 μl per minute. For ventricular injections, 10 μl volumes were injected at coordinates 0.4 mm anterior, 1.0 mm lateral to bregma at 2 mm depth. For striatal injections, 5 μl volumes are injected at coordinates 0.4 mm rostral and 2 mm lateral to bregma, and at a 3 mm depth. A minimum of two independent experiments were done for each vector and injection site. At 3 weeks postinjection, mice were sacrificed and perfused with 2% formaldehyde in PBS. The brains were postfixed overnight at 4° C. and cryoprotected in 30% sucrose-PBS for 48 h at 4° C. The hemispheres were separated and blocked in O.C.T. (Sakura Finetek USA, Torrance, Calif.) by freezing in a dry ice-ethanol bath. Parasagittal cryosections (10 μm) were cut and placed on slides. Slides were stained with X-Gal or were dually stained with antibodies for immunofluorescent confocal analysis.

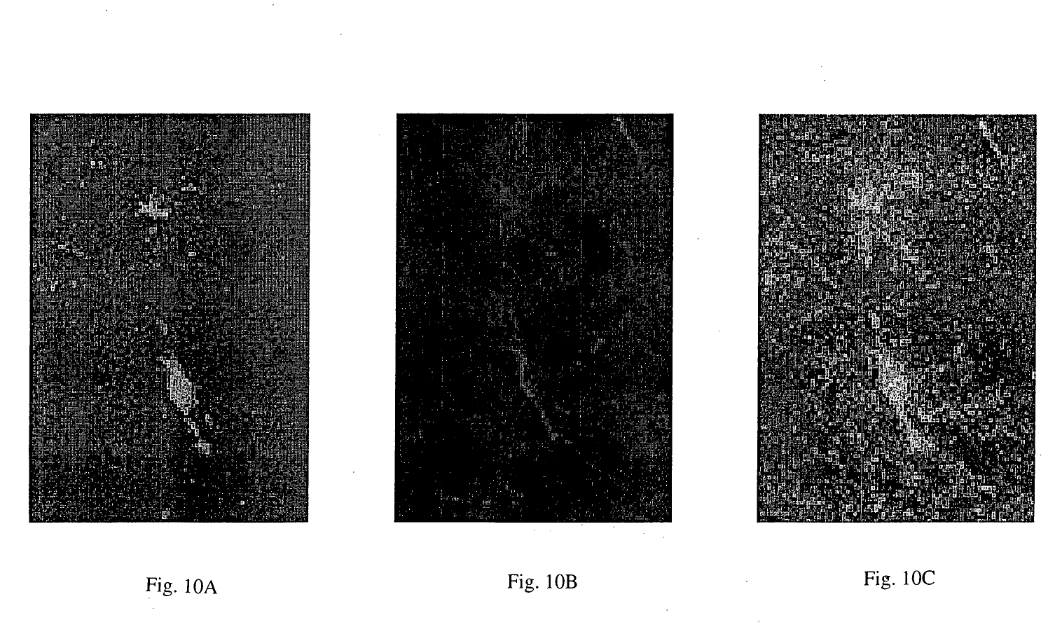

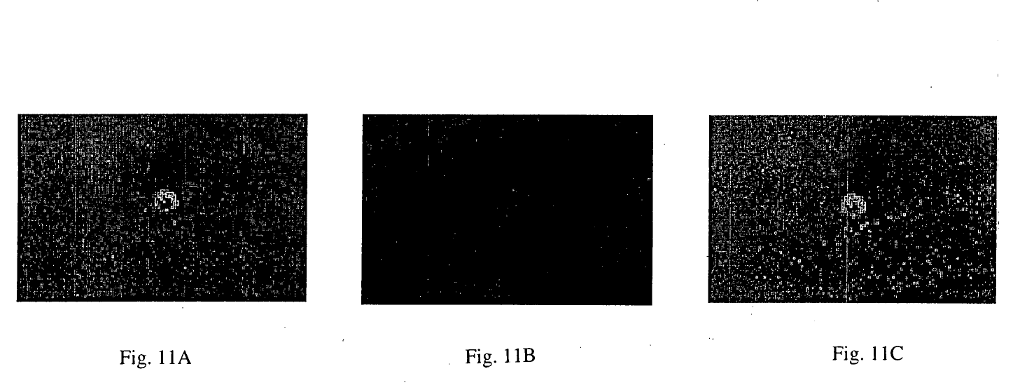

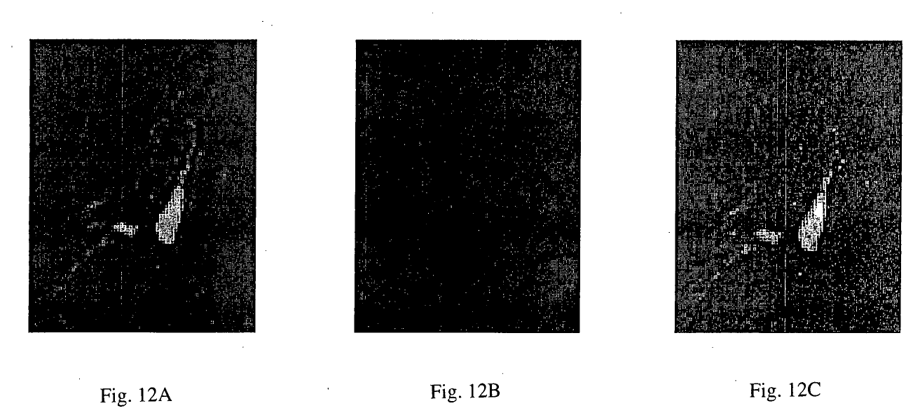

The feline immunodeficiency virus (FIV) pseudotyped with at least two different Ross River (RRV) viral glycoproteins was effective in transducing brain astrocytes (FIGS. 10A-10C). The presence of the marker protein GFAP confirmed that the brain glial cells were astrocytes. The FIV virus pseudotyped with RRV glycoproteins was also effective in transducing oligodendrocytes (FIGS. 11A-11C and 12A-12C). The presence of the marker protein CNPase confirmed that the brain glial cells were oligodendrocytes. The data in Table 2 confirms the selective transduction of astrocytes and oligodendrocytes (oligos) by the togaviral pseudotyped lentivirus as compared to other types of brain cells.

TABLE 2
Gene Transfer to Brain

| Cell Type | % Transduced |
|---|---|
| Neurons | 7.0 +/− 6.5% |
| Astrocytes | 56.5 +/− 17.2% |
| Microglia | 9.9 +/− 5.5% |
| Oligodendrocytes | 26.6% |

Gene Transfer to Liver The C57Bl/6 mice were intravenously injected via tail vein with FIV vector (total dose 1.3×10⁷ to 6×10⁷ IU), administrated over one or on two consecutive days (one injection/day). Controls received vector buffer. The injection volume was 0.4 ml. On days 1 and 7 postinjection, blood samples were obtained from the retro-orbital plexus and the serum samples assayed for the levels of glutamic oxalacetic transaminase (SGOT) and glutamic pyruvic transaminase (SGPT) using a transaminase assay kit (Sigma, St. Louis, Mo.) according to the manufacturer's instructions. At 3 weeks postinjection, the mice were sacrificed and perfused with cold phosphate-buffered saline (PBS). Samples of liver, spleen, kidney, lung, heart, and skeletal muscles (triceps) were harvested for X-Gal staining.

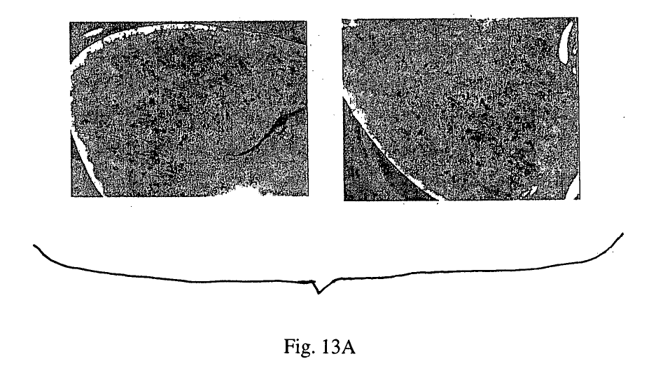

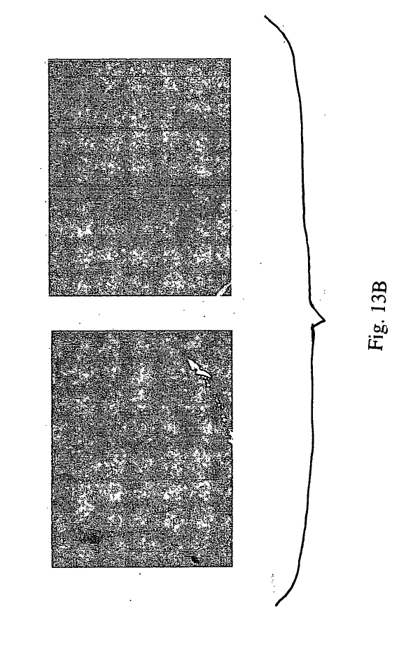

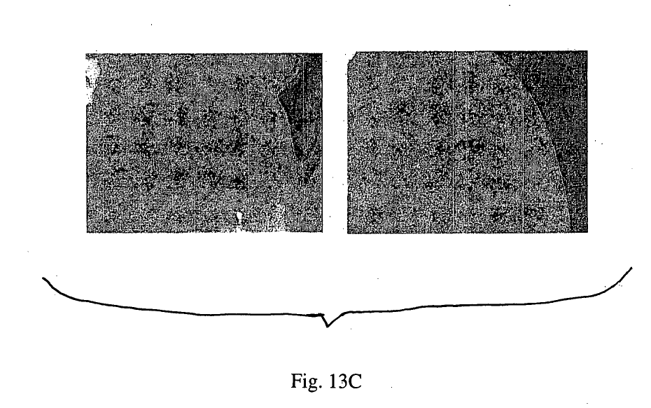

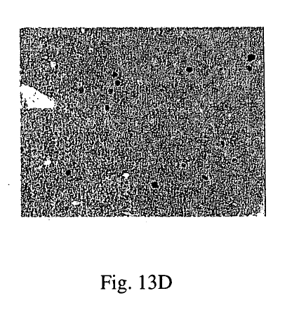

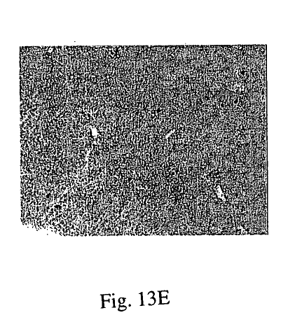

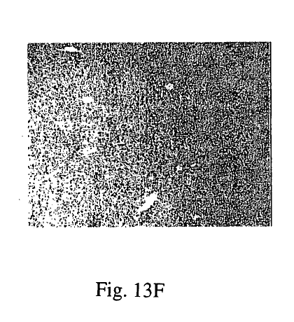

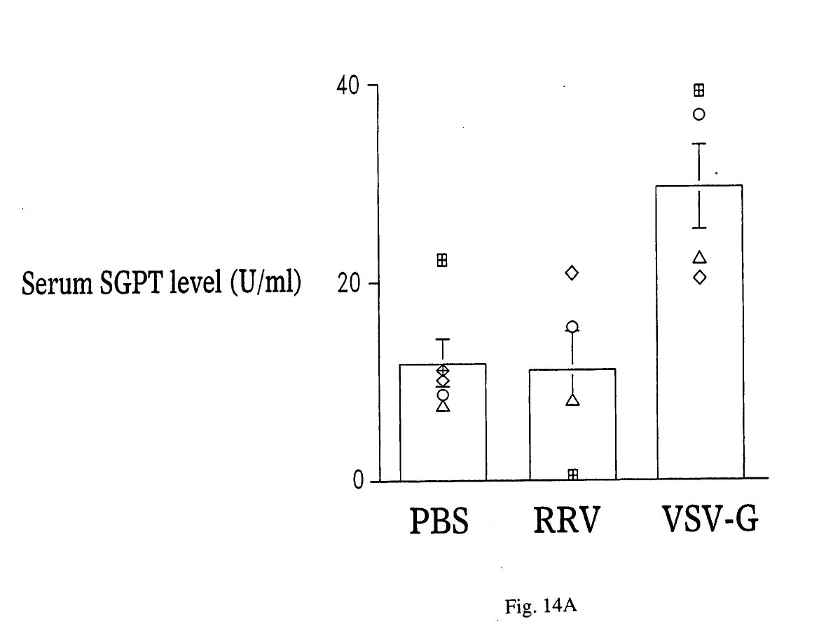

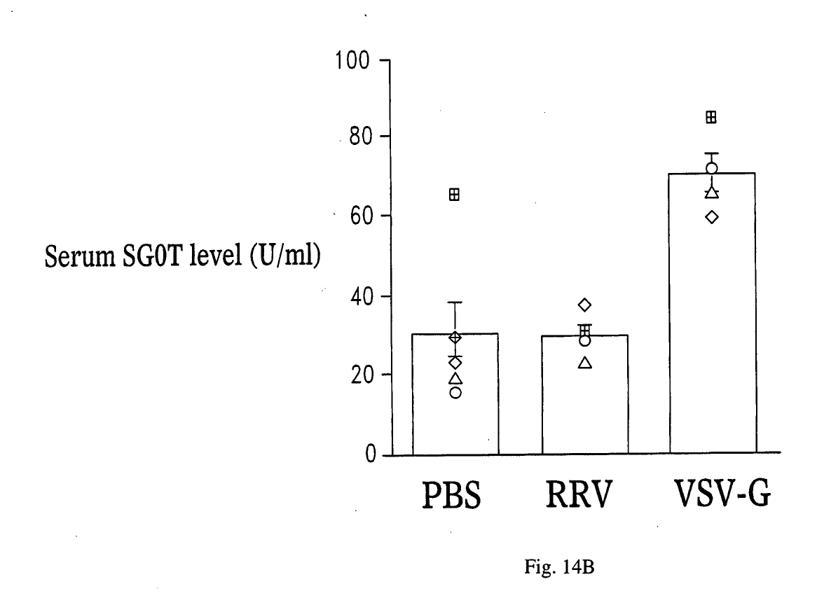

The pseudotyped viruses of the present invention were found to effectively transduce hepatocytes in vivo. FIGS. 13A-13F show that transduction with RRV-pseudotyped FIV is extensive throughout the liver (FIG. 13A), especially when compared to a VSV-G pseudotyped lentivirus control (FIG. 13B). Furthermore, the viruses of the present invention do not affect liver function as measured by SGOT and SGPT levels of treated livers (FIGS. 14A and 14B). This is in stark contrast to the VSV-G pseudotyped virus (FIGS. 14A and 14B), which has been reported to be toxic to a variety of cell types.

### EXAMPLE III

### Modified Ebola Virus Glycoproteins and Viral Particles Containing the Modified Ebola Virus Glycoproteins

Cell lines and culture conditions: The human kidney cell line 293 (ATCC CRL-1573), the mouse embryo cell line NIH 3T3 (CRL-1658), and the 293T cell-derived ΦNX cell line (second-generation retroviral packaging cells) (Grignani et al., Cancer Res., 58:14 (1998); Pear et al., P.N.A.S. USA, 90:8392 (1993); Swift et al., (1999) Rapid production of retroviruses for efficient gene delivery to mammalian cells using 293T cell-based systems, p. 10.17.14-10.17.29. In R. Coico (ed.), Current protocols in immunology, Suppl. 31. John Wiley & Sons, Inc., New York, N.Y.) and gpnlslacZ cell line were cultured in Dulbecco's minimal essential medium (DMEM) containing 10% heat-inactivated fetal bovine serum, 2 mM glutamine, 100 U of penicillin G, and 100 μg of streptomycin sulfate/ml, with or without 0.25 μg of amphotericin B/ml (growth medium). The gpnlslacZ cells produce envelope protein-deficient replication-incompetent Moloney murine leukemia virus (MuLV) particles carrying MFG.S-nlslacZ, a retroviral vector encoding β-galactosidase localized to the nucleus (Sharkey et al., J. Virol., 75:2653 (2001)).

Plasmids and site-directed mutagenesis: A modified version of plasmid pTM1 was used in transient expression studies of Ebola virus Zaire glycoprotein (GP) sequences with a vaccinia virus-T7 RNA polymerase (VV-T7) system (Elroy-Stein et al., P.N.A.S. USA, 86:6126 (1989)). The pTM1 vector was modified to remove an ATG codon (within an NcoI site) at the beginning of the multiple cloning site by NcoI digestion, mung bean nuclease treatment, and ligation of the blunt-ended DNA. The resulting vector, pTM1(ΔNcoI), was used to subclone the entire Ebola virus GP open reading frame (ORF). The GP ORF was cleaved from plasmid pGEM-EMGP1 (Sanchez et al., Virus Res., 29:215 (1993)) by digestion with BamHI and Dral, and the fragment was isolated and directionally ligated into the pTM1(ΔNcoI) vector cleaved with BamHI and Stul. The resulting clone, pTM1(ΔNcoI)-GP, was used as the target DNA for all site-directed mutagenesis reactions. This clone encodes a GP sequence that differs from the wild-type amino acid sequence in a single residue within the membrane-spanning sequence (1662V) and, for comparative purposes, will be referred to as the wild-type sequence. This mutation is present in the original pGEM-EMGP1 clone but does not appear to affect the processing or function of the GP. GP residue numbering commences with the methionine of the signal sequence and is continuous through the GP₁ and GP₂ sequences.

Site-directed mutagenesis targeted conserved cysteines and asparagines in conserved N-linked glycosylation sites. Table 3 (below) shows 21 mutant GPs that were used for the analysis of GP₁ and GP₂ in the VV-T7 system. Additional mutants (T42D; double substitutions for the GP₁ cysteines [C108G/C135S, C108G/C147S, C121G/C135S, and C121G/C147S], C670A, C672A, C670A/C672A, and Δ309-489) were also generated but were used only in the pseudotyping experiments.

Mutagenesis of plasmid DNA sequences was performed by using commercial kits, either the GeneEditor in vitro system (Promega Corp., Madision, Wis.) or the MORPH system (5 prime→3 prime, Inc., Boulder, Colo.), according to the manufacturer's instructions. Briefly, 5′-phophorylated mutagenic primers ranging in length from 25 to 36 nucleotides (mismatches centered in the sequence) were annealed to denatured pTM1(ΔNcoI)-GP DNA and extended with T4 DNA polymerase, and the ends were ligated with T4 DNA ligase. Plasmid DNAs with mutations were enriched by specific antibiotic selection (GeneEditor, Promega, Inc., Madison, Wis.) or digestion with DpnI prior to transformation (MORPH, Promega, Inc., Madison, Wis.). Mutant DNAs were used to transform Escherichia coli mismatch repair mutants (BMH 71-18 or MORPH mutS cells), and miniprep DNA was isolated (5 Prime→3 Prime, Boulder, Colo.) and used in the second-round transformation of E. coli JM109 to isolate mutated DNA strands. The GP clone in which the mucin region was deleted (Δ309-489) was generated from two PCR clones linked by an XbaI restriction site, which resulted in the replacement of the mucin sequence with two residues (serine and arginine). Mutations in isolated plasmid clones were identified by direct sequencing of miniprep DNA with dye terminator cycle sequencing reactions (ABI) analyzed with either an ABI 373 or an ABI 377 sequencer. Large-scale preparations for each type of mutated plasmid DNA were made by using commercial kits (Promega, Madison, Wis. or 5 Prime→3 Prime, Boulder, Colo.). The DNA was quantified by UV absorbance readings at 260 nm and then stored at −70° C. until needed. The coding region (BamHI/SalI fragments) from plasmid pTM1(ΔNcoI)-GP and mutated versions of this DNA were separately ligated into the BamHI/XhoI polylinker sites of vector pcDNA3 (Invitrogen, Carlsbad, Calif.) and cloned in E. coli, and plasmid DNA was isolated for use in pseudotyping studies.

TABLE 3
Mutant Ebola virus GPs expressed in the VV-T7 system

| Mutant glycoprotein | Change (Δ indicates deletion) |
|---|---|
| C53G | GP₁ Cys 1 |
| C108G | GP₁ Cys 2 |
| C121G | GP₁ Cys 3 |
| C135S | GP₁ Cys 4 |
| C147S | GP₁ Cys 5 |
| N40D | GP₁ ΔN-linked glycan 1 |
| N204D | GP₁ ΔN-linked glycan 2 |
| N238Y | GP₁ ΔN-linked glycan 4 |
| N257D | GP₁ ΔN-linked glycan 5 |
| N277D | GP₁ ΔN-linked glycan 6 |
| N296D | GP₁ ΔN-linked glycan 7 |
| C511G | GP₂ Cys 1 |
| C556S | GP₂ Cys 2 |
| C601S | GP₂ Cys 3 |
| C608G | GP₂ Cys 4 |
| C609G | GP₂ Cys 5 |
| C670F | GP₂ Cys 6 |
| C672F | GP₂ Cys 7 |
| C670F/C672F | GP₂ Cys 6 + Cys 7 |
| N563D | GP₂ ΔN-linked glycan 1 |
| N618D | GP₂ ΔN-linked glycan 2 |

VV-T7 expression of GP sequences: Plasmid pTM1(ΔNcoI)-GP, mutated versions of this clone, and the pTM1(ΔNcoI) vector (negative control) were introduced into 293 cells infected with a recombinant vaccinia virus (vTF7-3) expressing T7 RNA polymerase (Elroy-Stein et al., P.N.A.S. USA, 86:6126 (1989)). Cells were cultured in 12-well panels to 80% confluence and then infected with vTF7-3 for 1.5 hours at a multiplicity of infection of ≧10 by using a purified virus preparation diluted in growth medium. Plasmid DNA was then introduced into infected cells by transfection. Transfection was performed by incubating a mixture of 300 μl of DMEM (minus antibiotics or serum), 1.5 μg of plasmid DNA, and 9.0 μl of Transfast (Promega, Madison, Wis.) for 15 minutes at room temperature and then adding the mixture to naked monolayers of vTF7-3-infected 293 cells that had been gently washed twice with DMEM. Cells were cultured for 1 hour, and then 1 milliliter of growth medium was added. Cells were cultured for an additional 5 hours, and then the medium was replaced with 250 μl of Eagle's minimal essential medium minus cysteine (plus antibiotics and 2% dialyzed fetal bovine serum) and containing 150 μCi of [³⁵S]cysteine/ml. After 3 hours of culturing, 300 μl of growth medium was added to each well and culturing was continued for 14 hours. At that time, supernatant fluids were removed and mixed with 66 μl of 10×TNE buffer (0.1 M Tris-HCl [pH 7.4], 1.5 M NaCl, 0.02 M EDTA) containing 10% Triton X-100 (TX-100) and 10 mM phenylmethylsulfonyl fluoride. Cell monolayers were lysed by adding 1 milliliter of 1×TNE buffer containing 1% TX-100 and 1 mM phenylmethylsulfonyl fluoride to each well and incubating the mixtures at room temperature for 5 minutes. After transfer to 1.5 milliliter Eppendorf tubes, the lysates were subjected to brief centrifugation in a microcentrifuge (9,300×g) to pellet the nuclei. Supernatant fluids were then transferred to new tubes. GP molecules were immunoprecipitated from culture supernatant fluids and cell lysates by the addition of 100 μl of a 10% staphylococcal protein A bacterial absorbent (Boehringer Mannheim) that had been preincubated for 15 minutes (with constant mixing) with rabbit anti-Ebola virus SGP-GP serum (Ruiz-Arguello et al., J. Virol., 72:1775(1998)); immunoglobulin G from 4.0 μl of serum was bound to each 100-μl volume of bacterial absorbent. Reaction mixtures were incubated at room temperature (with constant mixing) for 1 hour, and then bacterial cells were washed by three rounds of centrifugation (9,300×g) and suspended in 1 milliliter of 1×TNE buffer containing 0.5% sodium deoxycholate and 0.5% Nonidet P-40. Pelleted cells were suspended in 50 μl of 0.125 M Tris-HCl (pH 6.8) containing 2.5% sodium dodecyl sulfate (SDS), 12.5% sucrose, and 0.01% bromophenol blue. Cell suspensions were boiled for 2 minutes and pelleted for 1 minute at 16,000×g in a microcentrifuge, and equal volumes of supernatant fluids were transferred to duplicate sets of 1.5 milliliter Eppendorf tubes. One set of fluids was reduced by adding β-mercaptoethanol to a concentration of 1% (vol/vol), whereas the other was left untreated (nonreduced). Equal amounts of proteins radioimmunoprecipitated from the medium and from the cell monolayers were separated by SDS-polyacrylamide gel electrophoresis (PAGE) in 10% gels and visualized by autoradiography.

Retrovirus pseudotyping and viral transduction assays: Pseudotyped retrovirus particles consisting of MuLV cores and the Ebola GP in their envelopes were produced by transfecting wild-type or mutated plasmid DNA into gpnlslacZ cells as previously described (Sharkey et al., J. Virol., 75:2653 (2001)). Viral transduction of β-galactosidase activity into NIH 3T3 cells was determined as previously described (Sharkey et al., J. Virol., 75:2653 (2001)). All data presented are the average of the results of at least three experiments.

Immunoblot analysis of Ebola virus GP expression, processing, and incorporation into pseudotyped retroviruses: Medium from transfected ΦNX cells (Grignani et al., Cancer Res., 58:14 (1998); Pear et al., P.N.A.S. USA, 90:8392 (1993); Swift et al., (1999) Rapid production of retroviruses for efficient gene delivery to mammalian cells using 293T cell-based systems, p. 10.17.14-10.17.29. In R. Coico (ed.), Current protocols in immunology, Suppl. 31. John Wiley & Sons, Inc., New York, N.Y.) containing recombinant retroviruses was passed through a 0.45-μm-pore-size filter and centrifuged through a 30% sucrose cushion at 25,000 rpm with a Beckman 50.2-Ti rotor in a Beckman SS-71 centrifuge. The fluid was aspirated from centrifuge tubes and discarded, and the virus pellet was suspended in 100 μl of radioimmunoprecipitation assay (RIPA) buffer (140 mM NaCl, 10 mM Tris HCl [pH 8.0], 5 mM EDTA, 1% sodium deoxycholate, 1% TX-100, 0.1% SDS). Cells were treated with lysis buffer (50 mM Tris HCl [pH 8.0], 5 mM EDTA, 150 mM NaCl, 1% TX-100), and cell lysates were centrifuged in a microcentrifuge at 16,100×g for 10 minutes. The proteins in the cell lysates and the suspended virus pellet were each precipitated with a final concentration of 4% trichloroacetic acid for 2 minutes. The precipitated proteins were centrifuged in a microcentrifuge at 16,100×g for 10 minutes. The supernatant fluid was aspirated and discarded. The pellet was suspended in an equal volume of 1 M Tris and vortexed vigorously. Proteins whose glycosylation was analyzed were treated with peptide N-glycosidase F from Chryseobacterium meningosepticum (PNGase F), which removes N-linked glycosylation, and additionally with sialidase A and endo-O-glycosidase, β(1-4)-galactosidase, and glucosaminidase (ProZyme, Inc., San Leandro, Calif.), which together remove O-linked glycosylation, by following protocols provided by the supplier. The suspended pellet was mixed with a ⅙ volume of 300 mM Tris (pH 6.8)-60% (wt/vol) lycerol-4% (wt/vol) SDS-0.0012% (wt/vol) bromophenol blue-6% (vol/vol) β-mercaptoethanol, and the mixture was boiled for 5 minutes.

Equal amounts of proteins, as determined by the Bradford assay, were separated by SDS-PAGE (8.5% acrylamide) and electrophoretically blotted onto nitrocellulose membranes. Membranes were immersed in reaction buffer (20 mM Tris, 137 mM NaCl, 0.1% Tween 20 [pH 7.6]) containing 1% bovine serum albumin and incubated overnight at 4° C. Blots were incubated in reaction buffer containing rabbit anti-Ebola virus SGP-GP serum (diluted 1:1,000) for 1 hour at room temperature, washed three times in reaction buffer, and reacted with a goat anti-rabbit serum-horseradish peroxidase conjugate (diluted 1:20,000 in reaction buffer) for 30 minutes at room temperature. Membranes were washed as described above and then treated with a commercial chemiluminescent substrate solution (Amersham Pharmacia Biotech), according to protocols provided by the manufacturer. Specific reactivity to GP was visualized by exposing treated blots to X-ray film.

The Ebola virus glycoprotein (GP) can be modified through glycosylation at numerous sites (FIG. 2). These sites were mutated to determine how eliminating modification would affect the glycoprotein and viral particles that included the mutated glycoprotein.

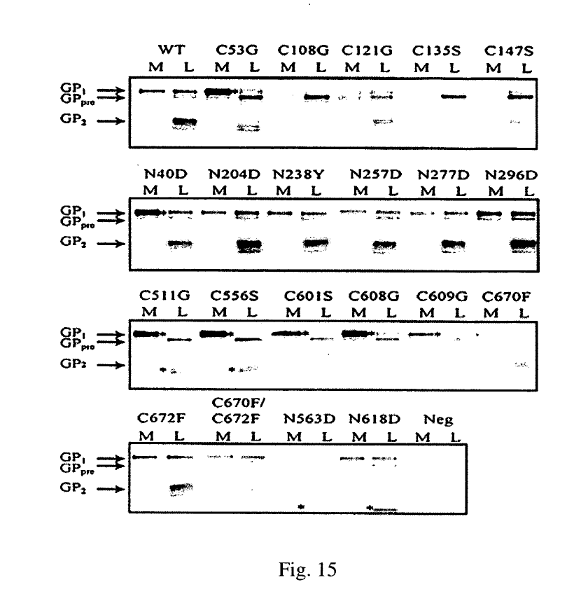

The processing of mutant Ebola virus GPs was examined through radioimmunoprecipitation assays (RIPAs) performed with glycoproteins having expression that was induced by the production of T7 RNA polymerase by a recombinant vaccinia virus (FIG. 15). The GPs listed in Table 3 were expressed in 293 cells by using a VV-T7 system and were radiolabeled with [³⁵S]cysteine. They were then immunoprecipitated with a rabbit anti-Ebola virus SGP-GP serum. The reduced proteins were analyzed by SDS-10% PAGE under reducing conditions, and autoradiography was performed. The immunoprecipitated GPs secreted or released into the medium (M) or associated with the cell monolayer (L) were run side by side. Detection of GP₂ is shown only in monolayer lanes of FIG. 15. The migration positions of GP₁, GP₂, and GPₚᵣₑ are indicated on the left. GPₚᵣₑ is an uncleaved immature or precursor form of GP that is primarily associated with the endoplasmic reticulum (ER). Asterisks in the GP₁ region identify increased levels of this GP in the medium relative to cell-associated GP₁, compared to the levels in the wild type (WT). Asterisks in the GP₂ region identify faster-migrating forms of GP₂. There are cross-reactive species migrating just slower and somewhat faster than wild-type GP₂.

In cell lysates, the wild-type protein was predominantly found in the form in which it is processed to GP₁ and GP₂, although some precursor protein that is not proteolytically processed (GPₚᵣₑ) is detected. GP₁ molecules are shed into the medium at a level equal to that of cell-associated GP₁. Mutation of the first cysteine in GP₁ (Cys-53) resulted in the secretion of most of GP₁ into the medium and a higher electrophoretic mobility for GP₂ (FIG. 15, first autoradiograph). Mutation of each of the remaining cysteines in GP₁ reduced the levels of expression of GP₁ and GP₂, and the predominant form was the GPₚᵣₑ molecule. Mutation of the second or fourth cysteine (Cys-108 and Cys-135) resulted in little or no GP₁ or GP₂ production, whereas plasmids with changes in the third or fifth cysteine (Cys-121 and Cys-147) generated small amounts of mature GP₁ and GP₂. Mutations in most of the conserved N-linked sites in GP₁ produced few changes in expression levels and patterns (FIG. 15, second autoradiograph). Elimination of the most amino-terminal site through substitution of an aspartate residue for Asn-40 caused more GP₁ to be secreted into the medium than was associated with cells. These changes to the N-linked sites in GP₁ caused no apparent changes in the migration of GP₁ or GP₂.

Mutation of any of the first through the fifth cysteines of GP₂ led to markedly increased levels of GP₁ in the medium compared to those in cell lysates. GPₚᵣₑ predominated in cell lysates, and little or no normally processed GP₂ was produced (FIG. 15, third autoradiograph). Only the C511G and C556S GP₂ molecules were easily detected, and they displayed a higher electrophoretic mobility that was similar to that for GP₂ resulting from the GP₁ C53G substitution. Substitution of the Cys-672 residue in the GP₂ membrane-spanning region with phenylalanine resulted in only a slight diminution in the expression of GP₁ and GP₂, whereas substitution of the nearby Cys-670 residue or both Cys-670 and Cys-672 with phenylalanine produced more marked reductions in expression (FIG. 15, fourth autoradiograph).

The effect of mutation of each of the two conserved N-linked glycan sites of GP₂ depended on the site that was eliminated. When the first site was changed (Asn-563), little GP₁, GP₂, or GPₚᵣₑ was detected in the medium or cell lysates (FIG. 15, fourth autoradiograph). Mutation of the second site (Asn-618) appeared to cause only a small reduction in expression, but the migration of GP₂ appeared significantly faster, presumably due to the loss in mass normally contributed by glycosylation at the site.

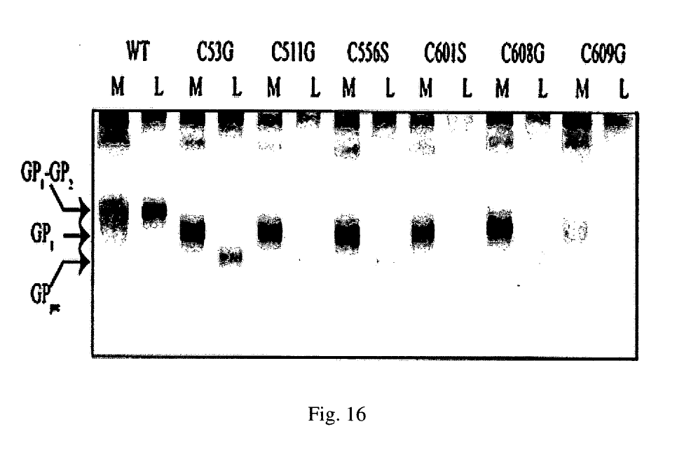

To determine whether there was any disulfide bonding between GP₁ and GP₂ for GPs in which mutated cysteines led to increased GP₁ release into the culture medium, SDS-PAGE analysis of nonreduced GP preparations was performed (FIG. 16). Whereas the mobilities of wild-type GP₁ and mutant GP₁ were identical under reducing conditions (FIG. 15), under nonreducing conditions, only wild-type GP₁ had a lower mobility that was consistent with the formation of a GP₁-GP₂ covalent heterodimer. These data suggest that the mutations affect disulfide bonding between GP₁ and GP₂ and that the most N-terminal cysteine residue of GP₁ forms the cysteine bridge with GP₂.

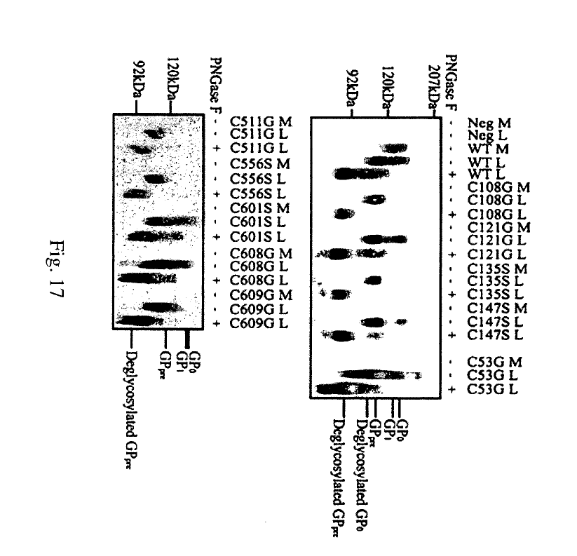

Pseudotyped retroviruses bearing GPs containing substitutions for conserved cysteines: The association of mutant GPs with recombinant retroviruses that were concentrated by ultracentrifugation and with gene transduction by the pseudotyped viruses were assayed to determine the functional consequences of the GP mutations (FIG. 17 and Table 4). ΦNX cells were transfected with plasmids encoding Ebola virus GPs. The cell lysates and viral particles collected from the culture medium were analyzed by SDS-PAGE (8.5% acrylamide) and immunoblotting with anti-Ebola virus SGP-GP antibody. An aliquot of a cell lysate was treated with PNGase F (+), which removes N-linked glycosylation was also analyzed by SDS-PAGE.

Mutation of Cys-53, which was identified as being involved in GP₁-GP₂ cysteine bridge formation, abolished the association of GP₁ with retrovirus particles as well as transduction. Viruses bearing the C108G, C121G, C135S, and C147S GPs all conveyed lower levels of transduction than did the virus bearing the wild-type GP. Each of these four mutant GPs exhibited decreased processing and association with virus particles. Two GP₁ cysteines were also simultaneously substituted. Remarkably, the virus bearing the C121G/C147S GP had only a moderately reduced capacity to transduce cells compared to the virus bearing the wild-type GP (Table 4), despite the fact that the C147S GP conferred a very low transduction capacity on pseudotyped viruses. The C108G/C135S GP possessed very modest function.

TABLE 4
Transduction of NIH 3T3 cells by virus pseudotyped with mutant
Ebola virus GPs with substitutions for cysteine residues

| | % Transduction (Relative to that for |
|---|---|
| | the wild type. The average |
| | transduction by virus bearing a wild- |
| | type GP was 1.5 × 10⁴ transducing |
| Mutant glycoprotein | units/ml) |
| C53S | <0.1% |
| C108G | 8.2 +/− 4.3% |
| C121G | 59 +/− 8% |
| C135S | <0.1% |
| C147S | 2.3 +/− 2.0% |
| C108G/C135S | 1.1 +/− 1.0% |
| C108G/C147S | <0.1% |
| C121G/C135S | 0.5 +/− 0.5% |
| C121G/C147S | 72 +/− 30% |
| C511G | <0.1% |
| C556S | <0.1% |
| C601S | <0.1% |
| C608S | <0.1% |
| C609S | <0.1% |
| C670F | 57 +/− 7% |
| C672F | 76 +/− 4.0% |
| C670F/C672F | <0.1% |
| C670A | 113 +/− 21% |
| C672A | 98 +/− 28% |
| C670A/C672A | 82 +/− 15% |

Mutation of each of the ectodomain cysteines in GP₂ (Cys-511, Cys-556, Cys-601, Cys-608, and Cys-609) resulted in a reduction in the ratio of cell-associated mature GP₁ to GPₚᵣₑ, a minimal association of GP₁ with retrovirus particles (FIG. 17), and the complete abolition of transduction (Table 4). Similar levels of the processed forms of the C601S and C608G GPs were detected and these levels were higher than those of the processed forms of the C511G, C556S, and C609G glycoproteins.

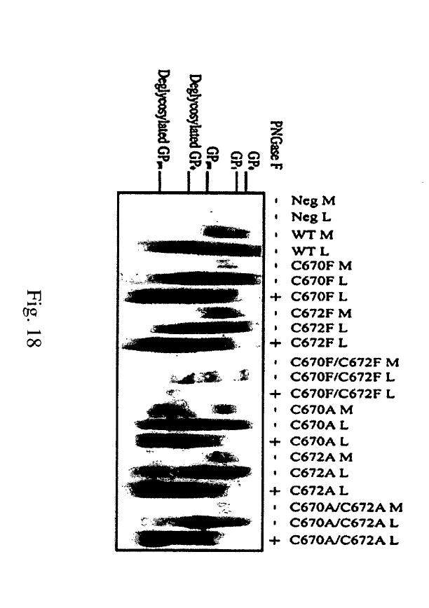

The two cysteines within the membrane-spanning sequence of GP₂ (Cys-670 and Cys-672) are palmitoylated (Ito et al., J. Virol., 75:1576 (2001)). Substitution of either Cys-670 or Cys-672 or both with alanine residues did not have major effects on GP processing and association with retrovirus particles (FIG. 18) when assayed as described with reference to FIG. 17, nor did it affect function in the transduction assay (Table 4). Substitution of Cys-670 with phenylalanine decreased transduction by 43% and greatly reduced, but did not eliminate, GP processing and association with virus particles. The C672F mutation led to a 24% decrease in transduction, and the GP was processed and incorporated into virus particles at nearly wild-type levels. The double mutant C670F/C672F was expressed and associated with virus particles at greatly diminished levels and showed a complete loss of transduction capacity.

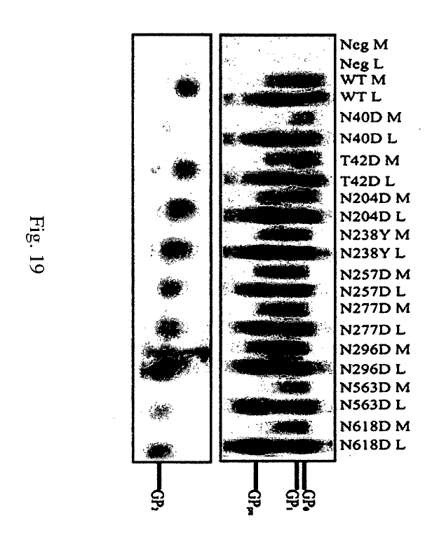

Pseudotyped retroviruses bearing GPs with altered glycosylation: The effects of mutating conserved N-linked glycosylation sites (N-X-T/S) on pseudotyping and transduction were measured according to the method used to prepare FIG. 17, except that no PGNase F was used (FIG. 19 and Table 5). It was found that processing and incorporation of the mutant GPs were similar to those of the wild-type GP at six of the eight sites mutated. The mutant GP₁ molecules that had substitutions at Asn-238, Asn-257, Asn-277, and Asn-296 and that were incorporated into viruses all had increased mobilities, indicating that they had lower levels of glycosylation and therefore that these modification sites are utilized in the cell (FIG. 19). Mutation of Asn-40 (first N-linked glycan), which is near the cysteine that forms the GP₁-GP₂ cysteine bridge, and mutation of Asn-296, which is on the cusp of the variable, mucin-like region, each resulted in a reduction in viral transduction. The N40D mutation completely abolished transduction (Table 5) and greatly reduced GP processing and association with virus particles. A T42D mutation was engineered into the wild-type sequence to prevent glycan addition at Asn-40. This mutation had no effect on the transduction capacity of pseudotyped particles or on the level of expression and electrophoretic mobility of the proteins. These results suggest that the negative effects of the N40D mutation resulted not from the loss of glycosylation but rather from conformational disruption produced by the substituted residue.

TABLE 5
Transduction of NIH 3T3 cells by virus pseudotyped with
mutant Ebola virus GPs with altered glycosylation

| | % Transduction (Relative to that |
|---|---|
| | for the wild type. The average |
| | transduction by virus bearing wild- |
| | type GP was 1.4 × 10⁴ transducing |
| Mutant Glycoprotein | units/ml) |
| N40D | <0.1% |
| T42D | 113 +/− 17% |
| N204D | 102 +/− 14% |
| N238Y | 88 +/− 4% |
| N257D | 88 +/− 9% |
| N277D | 84 +/− 10% |
| N296D | 62 +/− 10% |
| N563D | 80 +/− 4% |
| N618D | 102 +/− 3% |
| Δ309-489 (deletion mutant) | 696 +/− 142% |

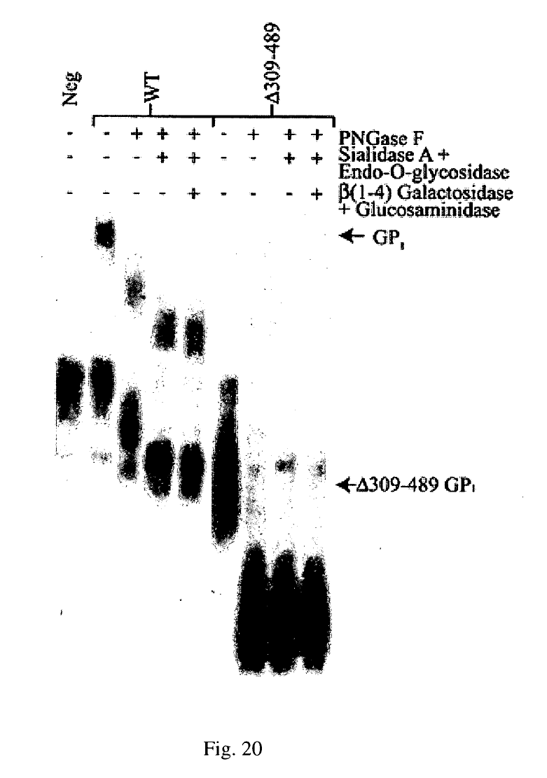

The role of O-linked glycosylation of the Ebola virus GP was examined through analysis of the effect of deletion of the region of the protein that is O-glycosylated. The processing and viral incorporation of the Δ309-489 GP were greatly enhanced (FIG. 3) when analyzed as described with reference to FIG. 4, and there was a corresponding sevenfold increase in transduction by the Δ309-489 GP-pseudotyped viruses (Table 5). The absence of an increase in the mobility of the Δ309-489 GP with sialidase A and endo-O-glycosidase treatment provides confirmation that the region of GP that is O-glycosylated was removed (FIG. 20). A glycosylated serum protein showing a mobility intermediate between those of wild-type GP₁ and Δ309-489 GP₁ was detected. The heterogeneous mobility of the PNGase F-treated proteins was indicative of the incomplete removal of N-glycosylation.

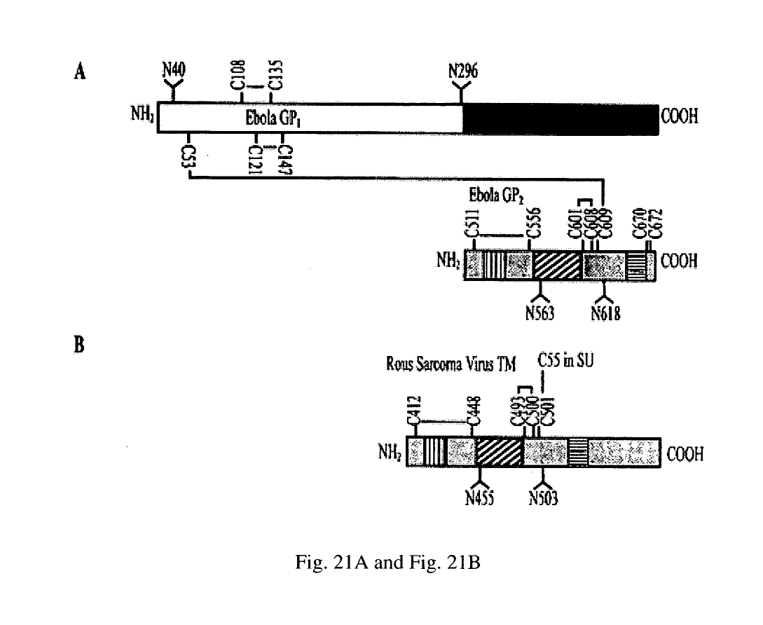

The combination of sequence analysis, structure determination, and results presented herein suggests a model for a cysteine bridge map for the Ebola virus glycoprotein (FIG. 21A). The extracellular domains of the Ebola virus GP contain ten conserved cysteine residues, five in GP₁ and five in GP₂. The five cysteines in Ebola virus GP₂ are conserved not only in Marburg virus GP₂ but also in avian sarcoma and leukosis virus (ASLV) transmembrane domain (TM), which are known to be linked by a stable disulfide bond to their envelope protein surface (SU) components (FIG. 21B). On the basis of sequence analysis and X-ray diffraction studies of Ebola virus GP₂, a putative structure for the linkage of Ebola virus GP₁ and GP₂ has taken shape. The first cysteine in GP₁ (Cys-53) had been predicted to be linked to the last cysteine in the extracellular domain of GP₂ (Cys-609) (Gallaher, Cell, 85:477 (1996); Sanchez et al., J. Virol., 72:6442 (1998)). The involvement of the first cysteine in GP₁ in the disulfide linkage to GP₂ has been confirmed herein. Substitution of a glycine for Cys-53 led to the release of most of GP₁ into the medium in the VV-T7 expression system (FIG. 15), with no evidence of C53G GP₁ being disulfide linked to GP₂ in either the medium or the cell (FIG. 16). This amino-terminal cysteine is conserved in the GPs of filoviruses, and it is thought that it also links Marburg virus GP₁ to GP₂. It is thought that the disulfide bond between GP₁ and GP₂ is reduced by a cellular enzyme on entry of the Ebola virus through analogy to the thiol-disulfide exchange reactions that take place in MuLV GPs (Pinter et al., J. Virol., 71:8073 (1997); Sanders (2000) Sulfhydryl involvement in fusion mechanisms, p. 483-514. In H. Hilderson and S. Fuller (ed.), Fusion of biological membranes and related problems. Kluwer Academic/Plenum Publishers, New York, N.Y.; Sanders, 2000. Sulfhydryl involvement in fusion mechanisms, p. 483-514. In H. Hilderson and S. Fuller (ed.), Fusion of biological membranes and related problems. Kluwer Academic/Plenum Publishers, New York, N.Y.).

The results presented herein also support the proposed linkage between Cys-601 and Cys-608 and the linkage between Cys-511 and Cys-556 (FIG. 21A). Four cysteine residues in the Ebola virus GP₁ molecule, Cys-108, Cys-121, Cys-135, and Cys-147, are likely to be linked in two intramolecular cysteine bridges (FIG. 21A). The GP containing the C121G mutation showed the least impairment of function (Table 4), suggesting that the disulfide bonding partner of Cys-121 may not be exposed on the surface (Ellgaard et al., Science, 286:1882 (1999); Sanders, 2000. Sulfhydryl involvement in fusion mechanisms, p. 483-514. In H. Hilderson and S. Fuller (ed.), Fusion of biological membranes and related problems. Kluwer Academic/Plenum Publishers, New York, N.Y.).

### EXAMPLE IV

### Use of Pseudotyped Viral Particles to Transduce Airway Epithelia

Culture of human airway epithelia: Airway epithelia were isolated from trachea or bronchi and were grown at the air-liquid interface as described (Karp et al., Methods Mol. Biol., 188:115 (2002)). All preparations used were well differentiated (>2 weeks old; resistance>1,000 Ω-cm²). A549 and H441 cell lines are derived from human lung carcinomas, and IB3 and HBE cell lines are transformed human airway cells. The cell lines HT1080 (ATCC 12012), HOS (ATCC CRL-1543), IB3 (Zeitlin et al., Am. J. Respir. Cell Mol. Biol., 4:313 (1991)), and KB (ATCC CCL-17) were maintained in Dulbecco's modified Eagle's medium (Gibco)-10% fetal bovine serum (FBS). A549 (ATCC CCL-185) cells were maintained in Dulbecco's modified Eagle's medium F12 (catalog no. 11320-033; Gibco)-10% FBS. H441 (ATCC HTB-174) cells were maintained in RPMI medium (Gibco)-10% FBS. HBE (Cozens et al., Am. J. Respir. Cell Mol. Biol., 10:38 (1994)) cells were maintained in modified Eagle's medium (Gibco)-10% FBS. In addition, each medium was supplemented with penicillin (100 U/milliliter) and streptomycin (100 μg/milliliter). In the FRα-blocking studies, the cells were washed and maintained 72 hours in RPMI medium lacking folic acid (Gibco; 27016-021) and 5% FBS prior to the addition of the blocking reagent.

Vector production: The second-generation FIV vector system utilized was previously described (Johnston et al., J. Virol., 73:4991 (1999); Wang et al., J. Clin. Investig., 104:R55 (1999)). The FIV vector construct expressed the β-galactosidase cDNA directed by the cytomegalovirus (CMV) promoter. All of the envelope constructs utilized the cytomegalovirus early gene promoter to direct transcription. Those envelopes include the vesicular stomatitis virus G protein (VSV-G), the Ebola (EBO) (Zaire strain) envelope glycoprotein (pEZGP [Jeffers et al., J. Virol., 76:12463 (2002)]), and the Marburg virus (MRB) (Musoke variant) envelope glycoprotein (pMBGP [Will et al., J. Virol., 67:1203 (1993)]). EBOΔO (pEZGP Δ309-489) has been previously described (Jeffers et al., J. Virol., 76:12463 (2002)). All of the filoviral envelope constructs were expressed from pcDNA3.1-derived plasmids (Invitrogen, Carlsbad, Calif.). Pseudotyped FIV vector particles were generated by transient transfection of plasmid DNA into 293T cells as described previously (Johnston et al., J. Virol., 73:4991 (1999)). FIV vector preparations were titered on HT1080 cells at limiting dilutions, and these titers were used to calculate the multiplicities of infection (MOIs). In addition, it was found that the filoviral glycoprotein conferred enough stability to the lentiviral vector to withstand centrifuge concentration of greater than 1,000-fold. However, the vector was typically concentrated 250-fold by centrifugation for in vitro experiments.

RNase protection assay (RPA): Folate receptor alpha (FRα) mRNA levels were determined by RNase protection assay as previously described (Sinn et al., Am. J. Physiol., 277:F634 (1999)). The FRα probe was a partial cDNA sequence cloned into pCR2.1-TOPO (Invitrogen). The human β-actin cDNA templates were obtained from Ambion (Austin, Tex.). The full-length probe for FRα and human actin were 541 and 315 base pairs, respectively. The expected protected fragment sizes were 413 and 245 base pairs, respectively. The RPA reaction was conducted by using an RPA III kit with the manufacturer's protocol (Ambion, Austin, Tex.) and was quantified with a Molecular Dynamics Storm 620 PhosphorImager System (Sunnyvale, Calif.) and the ImageQuant software provided by the manufacturer.

Fluorescence-activated cell sorter analysis (FACS): For FACS analysis, approximately 10⁶ cells were first incubated in suspension with FcγIIαCD32-blocking antibody (14531; Stem Cell Technologies, Vancouver, BC, Canada) on ice for 15 minutes. Then a monoclonal antibody against FRα (MOv18; a kind gift from Silvana Canevari [Miotti et al., Int. J. Cancer, 39:297 (1987)]) or the appropriate immunoglobulin G1 (IgG1) isotype control (554121; Pharmingen, San Diego, Calif.) was added and incubated on ice for 30 minutes. Cells were washed three times with 3% FBS in 1× phosphate-buffered saline (PBS). A goat anti-mouse fluorescein isothiocyanate (FITC)-conjugated secondary antibody (31569; Pierce, Rockford, Ill.) was then added and again incubated on ice for 30 minutes. Finally, cells were washed as before and resuspended in 500 μl of 3% FBS in 1×PBS. Data were collected by using a FACScan flow cytometer (Becton Dickinson, Franklin Lakes, N.J.), and the data were analyzed using CellQuest software.

Western blot analysis: Western blot analysis for verifying FRα protein expression was conducted by using standard techniques. Briefly, cell lysates were denatured for 5 minutes at 100° C. in Laemmli sample buffer, electrophoresed on 10% polyacrylamide gels (161-1155; Bio-Rad, Hercules, Calif.) at 125 V, and transferred to pure nitrocellulose (162-0145; Bio-Rad, Hercules, Calif.) overnight at 200 mA. The membrane was probed with a monoclonal anti-human FRα primary antibody (MOv18; 3 milligram/milliliter) at 1:1,000 and was detected by using goat anti-mouse IgG conjugated to alkaline phosphatase at a 1:1,000 dilution (A-1682; Sigma, St. Louis, Mo.).

Immunohistochemistry and confocal microscopy: Epithelial cells were rinsed with 1×PBS, fixed in 2% paraformaldehyde for 5 to 10 minutes, and rinsed with 1×PBS. The epithelial cells were then incubated for 30 minutes at 37° C. with a monoclonal anti-human FRα antibody (MOv18) or the appropriate isotype control diluted 1:100 in Hank's buffer (Gibco, Gaithersburg, Md.). The cells were washed with 1×PBS and were incubated with an FITC-conjugated anti-mouse secondary antibody (F-4143; Sigma, St. Louis, Mo.) diluted 1:100 in 1×PBS for 30 minutes at 37° C. The primary and secondary antibodies were always applied to both the apical and basolateral surfaces of nonpermeabilized cells. Images were captured with a Bio-RadMRC-1024 Hercules laser scanning confocal microscope equipped with a Kr/Ar laser (Bio-Rad, Hercules, Calif.).

Viral vector administration: Pseudotyped FIV vector was applied directly to immortalized cell lines for 4 hours at 37° C. Following incubation with the vector, cells were rinsed in media and cultured for 4 days. Following the 4-day incubation, cells were harvested and β-galactosidase activity was quantified. Primary cultures of human airway epithelial cells were transduced with pseudotyped FIV vector by diluting vector preparations in media to achieve the desired multiplicity of infection, and 100 [α]of the solution was applied to the apical surface of airway epithelial cells. After incubation for 4 hours at 37° C., the virus was removed and cells were further incubated at 37° C. for 4 days. To infect airway epithelia with pseudotyped FIV vector from the basolateral side, the Millicell culture insert containing the airway epithelia was turned over and the virus was applied to the basolateral surface for 4 hours in 100 μl of media. Following the 4 hour infection, the virus was removed and the culture insert was turned upright and allowed to incubate at 37° C., 5% CO₂, for 4 days.

β-Galactosidase quantification and AZT administration: The Galacto-light chemiluminescent reporter assay (Tropix, Bedford, Mass.) was used to quantify β-galactosidase activity following the manufacturer's protocol. The relative light units were quantified with a luminometer (Monolight 3010; Pharmingen, San Diego, Calif.) and were standardized to total protein as determined by modified Lowry assay (23240; Pierce Biotechnology, Rockford, Ill.) by using the manufacturer's protocol. To verify that the β-galactosidase activity observed in the transduced cells was due to reverse transcription-dependent expression and not the result of pseudotransduction of β-galactosidase present in the vector preparations, cells were infected in the presence or absence of zidovudine (AZT). The cells were incubated with 50 μM AZT (GlaxoWellcome, Philadelphia, Pa.) for 24 hours prior to infection and were maintained in the media following vector administration.

Administration of FRα blockers: To cleave GPI-linked cell surface proteins, cells were pretreated with 2 units of phosphatidylinositol-specific phospholipase C (PI-PLC) (P-6466; Molecular Probes, Eugene, Oreg.)/milliliter for 2 hours at 37° C. Following enzyme treatment, viral vector challenge and β-galactosidase detection proceeded as described above. To specifically block FRα, cells were preincubated with a mouse monoclonal FRα-blocking antibody (IgG1) (HFBP 458; a generous gift of Wilbur Franklin [Franklin et al., Int. J. Cancer Suppl., 8:89 (1994)]) or an isotype control antibody (554121; Pharmingen, San Diego, Calif.) for 15 minutes at room temperature. The purified blocking antibody or isotype control antibody was diluted 1:100 in Ultroser G (2-μg/ml final concentration) (Ciphergen Biosystems, Inc., Fremont, Calif.). Following antibody treatment, viral vector challenge and β-galactosidase detection proceeded as described above.

Statistics: Unless otherwise noted, all numerical data are presented as the mean plus or minus standard deviation. Statistical analysis was performed with a two-tailed, unpaired Student t test by using Microsoft Excel software.

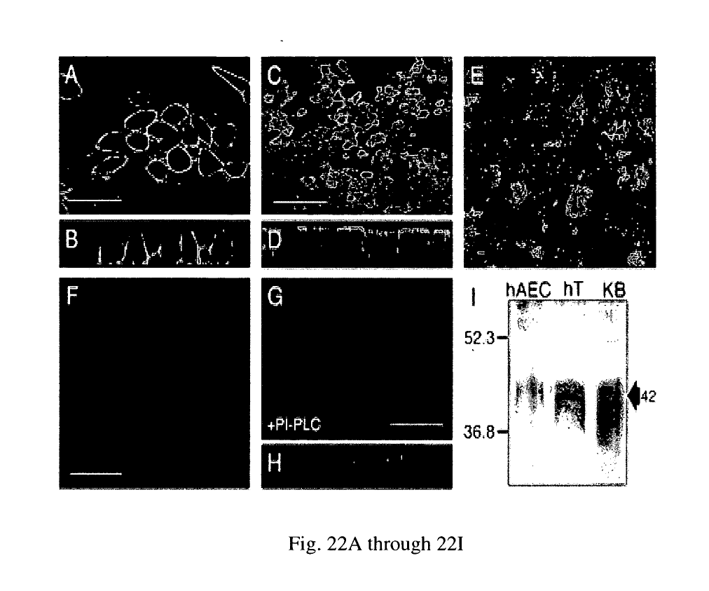

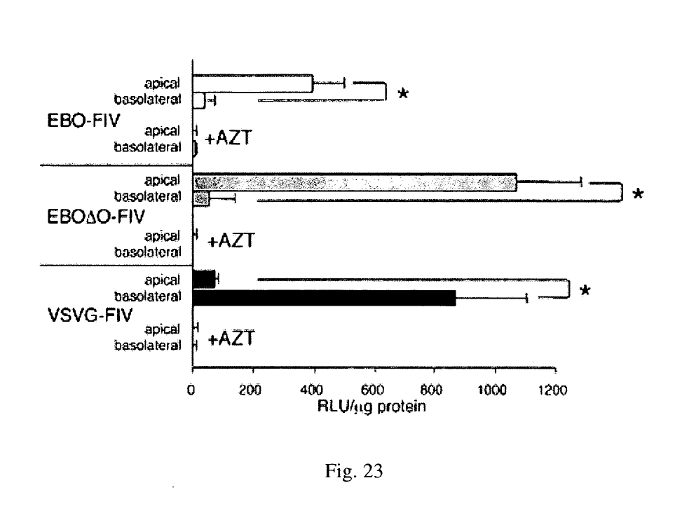

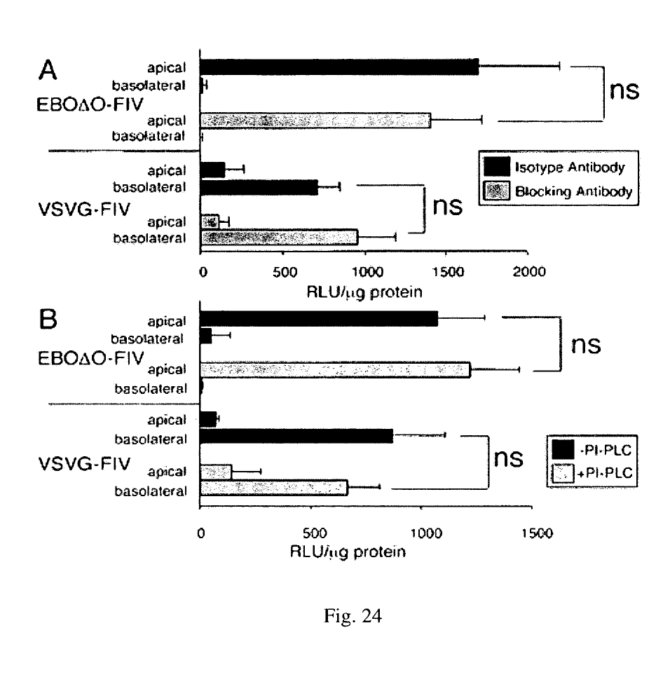

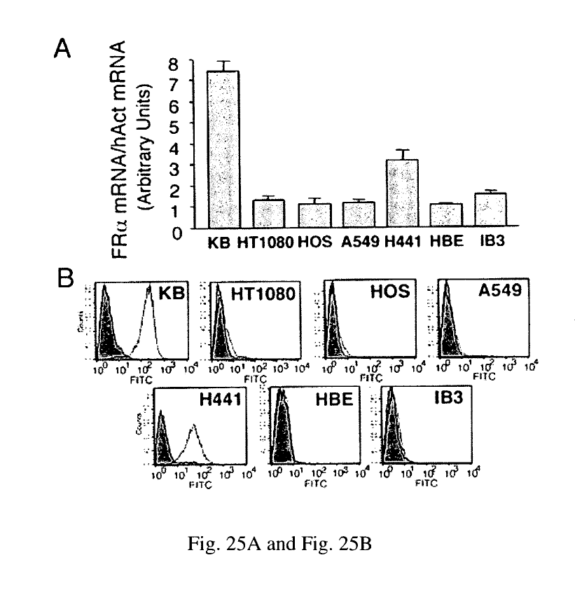

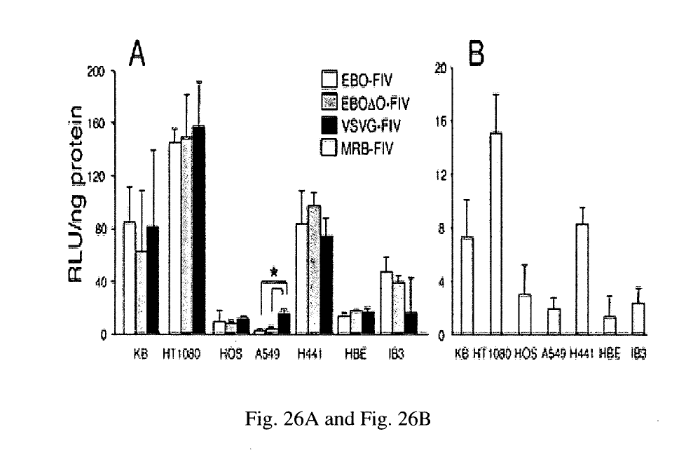

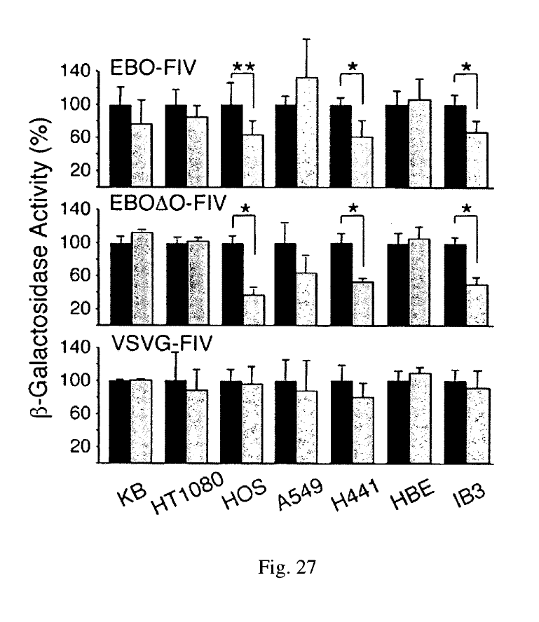

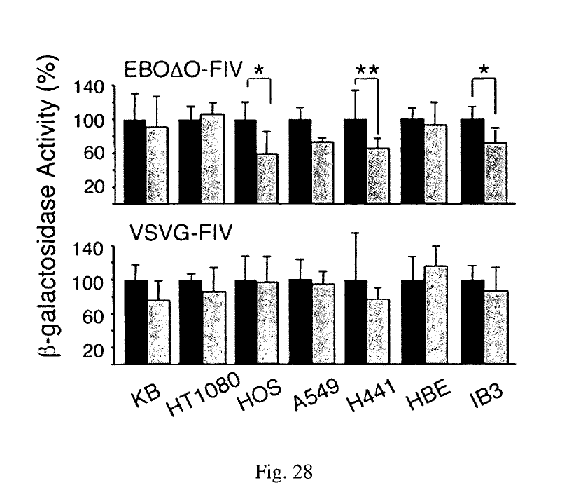

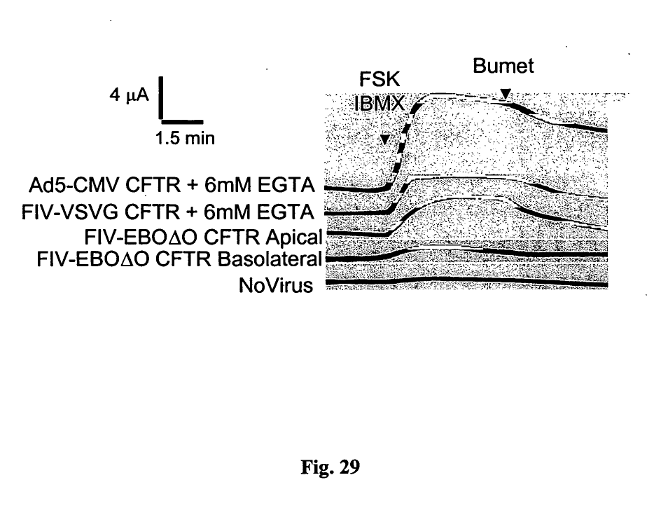

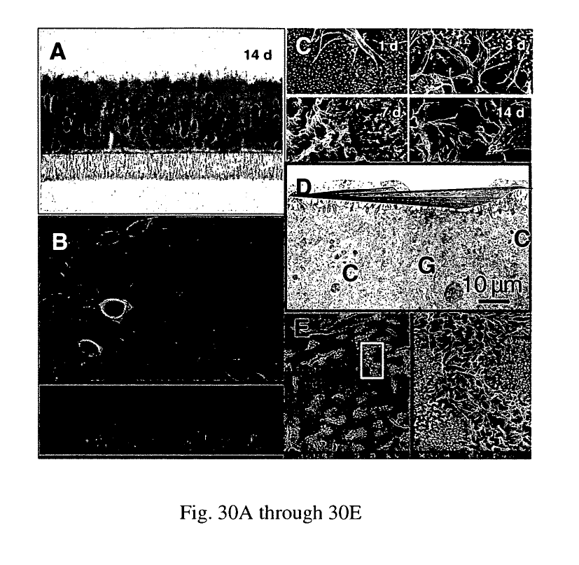

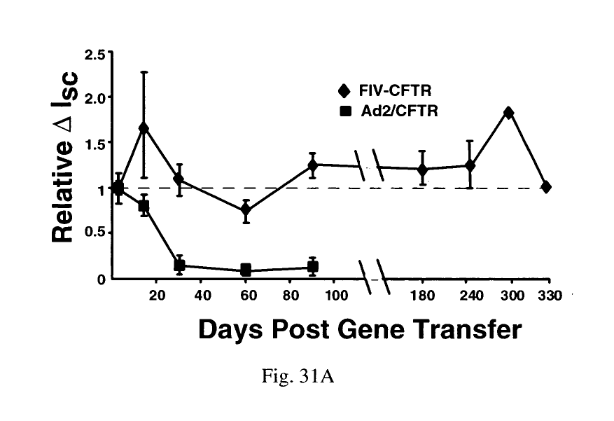

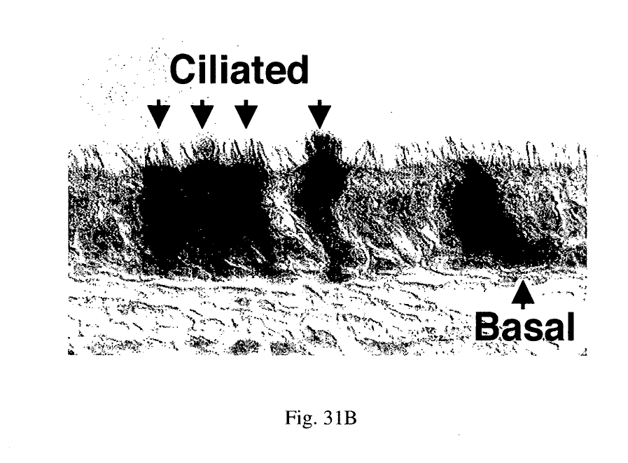

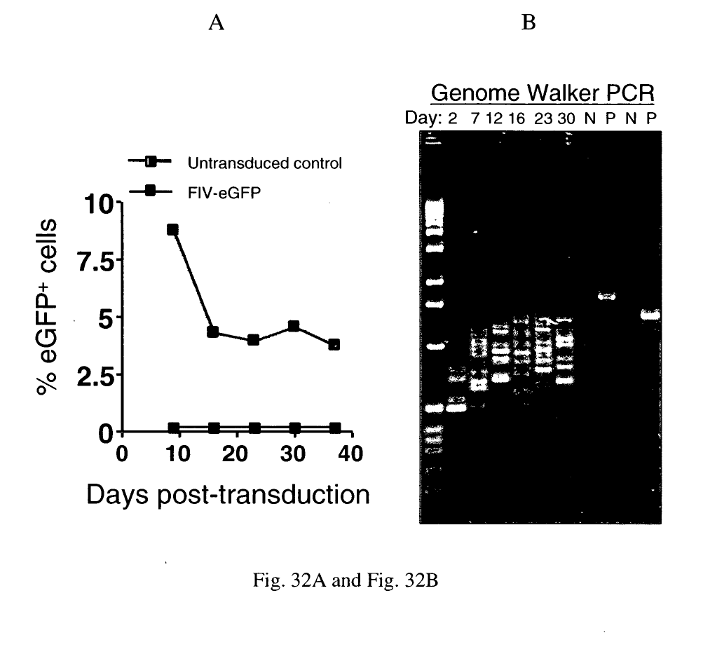

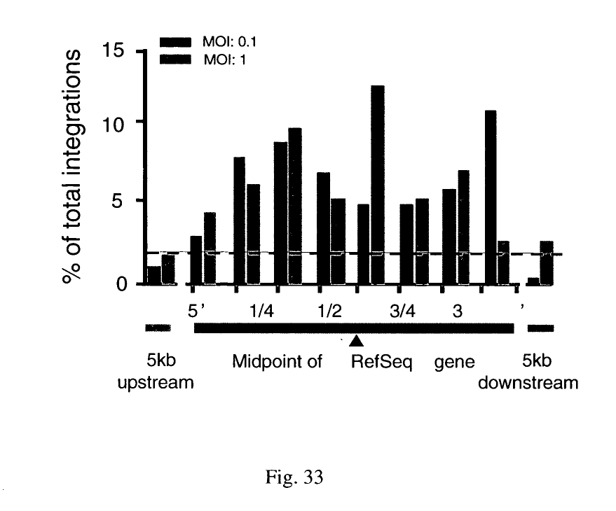

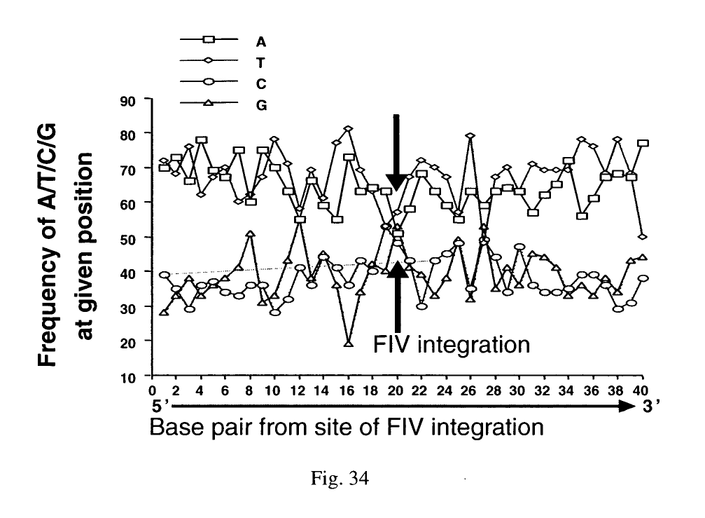

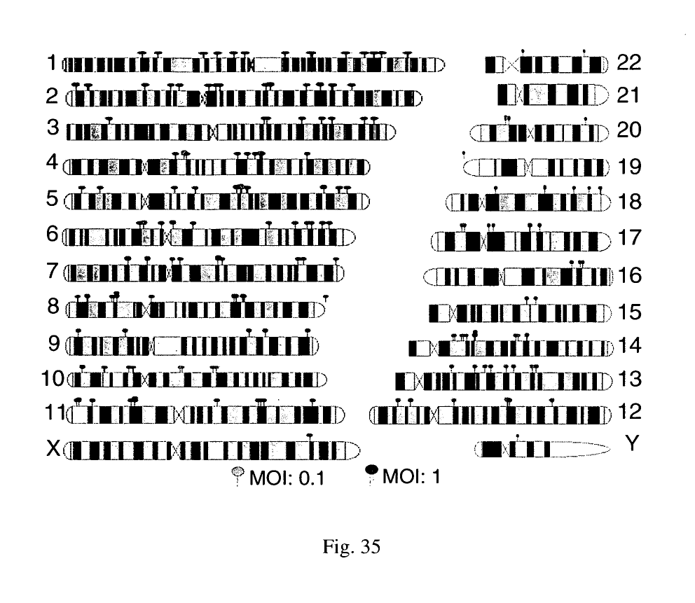

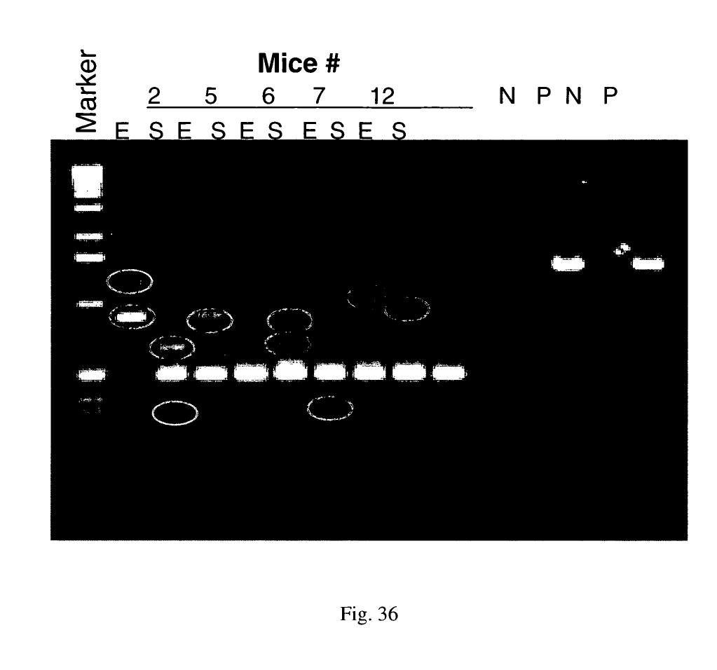

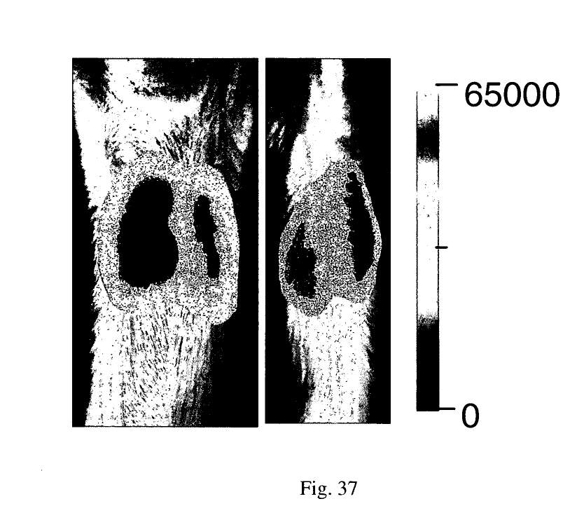

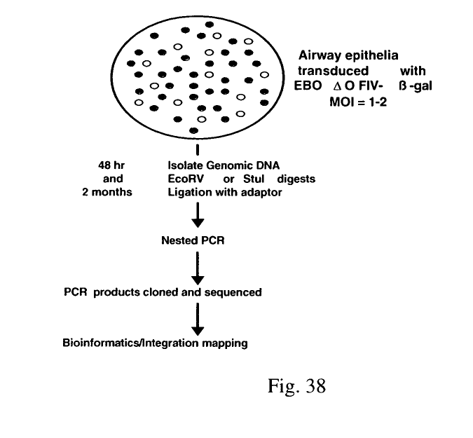

Expression of FRα in primary cultures of human airway epithelial cells: The identification of FRα as a mediator of filovirus cell entry offers the ability to investigate virus-host cell receptor interactions and pathways of infection. It has been observed that PI-PLC and FRα antiserum inhibited entry of retrovirus pseudotyped with filoviral glycoproteins in a select group of cell types, however, it was acknowledged that FRα may not facilitate virus entry into all cell types (Chan et al., Cell, 106:117 (2001)). Expression of FRα was determined in primary cultures of well-differentiated human airway epithelia. To determine the polarity of expression, primary cultures were fixed and incubated with an FRα-specific monoclonal antibody, followed by addition of an anti-mouse FITC-conjugated secondary antibody and imaged with confocal microscopy. KB, a cell line known to express FRα at high levels, exhibited abundant cell surface levels of FRα (FIG. 22A) with no polarity of expression when viewed in vertical sections (FIG. 22B). Similarly, FRα protein expression was easily detected by immunostaining primary cultures of airway epithelia (FIG. 22C). When viewed in vertical sections, FRα was abundantly expressed at the apical surface (FIG. 22D). When viewed en face at a lower magnification, the distribution of FRα was heterogeneous (FIG. 22E). This suggests that the pattern is not the result of cell-type-specific expression (e.g., ciliated versus nonciliated cells). Furthermore, the distribution was not affected by culturing cells under folate-free or excess-folate conditions. No fluorescent signal was detected when an IgG1 isotype control primary antibody and the FITC-conjugated secondary antibody were used (FIG. 22F). As an added control to verify antibody specificity, the epithelia were pretreated with an enzyme that cleaves GPI linkages, i.e., PI-PLC. PI-PLC pretreatment removed detectable FRα expression as evaluated en face (FIG. 22G) or in vertical sections (FIG. 22H). Further confirmation of FRα expression was achieved by the detection of a 42-kDa band by Western blot analysis of primary airway cells, human trachea, and KB cell lysates (FIG. 221). This demonstrates that, in a polarized sheet of primary epithelia at a given time, not all cells express FRα but that within FRα-positive cells there is substantial expression at the apical surface.

Pseudotyping FIV-based vectors with filoviral glycoproteins: Filovirus receptors are localized at the apical surface of airway epithelia. Therefore, FIV vectors were pseudotyped with filoviral glycoproteins to confer apical transduction properties to the viral particles. High viral titers facilitate in vitro entry of viral particles into cells and are of prime concern for in vivo entry of viral particles into cells as well. Titers ranging from 10⁸ to 10⁹ transducing units (TU)/milliliter are routinely attained by pseudotyping FIV-based vectors with the MuLV amphotropic, VSV, or Ross River virus envelope glycoproteins following a 250-fold centrifuge concentration (Kang et al., J. Virol., 76:9378 (2002); Wang et al., J. Virol., 72:9818 (1998); Wang et al., Methods Enzymol., 346:500 (2002); Wang et al., J. Clin. Investig., 104:R55 (1999)). However, pseudotyping FIV-based vectors with filoviral envelope glycoproteins resulted in significantly lower viral titers. As shown in Table 6, when FIV was pseudotyped with the wild-type Ebola (EBO) and Marburg (MRB) glycoproteins, average titers of 5.5×10⁶ TU/ml and 2.5×10⁴ TU/ml, respectively, were attained following a 250-fold centrifuge concentration. Therefore, alterations in the envelope constructs were designed to enhance filoviral glycoprotein incorporation into FIV virions and tested to determine their effects on viral titer.

TABLE 6
Modifications of the MRB and EBO envelope glycoproteins and
the resultant titers of pseudotyped FIV vectors^{a}

| Construct | | Increase | Mean +/− standard | |
|---|---|---|---|---|
| Name | Description of Mutation | (n-fold) | error | n |
| EBO WT | | | 5.5 × 10⁶ ± 3 × 10⁶ | 8 |
| EBOΔO | Deletion of EBO amino acids | 73.98 | 4.1 × 10⁸ ± 1 × 10⁸ | 10 |
| | 309-489 inclusive | | | |
| MRB WT | | | 2.5 × 10⁴ ± 8 × 10³ | 6 |
| MRBΔO | Deletion of MRB amino acids | 0.01 | 2.5 × 10² ± 2 × 10² | 4 |
| | 294-424 inclusive | | | |
| MRB C671A | Cysteine-to-alanine mutation at | 2.44 | 6.0 × 10⁴ ± 2 × 10⁴ | 7 |
| | position 671 | | | |
| MRB C671S | C671S Cysteine-to-serine | 0.58 | 1.4 × 10⁴ ± 9 × 10³ | 6 |
| | mutation at position 671 | | | |
| MRB C673A | C673A Cysteine-to-alanine | 1.18 | 2.9 × 10⁴ ± 1 × 10⁴ | 4 |
| | mutation at position 673 | | | |
| MRB C673S | C673S Cysteine-to-serine | 0.16 | 3.8 × 10³ ± 1 × 10³ | 4 |
| | mutation at position 673 | | | |
| MRB F676stop | F676stop Phenylalanine to stop | 2.43 | 6.0 × 10⁴ ± 5 × 10⁴ | 4 |
| | codon at position 676; | | | |
| | isoleucine to lysine at position 675 | | | |
| MRB Y679stop | Y679stop Tyrosine to stop | 1.00 | 2.5 × 10⁴ ± 2 × 10⁴ | 6 |
| | codon at position 679 | | | |

^{a}Construct names and the descriptions of the mutations are indicated. (WT) wild type. Titers are expressed as the mean (transducing unit (TU) per milliliter) plus or minus standard error. The increases (n-fold) were calculated by normalizing the corresponding titer of FIV vector pseudotyped with the wild-type glycoprotein to 1.

Deletion of the O-glycosylated region from the extracellular domain of filoviral glycoproteins: An initial strategy for enhancing filovirus glycoprotein-pseudotyped FIV vector titer was to delete an expansive region from the extracellular domain thought to be heavily O-glycosylated. By deletion of this region, the efficiency of envelope protein synthesis and of transport to the cell surface is enhanced (Jeffers et al., J. Virol., 76:12463 (2002); Example III). The deletion of amino acids 309 to 489 from the Ebola glycoprotein (EBOΔO) resulted in a marked 74-fold increase in titer over the average titer obtained with the wild-type EBO glycoprotein (Table 6). A comparable deletion in the extracellular domain of the MRB construct (MRBΔO) resulted in a loss of titer. Since potential differences between the EBO and MRB pseudotype transduction efficiencies were discovered, multiple additional avenues were pursued to increase MRB viral titer.

Mutating cytoplasmic tail acylation sites or generating cytoplasmic tail truncations of the MRB envelope glycoprotein: Studies have demonstrated that pseudotyping efficiency is influenced by the nature of the glycoprotein cytoplasmic domain (Christodoulopoulos and Cannon., J. Virol., 75:4129 (2001); Mammano et al., J. Virol., 71:3341 (1997); Zeilfelder and Bosch, J. Virol., 75:548 (2001)). Alterations to the MRB envelope glycoprotein cytoplasmic domain were designed that were intended to relieve steric interference or alter protein folding in such a way as to promote glycoprotein incorporation into the assembling virion. The MRB envelope glycoprotein contains two intracellular, potentially acylated cysteines that may interfere with efficient virion assembly. Each cysteine was mutated to either an alanine or a serine (Table 6). The C671A mutation resulted in a greater-than-twofold increase in viral titer. However, the other point mutations resulted in no titer enhancement. Incorporation of a serine at either position significantly decreased viral titer (Table 6). In addition to these point mutations, two C-terminal deletions of the MRB envelope glycoprotein were constructed. Deleting the terminal 3 amino acids (Y679stop) had no effect on FIV vector titer compared to that of the wild-type glycoprotein. However, deleting the terminal 6 amino acids (F676stop) resulted in a greater-than-twofold increase in viral titer (Table 6). The latter construct introduces a lysine at position 675 for proper anchoring of the glycoprotein in the plasma membrane. These data demonstrate the potential of carboxyl-terminal mutagenesis of the MRB envelope glycoprotein to boost vector titer.

Replacing the cytoplasmic tail of the MRB envelope glycoprotein with the MuLV amphotropic or FIV envelope cytoplasmic tail: Replacing the carboxyl-terminus of the MRB envelope glycoprotein with that of another glycoprotein known to efficiently incorporate into budding FIV virions is an additional strategy that was pursued to enhance the viral titers with the MRB glycoprotein. The amphotropic (ampho) envelope glycoprotein from MuLV was used for chimera construction. Both the MRB and ampho glycoproteins are type 1 transmembrane proteins that form homotrimers when expressed on the cell surface (Miller, P.N.A.S. USA, 93:11407 (1996) and Will et al., J. Virol., 67:1203 (1993)). In addition, similar strategies have proven effective for pseudotyping lentivirus vectors (Sandrin et al., Blood, 100:823 (2002); Stitz et al., Virology, 273:16 (2000)). Using biochemical analyses and sequence homologies of the MRB and ampho glycoproteins (Sanchez et al., Virus Res., 29:215 (1993)), the fusion site at MRB glycoprotein residue 670 and ampho 619 (termed MRB/ampho) was engineered. In addition, the MRB extracellular and transmembrane domains were fused to an ampho intracellular domain with a mutation in the putative endocytosis signal (termed MRB/amphoY665A) (Grange et al., J. Virol., 74:11734 (2000)) or a truncated ampho carboxyl-terminus (termed MRB/amphoΔ650/675). None of the MRB/ampho chimeric glycoproteins enhanced vector titers.

In addition to MRB/ampho chimeric glycoproteins, a parallel approach was pursued by using the native FIV envelope glycoprotein sequence to generate MRB/FIVenv chimeric glycoproteins. The MRB extracellular domain and transmembrane domain were fused to the native FIV envelope intracellular domain. Multiple fusion points ranging from amino acids 807 to 815 of the FIV envelope were chosen. However, none of the MRB/FIVenv chimeric constructs resulted in an increase of FIV vector titer.

Of the MRB glycoprotein mutations, only C671A and F676stop resulted in increased FIV vector titers (Table 6). However, these increases were modest and did not confer the titers conducive to multifaceted in vitro experiments with MOIs greater than 1.

Apical transduction of human airway epithelia by filovirus glycoprotein-pseudotyped FIV: To test the polarity of vector transduction in primary cultures of human airway epithelia, FIV pseudotyped with wild-type EBO glycoprotein (EBO-FIV), EBO envelope glycoprotein with the deletion of amino acids 309 to 489 (EBOΔO-FIV), or VSV glycoprotein (VSVG-FIV) was applied to the apical or basolateral surface as indicated for 4 hours at an MOI of about 5 (FIG. 23). In addition, as a control for pseudotransduction, cells were pretreated with AZT for 24 hours before vector application. Four days after initial vector incubation, cells were harvested and the 13-galactosidase activity was quantified and normalized to total protein as described above. Both EBO-FIV and EBOΔO-FIV transduced airway epithelia from the apical surface at greater efficiency than from the basolateral surface. In contrast, VSVG-FIV transduced the basolateral surface more efficiently than the apical surface. In each case, pretreating the epithelia with AZT abolished β-galactosidase expression, indicating that the observed β-galactosidase activity is not the result of pseudotransduction. These data indicate that filovirus glycoprotein-pseudotyped FIV vectors preferentially transduce airway epithelia from the apical surface, thus providing indirect evidence in support of FRα as a receptor for vector entry.

Blocking transduction of filovirus glycoprotein-pseudotyped FIV with FRα inhibitors: To examine the role of FRα as a receptor for filovirus glycoprotein-pseudotyped FIV, FRα-blocking or FRα-cleaving experiments were conducted on primary cultures of well-differentiated human airway epithelia (FIGS. 24A and 24B). In each condition, FRα-specific blocking antibody or an isotype control antibody was applied to both the apical and basolateral surfaces of the airway epithelia (FIG. 24A). Following the incubation with the antibody, the vector was administered to the apical or basolateral surface as indicated at an MOI of about five (FIG. 24A). The ability of the viral vector to transduce cells was quantified by β-galactosidase activity normalized to total protein four days after initial viral vector incubation. The FRα-blocking antibody had no effect on the VSVG-FIV control vector. Contrary to expectation, application of the FRα-blocking antibody had no effect on transduction efficacy when EBOΔO-FIV was applied to either the apical or basolateral surface compared to the isotype antibody (FIG. 24A). Apical transduction of EBOΔO-FIV remained significantly higher than basolateral transduction in the presence or absence of the blocking antibody. To complement the FRα-blocking antibody studies, additional experiments were pursued utilizing the GPI-linkage cleaving enzyme, PI-PLC, to remove FRα from the cell surface. The ability of PI-PLC to cleave FRα from primary cells was evaluated by immunofluorescence (FIGS. 22G and 22H). Cells were pretreated with PI-PLC, followed by incubation with EBOΔO-FIV or VSVG-FIV at an MOI of about five. Similar to the blocking antibody, PI-PLC did not reduce the transduction efficiency of EBOΔO-FIV or the VSVG-FIV control vector in primary cultures of airway epithelia (FIG. 24B). Again, apical transduction of EBOΔO-FIV remained significantly higher than basolateral transduction in the presence or absence of PI-PLC. These data indicate that FRα is not required as a receptor for EBOΔO-FIV in primary cultures of human airway epithelia.

Expression of FRα in immortalized airway epithelial cells: In light of this unexpected observation, the potential role of FRα as a mediator of filoviral entry into multiple immortalized cell lines was evaluated. The levels of FRα mRNA (FIG. 25A) and FRα protein (FIG. 25B) were quantified in control cell lines and airway epithelium-derived cell lines. KB is perhaps the most commonly utilized cell line for studying FRα in vitro and therefore, as expected, displayed abundant FRα mRNA and protein. The FIV vector-titering cell line, HT1080, exhibited minimal expression of FRα mRNA and protein (FIGS. 25A and 25B). HeLa and HOS cells displayed relatively high and low FRα levels, respectively. Of the four airway-derived immortalized cell lines tested (H441, HBE, A549, and IB3) only H441 exhibited relatively high levels of FRα by either RPA or FACS analysis.

Transduction of immortalized airway epithelia by filovirus glycoprotein-pseudotyped FIV: The indicated cell lines were transduced at an MOI of about 5 with EBO-FIV, EBOΔO-FIV, or VSVG-FIV (FIG. 26A). Due to titer limitations, the cell lines were transduced with wild-type MRB envelope-pseudotyped FIV (MRB-FIV) at an MOI of about 0.5 (FIG. 26B). As shown, HT1080 cells were consistently transduced with the greatest efficiency for all vectors (FIGS. 26A and 26B) despite expressing only low levels of FRα (FIGS. 25A and 25B). KB cells and H441 cells expressed much higher levels of FRα than HT1080 cells but transduced at about 50% the efficiency of HT1080 cells. IB3, HBE, HOS, and A549 were transduced at lower levels. EBO-FIV and EBOΔO-FIV transduced each cell line with similar efficiency, and the pattern of transduction between the cell lines was closely comparable with that of the MRB-FIV. The transduction efficiency of the VSVG-FIV was not significantly different from those of the EBO-FIV and EBOΔO-FIV except for the A549 cell line. VSVG-FIV transduced A549 cells at approximately fivefold-greater efficacy than EBO-FIV or EBOΔO-FIV.

Blocking transduction of filovirus glycoprotein-pseudotyped FIV with FRα inhibitors: The contribution of FRα in facilitating filovirus glycoprotein-pseudotyped FIV binding and entry into airway-derived and control cell lines was further tested by pretreating cells with an FRα-specific blocking antibody or an isotype control antibody (FIG. 27). The ability of the viral vector to transduce cells was again quantified by β-galactosidase activity normalized to total protein four days following initial incubation of the viral vector with the cells at an MOI of about 5. The transduction of KB and HT1080 cells was not significantly affected by pretreatment with the blocking antibody. These data suggest the existence of FRα-independent pathways for filoviral infection. Of the airway-derived cell lines, the blocking antibody successfully reduced transduction of the EBO-FIV and EBOΔO-FIV in H441 and IB3 cells but not in A549 or HBE cells. The FRα-blocking antibody successfully reduced the transduction efficiency in HOS cells as previously reported (Chan et al., Cell, 106:117 (2001)). EBO-FIV and EBOΔO-FIV were blocked to a similar extent in each cell line, suggesting that deleting the O-glycosylated region of the EBO glycoprotein does not alter its binding and fusion specificity or cell tropism.

Each cell line was also transduced with VSVG-FIV in the presence or absence of the FRα-blocking antibody (FIG. 27). No inhibitory effects on VSVG-FUV transduction were found for any cell line. In cells pretreated with AZT or lamivudine (not shown), β-galactosidase activity was dramatically reduced, indicating that the observed expression is not the result of pseudotransduction.

In a manner similar to that for the experiments in the primary cultures of airway epithelia, PI-PLC was utilized to remove FRα from the cell surface of the immortalized cell lines. The ability of PI-PLC to cleave FRα from immortalized cell lines was confirmed by FACS analysis. In each case the enzyme treatment efficiently removed FRα. Cells were pretreated with PI-PLC, followed by incubation with EBOΔO-FIV or VSVG-FIV at an MOI of about 5. Four days after initial vector incubation, cells were harvested and analyzed by β-galactosidase assay, and normalized to total protein. PI-PLC treatment did not inhibit transduction of EBOΔO-FIV in KB, HT1080, A549, or HBE cells (FIG. 28). However, PI-PLC treatment did reduce the transduction efficiency of EBOΔO-FIV in HOS, H441, and IB3 cells (FIG. 28). In no cell line was the transduction efficiency of VSVG-FIV affected by pretreatment of PI-PLC. Together with the blocking antibody studies, these data further support the existence of FRα-independent entry pathways for filovirus pseudotypes in human airway epithelia (FIGS. 24A, 24B and 27)(summarized in Table 7).

TABLE 7
Summary of FRα expression and EBOΔO transduction levels of
airway-derived and non-airway-derived cell lines⁺

| | | Detection Assay | | | | | |
|---|---|---|---|---|---|---|---|
| | | Result for | | Relative FRα level | | Blockage by: | |
| Cell | Tissue | Galacto- | | of: | | | Blocking |
| Type | Origin* | Lytes | Cell count | Protein | mRNA | PI-PLC | antibody |
| HT1080 | Fibroblast | +++ | +++ | Low | Low | No | No |
| HOS | Bone | + | NT | Low | Low | Yes | Yes |
| KB | Cervix | ++ | NT | High | High | No | No |
| H441 | Airway | ++ | + | High | High | Yes | Yes |
| HBE | Airway | + | +/− | Low | Low | No | No |
| IB3 | Airway | + | +/− | Low | Low | Yes | Yes |
| A549 | Airway | + | +/− | Low | Low | No | No |
| HAE | Airway | + | +/− | High** | Detected^{#} | No | No |

⁺Data from FIGS. 22 through 28 are summarized for convenience.
*all cell lines are human derived.
**data acquired by immunofluorescence and Western blotting.
^{#}data acquired by nonquantitative reverse transcriptase PCR.
HAE primary cultures of human airway epithelia.
Plus signs represent qualitative comparisons as follows:
+++ very abundant expression,
++ moderate expression;
+ detectable expression;
+/− expression detectable at limit of assay resolution; and
NT not tested.

### EXAMPLE V

### Transduction of Nucleic Acids into Airway Epithelial Cells

Partial correction of the cystic fibrosis defect with a filovirus pseudotyped FIV vector that expressed cystic fibrosis transmembrane conductance regulator (CFTR) cDNA: Primary air-liquid interface cultures of well differentiated CF airway epithelia were exposed to the following vectors: 1) adenovirus type 5 expressing CFTR with the CMV promoter (multiplicity of infection (MOI) about five) applied apically formulated with 6 mM EGTA to disrupt tight junctions, 2) VSV-G pseudotyped FIV expressing CFTR with the Moloney leukemia virus LTR promoter (Wang et al., J. Clin. Invest., 104:R49 (1999)) (MOI about twenty) applied with EGTA formulation, 3) EBOΔO pseudotyped FIV expressing CFTR applied apically at an MOI of 20 (No EGTA), 4) EBOΔO pseudotyped FIV expressing CFTR applied basolaterally at an MOI of 20, and 5) no treatment. Short circuit current tracings obtained four weeks after gene transfer with the lentiviral vector and three days following gene transfer with the adeno vector are shown in FIG. 29. The VSV-G pseudotype only transduced cells when applied to the basolateral cell surface or when formulated with EGTA to transiently disrupt tight junctions (Wang et al., J. Virol., 72:9818 (1998) and Wang et al., J. Clin. Invest., 104:R49 (1999)). Cyclic AMP activated Cl channel activity was detected in cells that were treated with the FIV-EBOΔO-CFTR from the apical surface.

In Vitro Models of human airway epithelia: Primary cultures of human airway epithelia were prepared from nasal polyps, trachea and bronchi by enzymatic dispersion as previously described (Karp et al., Meth. Mol. Biol., 188:115 (2002)). Cells grown in this fashion model the differentiated airway epithelium of the conducting airways (FIGS. 30A-30E). Representative epithelia from all cultures were scanned by electron microscope and the presence of tight junctions was confirmed by electrical resistance measurements. Cystic fibrotic epithelial cells manifest the same defective Cl transport in vitro and in vivo. Cells grown in this fashion can be maintained in culture for periods of up to 12 months with preservation of morphology and transport properties (FIG. 31A-31B). Similar cultures can be prepared from mouse tracheal epithelia (FIG. 30E).

In addition to the primary cultures, immortalized cell lines from non-CF and DF508 homozygote primary cultures were prepared by using telomerase (hTERT) and the human papilloma virus 16 E6 and E7 genes (Zabner et al., Am. J. Physiol. Lung Cell Mol. Physiol., 284:L844 (2003)). These cells maintain many properties of the primary cultures including transepithelial resistance and ion transport properties that mirror those of the primaries. The cells can be passaged up to thirty times without loss of these epithelial properties and can also be maintained in culture for months as a polarized epithelium.

BrdU labeling studies were performed in the human primary culture model to address whether cells accessible from the apical surface are mitotically active. It was determined that there are surface cells of both ciliated and non-ciliated phenotype with labeled nuclei, indicating the presence of a population of cells with progenitor capacity that may be transduced with the filovirus pseudotyped vector.

Mapping FIV vector integration events in airway epithelia in vitro and in vivo: FIV integration events were analyzed in vitro in FIV-transduced human HepG2 hepatoma cells using a sensitive nested PCR-based genome walking technique (Schroder et al., Cell, 110:521 (2002)). This genome walking technique involves the following steps: 1) digesting genomic DNA with the restriction enzymes EcoRV or Stul (5 units/μg DNA), 2) ligation of an adaptor, 3) two rounds of PCR using adapter-specific and FIV-specific primers, and 4) examination of products on agarose gel. The final PCR products were then cloned using a TA cloning vector and prepared for sequencing (FIGS. 32A and 32B). Thus, 869 DNA fragments representing putative FIV integration events were amplified, cloned and sequenced.

The putative integration sites were searched against the draft sequence of the human genome using BLAT (BLAST-like alignment tool (Kent, Genome Res., 12:656 (2002)). Three criteria were applied to validate the integration sites: 1) started at the junction with the FIV terminal LTR sequence, 2) matched the draft human genome sequence for >98% of the length of a good quality sequence read, and 3) yielded a unique best hit in the BLAT search ranking. If identical sequences are obtained from different clones, they were judged to represent multiple isolates of a single integration event. Using the human genome RefSeq gene database (http://www.ncbi.nlm.nih.gov/RefSeq/), it was also determined whether an integration event occurred in a gene. The distribution of the integration sites within the genome was compared to randomly selected sites to determine if there is a systematic bias or preference in the specific locations of integration. Finally, a publicly available HepG2 microarray expression set (Stanford University) was used to investigate whether FIV-targeted genes were transcriptionally active.

The 869 sequences, representing 211 distinct FIV integration sites, were analyzed (Table 8). The FIV integration preferences were compared with those of HIV and MLV vectors (Schroder et al., Cell, 110:521 (2002) and Wu et al., Science, 300:1749 (2003)). It was found that about 75% of FIV integration occurred in a RefSeq gene and intron regions were preferred over exons (Table 9). Of the 159 RefSeq genes that had FIV integration, 139 were represented on the HepG2 microarray data set. The median expression level for these 139 genes was 138 and was about 3.8-fold higher than that of all the genes on the array (the median expression level for all the genes on the array was 36). These data indicate that FIV integration favors more actively transcribed genes. Furthermore, FIV integrated across the entire length of transcriptional units, and the regions near transcription start sites were not the favored target for FIV integration. The integration sites appear to be surrounded by A/T rich regions (FIG. 33). The FIV integration sites did not coincide with the hot spots described previously for HIV integration in a human T cell line (FIG. 34) (Schroder et al., Cell, 110:521 (2002)). For example, chromosome 19 was reported as a favored site of HIV integration (Schroder et al., Cell, 110:521 (2002)). In contrast, only one integration event was found on this chromosome. Thus, although FIV integration preference is more similar to HIV than to MLV, significant differences may exist between FIV and HIV vectors. In addition, results may vary depending on the target cell type and the proliferation status of the transduced cells.

TABLE 8
Summary of sequence read, valid BLAT hits and distinct
integration events in FIV transduced HepG2 cells

| Multiplicity of | Number of | Number of | | Number | | Number |
|---|---|---|---|---|---|---|
| Infection | sequences | BLAT hits | | Distinct | | Redundant |
| 0.1 | 318 | 196 | (61.6%) | 100 | (51%) | 2-8 |
| 1 | 551 | 429 | (77.9%) | 111 | (25.9%) | 2-39 |
| Total | 869 | 625 | (71.9%) | 211 | (33.8%) | |

TABLE 9
Comparison of integration preferences for RefSeq genes between
FIV, HIV, MLV and random integration

| Number of Integrations that landed in a RefSeq gene | | | | | | |
|---|---|---|---|---|---|---|
| Multiplicity | | | | | FIV integration | |
| of Infection | FIV | HIV | MLV | Random | Intron | Exon |
| 0.1 | | | | | 59/68 | 9/68 |
| 1 | | | | | 82/91 | 9/91 |
| Total | 75.3% | 69% | 34% | 22.4% | 88.7% | 11.3% |

The same GenomeWalker™ approach was used to map FIV integration in the liver of mice receiving systemic injection of a GP64 pseudotyped FIV vector. The host genomic sequence along with the adjoining FIV terminal LTR was amplified with a nested PCR. The patterns of the PCR products from the first experiment are shown in FIGS. 35 and 36. Eight FIV integration sites were identified and the host genomic sequences at the junction with the FIV LTR are summarized in Table 10. A publicly available mouse gene expression database can be used to determine whether FIV-targeted genes are transcriptionally active. These results show the ability to map in vivo FIV integration events.

TABLE 10
FIV integration analysis in vivo

| | Genomic | | | | | | | |
|---|---|---|---|---|---|---|---|---|
| | DNA | | | | | | Homology | |
| Clone | Length | Chromosome | Orientation | Start | End | Score | (% Identity) | E-value |
| 1 | 172 | 17 | + | 64038690 | 64038861 | 860 | 100 | 2.1e−31 |
| 2 | 159 | 1 | + | 177458161 | 177458319 | 702 | 95.6 | 2.7e−24 |
| 3 | 812 | 16 | − | 19575911 | 19576640 | 3239 | 93.7 | 6.0e−139 |
| 4 | 289 | 14 | + | 52916686 | 52916824 | 388 | 77.5 | 2.7e−10 |
| 5 | 228 | 3 | − | 129910616 | 129916391 | 151 | 71.8 | 0.48 |
| 6 | 160 | 3 | + | 85823396 | 85823479 | 159 | 69.2 | 9.9 |
| 7 | 348 | 2 | − | 35155204 | 35155385 | 196 | 62.2 | 3.6 |
| 8 | 222 | 19 | − | 34016984 | 34017160 | 188 | 62.2 | 6.0 |

The same methods used to map integration sites in hepatocytes were performed in immortalized human airway epithelia (Zabner et al., Am. J. Physiol. Lung Cell Mol. Physiol., 284:L844 (2003)). Cells were transduced with VSV-G-FIV at an MOI of 2 and genomic DNA isolated 48 hours later. The DNA was digested with EcoRV or Stul and ligated to an adapter. Nested PCR was performed with adapter primers and FIV specific primers (Genomewalker™, Clontech, Palo Alto, Calif.). The products from the second round of PCR were sub-cloned, 20 clones, and sequenced. Six sequences were identified with junctions between the vector LTR and genomic DNA. These were mapped onto the human genome sequence. Of these 6 sequences, 5 mapped with high fidelity to sites of integration on five different chromosomes (Table 11). All sites of integration were within introns of known or predicted genes. All of the neighboring genes identified are represented in a cDNA library that was generated from normal and CF primary cultures of airway epithelia. The data show the feasibility of the method and suggest that FIV integration occurs within transcriptionally active genes in human airway epithelia.

TABLE 11

| Sequence | Site of Integration | Unigene |
|---|---|---|
| E2GM | chrom 20: intron 1 of KIAA0406 | Hs.158249 |
| S1GM | chrom 6: intron 30 of UTRN (utrophin) | Hs.286161 |
| S29GM | chrom 10: intron 5 of VTI1A | Hs.189575 |
| S32GB | chrom 4: intron 1 of KIAA1350 | Hs.101799 |
| S7GB | chrom 2: intron 13 of EIF2AK3 | Hs.102506 |

Enhancing in vivo gene transfer efficiency using viscoelastic gels formulations: Viscoelastic gels may be used to enhance in vivo transduction of airway epithelial cells by viral vectors by transiently inhibiting mucociliary clearance of the vector. Inhibition of clearance is thought to increase the opportunity for the vector to enter the target cell. Using an adenovirus expressing nuclear targeted β-galactosidase, mice were given 5×10⁸ infectious units of vector in 50 μl intratracheally formulated with or without a viscoelastic gels containing 1% carboxymethylcellulose or methylcellulose. Three days later the mice were sacrificed and tissues fixed and stained with X-gal to visualize gene expression. Animals receiving the vector in a gel had a dramatic increase in gene transfer efficiency with a marked pattern of conducting airway transduction with little expression in the alveolar compartment. This formulation approach provides a simple means to increase the residence time for a gene transfer vector on the cell surface. Such gels are thought to be useful with filovirus pseudotyped viral particles.

Non-invasive assays for in vivo monitoring of transgene expression: A luciferase reporter can be detected as a bioluminescent signal with a CCD camera (Contag et al., Nat. Med., 4:245 (1998) and Lipshutz et al. Mol. Ther., 3:284 (2001)). This method involved combining CT and 3-D bioluminescence pulmonary imaging in mice (NIH R21/33 EB001685). BALB/c mice received 5×10⁸ international units (IU) of adenovirus expressing luciferase by nasal instillation. The animals were given luciferin intraperitoneally 72 hours later and imaged in a light tight box with a CCD camera. FIG. 37 shows the luminescence signal detected over the anterior chest. It is thought that this approach can be used to monitor transgene expression longitudinally in cohorts of animals. In addition, the approach may be used with FIV pseudotyped with Ebola GPs.

Identification of cell types that express β-galactosidase or eGFP transgenes: Standard morphometry and histochemistry may be used with light microscopy or confocal microscopy to identify cell types that express β-galactosidase or eGFP transgenes. Labeled cells can be identified by their physical characteristics: 1) ciliated cells are columnar cells with cilia, 2) goblet cells are columnar cells containing secretory granules, 3) basal cells are basally located cuboidal cells having no contact with the mucosal surface, and 4) intermediate cells are columnar cells in the lower half of the epithelium having no contact with the lumen. In specific cases it may be necessary to identify cells with histochemical markers. Surface cells will be characterized as ciliated (keratan sulfate+, cytokeratin 18+), nonciliated, or goblet (PAS+). Basal cells are characterized by a basolateral cell membrane in contact with the basal lamina and positive immunostaining for cytokeratin 14. Intermediate cells are also CK14 positive.

A method to identify a receptor on airway epithelia cells that is recognized by a filovirus glycoprotein: FIV vectors pseudotyped with EBOΔO will be applied in limiting dilutions to 43 cell lines (Di Pasquale et al., Nat. Med., 9:1306 (2003)). The virus titer on each cell line will be determined 48 hours later by X-gal staining. The transduction efficiencies will be compared to gene expression data from cDNA arrays developed for a panel of 60 human tumor cell lines (NCI60 and available from the Developmental Therapeutics program, NCI website) to identify candidate proteins as filovirus receptors. For comparison to the array data, the average of the relative transduction efficiency for all the cells will be determined and a log transformation of the deviation from the average for each cell type will be determined to generate a profile. This file will then be used as a seed for COMPARE analysis. Statistical analysis can be performed using SAS software package (SAS Institute, Carey, N.C.) (Di Pasquale et al., Nat. Med., 9:1306 (2003)). Any candidate identified by this screen can be followed up by additional experimental verification. Such studies may include complementation of deficient cell lines with candidate genes and the use of blocking antibodies or inhibitory ribonucleic acid (RNAi) (Xia et al., Nat. Biotechnol., 20:1006 (2002)) to inhibit gene transfer via a candidate receptor. Any receptor protein that is identified may also be localized to determine if it is expressed on the apical, basolateral or both membranes in polarized airway epithelia, and whether its expression is required for gene transfer with filovirus pseudotypes. Phenotypic mapping using human/hamster radiation hybrid cell lines may also be used to identify receptors (Rasko et al., P.N.A.S. USA, 97:7388 (2000)).

Determining the correlation of persistent gene expression and vector integration in vitro following FIV gene transfer: A gene will be transferred with An EBOΔO and VSV-G pseudotyped FIV vector will be used to transfer a gene to epithelial cells as described above. Integration of the viral vector into the genome of the cells will be analyzed at 5 days, and 2, 4, 6, and 12 months post infection. Genomic DNA will be isolated from epithelial cells using the Gentra Puregene DNA Isolation Kit (Mpls, MN) and used as a template for Alu PCR (Butler et al., Nat. Med., 7:631 (2001)) or for confirmatory Southern analysis. Using this approach, the number of FIV integrants/cellular genome can be quantified. The basic quantitative (TaqMan™) Alu PCR method to be used has been described (Butler et al., Nat. Med., 7:631 (2001)). Integrated provirus will be detected using PCR primers complementary to the FIV LTR and chromosomal Alu repeats. Because integration will likely occur at many locations and Alu repeats are interspersed in the genome at variable intervals (Batzer and Deininger, Nat. Rev. Genet., 3:370 (2002)), it is expected that this PCR approach will generate mixed provirus-genome products of varying size. To generate a standard curve to quantify provirus copy number, a DNA standard will be generated by transducing 293T cells with FIV vector and harvesting genomic DNA after thirty days to insure loss of extrachromosomal DNA. Assays of dilutions of genomic DNA from these cells using the Alu PCR primer set will generate a heterogeneous population of provirus-genome products. The number of integrants/genome will be quantified using a second set of primers that amplify FIV reverse transcriptase (RT) products (RT primer set) that will detect full length FIV cDNAs. The standard curve will then relate the Alu PCR signal to the number of FIV provirus cDNAs present. Using this control, a sample of genomic DNA from FIV transduced airway epithelia with an unknown number of integrants can then be assayed by Alu PCR and the signal for the number of integrated FIV cDNAs can be read from the standard curve. The data can be correlated with results from the marker vector expression analysis.

Determining if FIV pseudotyped with filovirus glycoproteins cause lasting correction of the ion transport defects in CF airway epithelia: An approach that was previously taken with MLV and FIV-based vectors (Wang et al., J. Virol., 72:9818 (1998) and Wang et al., J. Clin. Invest., 104:R49 (1999)) can be used. Briefly, FIV vector pseudotyped with an EBOΔO glycoprotein and expressing the human CFTR transgene will be applied to the apical surface of well-differentiated CF epithelia at an MOI of about fifty. As positive controls, cells will be transduced with an adenoviral vector expressing CFTR, and VSV-G pseudotyped FIV will be applied to the basolateral surface using EGTA formulation (FIGS. 31A and 31B). At intervals following gene transfer, the bioelectric properties of the cells will be evaluated according to methods known in the art and described herein. The CFTR lentivirus vector preps will be titered using real time PCR as previously reported (Johnston et al., J. Virol., 73:4991 (1999)), and by p24 assay using FIV standards available from commercial veterinary supply companies.

Cyclic AMP-stimulated chloride (Cl) secretion in cultured airway epithelia will be assayed in Ussing chambers as previously described (Wang et al., J. Virol., 72:9818 (1998) and Wang et al., J. Clin. Invest., 104:R49 (1999)). The changes in short-circuit current in response to cAMP agonists are a measure of correction. Inhibitors of chloride (Cl) secretion (bumetanide, DPC) will be used to quantitate the Cl secretory current generated in response to cAMP. The results from CF epithelia treated with EBOΔO and VSV-G pseudotyped FIV-hCFTR lentiviral constructs will be compared to those of the Ad/CFTR treated CF epithelia, and normal epithelia to determine the efficacy of correction. Changes in sodium absorption (amiloride-sensitive short circuit current) will be concurrently assayed as an additional measure of correction. Individual epithelia from the same human specimen will be studied at intervals of seven days, one month, and then every two months for a total of twelve months.

Polymerase chain reaction (PCR) amplification of genomic integration sites in human airway epithelia: Primary cultures of human airway epithelia will be used in vitro to model the human surface epithelium in vivo as shown in FIG. 38 (Karp et al., Methods Mol. Biol., 188:115 (2002)). Air-liquid interface cultures will be transduced with FIV pseudotyped with EBOΔO and expressing eGFP at an MOI of one to two. At intervals after transduction, genomic DNA will be isolated for use in PCR. The average vector genome copy number for each experimental group will be verified using quantitative PCR as described above. Forty-eight hours and two months after transduction, genomic DNA will be harvested from the transduced epithelia to begin identification of integration events using GenomeWalker PCR (Clontech, Palo Alto, Calif.) as described (Schroder et al., Cell, 110:521 (2002)). This approach will involve the following steps: 1) digesting genomic DNA with the restriction enzymes EcoRV or Stul (5 units/μg DNA), 2) ligation of an adaptor, 3) two rounds of PCR using adapter-specific and FIV-specific primers, 4) examination of products on agarose gel. The final PCR products will then be cloned using a TA cloning vector (Invitrogen, Carlsbad, Calif.) and prepared for sequencing.

The putative integration sites will be searched against the draft sequence of the human genome using BLAT (BLAST-like alignment tool (Kent, Genome Res., 12:656 (2002)). The ESTprep process will be used to process the sequences (Scheetz et al., Bioinformatics, 19:1318 (2003)). A number of criteria will be applied to validate the sequence mapping results. Sequence matches from BLAT searches may be judged real if a match to the human genome: 1) starts at the junction with the FIV terminal LTR sequence, 2) matches the draft human genome sequence for >98% of the length of a good quality sequence read, and 3) yields a unique best hit in the BLAT search ranking. If identical sequences are obtained from different cells, they will be judged to represent multiple isolates of a single integration event. Sequences with high fidelity hits by BLAT will be mapped to determine their proximity to transcription units (functional class, introns vs exons), chromosomes and chromosomal regions, repetitive elements (Alu, MIR, LINE), microsatellite DNA, and human endogenous retrovirus sequences. Mapping results from FIV and MLV vectors will be contrasted to determine whether retroviral and lentiviral vectors share similar patterns of integration events. The distribution of the integration sites within the genome will be compared to randomly selected sites to determine if there is a systematic bias or preference in the specific locations of integration. Both Ensembl (www.ensembl.org) and the UCSC human genome browser (genome.ucsc.edu) provide the capability to programmatically search their databases for most of the features to be assessed for correlated proximity.

Determining transcriptional activity of genes in regions of FIV integration: Affymetrix human gene arrays (U133A chip) were used to assess the gene expression profiles from well differentiated primary cultures of airway epithelia from 10 non-CF and 10 CF lungs. The chips will be analyzed with Affymetrix's Microarray suite version 5.0 software and a global scaling adjustment will be applied with a target intensity of 1500. The global scaling adjustment will permit comparison between arrays. The output of the GeneChip software provides a qualitative assessment: ‘increase’, ‘marginal increase’, ‘no change’, ‘decrease’, and ‘marginal decrease’ and numerical values are used to make the assessment. Absolute analysis of individual chips will also be performed to provide absolute levels of expression for each gene correlated to a confidence interval. Such analysis will identify the genes that are transcriptionally active (“expressed”) near sites of FIV integration. The about 22,000 transcripts on the array will be scored by the average difference value as defined by the Affymetrix Microarray suite v.5.0 software. The genes will be distributed into bins (about 2500 gene/bin) by relative expression levels. Genes used as integration targets will then be distributed into the same bins and, based on their expression levels, summed. The Regression and ANOVA (including the mixed effects models) will be used for this analysis (Bowtell and Sambrook, DNA Microarrays: A Molecular Cloning Manual. New York: Cold Spring Harbor Laboratory Press (2003)). Because the Regression and ANOVA are a broad class of statistical techniques, the analysis model will be fine-tuned so that it is tailored to the experimental design. The software packages GeneSpring [Silicon Genetics, Redwood City, Calif.], SAS [SAS Institute, Cary, N.C.] and R [www.r-project.org] will also be used for data analysis.

Determination of what airway epithelia cell populations are targeted by the FIV vector in vivo: The gene transfer efficiency of FIV pseudotyped with the VSV-G and EBOΔO GPs will be contrasted. The in vivo efficacy of VSV-G pseudotypes formulated with 12 mM EGTA to target basolateral receptors was previously demonstrated (Wang et al., J. Virol., 72:9818 (1998) and Wang et al., J. Clin. Invest., 104:R49 (1999)), and will be used as a standard of comparison. Eight week old C57BL/6 adult mice will be administered FIV vectors with a nuclear targeted β-galactosidase reporter by intra-nasal instillation in a 100 milliliter volume (Sinn and McCray, Direct in vivo transduction by FIV-based lentivirus vectors. Airway epithelia. In: Lentivirus Gene Engineering Protocols, edited by Federico M. Totowa, N.J.: Humana Press, (2003) and Wang et al., Methods Enzymol., 346:500 (2002)). Each experimental group will consist of 8-10 animals. With a 1,000-fold concentration of FIV vector preparations, vector titers 5×10⁸ to 10⁹ TU/ml are routinely obtained.

Previous reports have indicted that the total airway surface area from trachea to bronchioles was 27.2+/−1.7 cm² in rat lungs. The total number of airway epithelial cells were 0.05×10⁹ for rat lungs. For both humans and rats, there were 18 times more alveolar cells than bronchial epithelial cells (Mercer et al., Am. J. Respir. Cell Mol. Biol., 10:613 (1994)). Assuming an average adult mouse body weight of 23 grams, the lung weight is about 230 mg (1% body weight) (Parent, Treatise on pulmonary toxicology: Comparative biology of the normal lung.: CRC Press, Inc. (1999)). Also, it can be assumed that the mouse lung equals about 16% the mass of a rat lung. Thus, a total mouse lung cell number of about 2.3×10⁸ (estimate 10⁹ cells/gram of tissue weight). Proportionally, each mouse lung would then have about 8.2×10⁶ airway epithelia and 1.4×10⁸ alveolar epithelia. Using these estimates, if all of a single vector dose reached only airway epithelia, the MOI would be about 6 (5×10⁷ IU divided by 8.2×10⁶ airway epithelia). If a single dose is equally distributed to airway and alveolar epithelia, which is more likely, the MOI achieved is about 0.3 (5×10⁷ IU divided by 1.4×10⁸ airway epithelia+1.4×10⁸ alveolar epithelia).

The transduction efficiency of a single vector dose will be compared to that of twice daily dosing over 48 hours to increase the total dose delivered (MOI). Animals will be sacrificed three weeks post gene transfer for initial assessment of gene transfer efficiency and localization of gene expression. The procedure will be replicated in three cohorts of animals. Because the age of the animal and proliferative state of the epithelium might influence the efficiency of gene transfer, the results obtained will be compared with 8 week old animals with similar cohorts of 3 week old weanlings.

The analysis will include lung tissue β-galactosidase enzymatic activity by Galactolight assay (Stein et al., Mol. Ther., 3:850 (2001)) and X-gal staining of the tissues following by morphologic assessment of transgene localization within the tissue (Wang et al., J. Clin. Invest., 104:R49 (1999)). Serial sections from the nasal and laryngeal region (where submucosal glands primarily reside in mice), the trachea and intrapulmonary airways, and the alveolar region will be examined. The transduction efficiency will also be quantified based on the relative size of the airways tranduced as described previously (Wang et al., J. Clin. Invest., 104:R49 (1999)). Cell types expressing the β-galactosidase transgene will be characterized using morphologic criteria and co-localization with cell markers as described above. Transduction of non-ciliated surface cells of the large airways, submucosal gland cells, Clara cells or alveolar type II cells would suggest progenitor cell transduction.

Determining if in vivo gene transfer with the EBOΔO pseudotyped FIV persists in the airways of the mouse: The EBOΔO pseudotyped with a marker gene will be used to transfer the marker gene to a mouse and expression will be assayed at 2, 4, 6 and 12 months. The results obtained will be contrasted with the results obtained with the VSV-G vector. The methods for the introduction of the vector and assessment of gene transfer will be essentially the same as those described above. The proviral/genomic junctions will also be determined using methods disclosed herein (Example V). After perfusion of the pulmonary circulation to remove blood, the genomic DNA will be isolated from the animals at the indicated time points for analysis as outline for the primary cell cultures. The sequenced junctions from the 2, 4, 6 and 12 month time points will be mapped on the mouse genome. Tissues will also be analyzed histologically for indicators of changes resulting from insertional mutagenesis, such as tumor formation.

A subset of integration sites from cohorts of animals 2, 4, 6 and 12 months will be PCR amplified, visualized, and mapped following gene transfer. The LAM PCR method can be used to determine integration sites in addition to methods described herein. The LAM PCR method has been described (Woods et al., Blood, 17:17 (2002)). This method is very sensitive and designed to amplify proviral/genomic junctions from complex samples. The documented sensitivity is 1-10 integration events/100,000 cells (Woods et al., Blood, 17:17 (2002)). If the yield of PCR products from total lung DNA is low, alternative approaches may be used. These might include perfusion of the trachea, airways and alveoli with a protease solution to dissociate and recover some epithelia. Such approaches are commonly used to isolate airway or alveolar epithelia in mice (Corti et al., Am. J. Respir. Cell Mol. Biol., 14:309 (1996); Davidson et al., Am. J. Physiol. Lung Cell Mol. Physiol., 279:L766 (2000); You et al., Am. J. Physiol. Lung Cell Mol. Physiol., 283:L1315 (2002)). Because of the sensitivity of Genome Walker™ and LAM PCR, DNA from a few transduced cells will yield products. Additional methods may be used to detect the persistence of gene expression in vivo using non-invasive imaging techniques. These include vectors carrying reporter constructs such as luciferase that can be detected as bioluminescent signal using a CCD camera (Contag et al., Nat. Med., 4:245 (1998)) and PET scanning (Richard et al., Am. J. Respir. Crit. Care Med., 27:27 (2002)).

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. A method to introduce a selected nucleic acid sequence into an airway epithelial cell comprising:
contacting the airway epithelial cell with a pseudotyped retrovirus, wherein the pseudotyped retrovirus comprises a glycoprotein in which a portion of an O-glycosylation region within the glycoprotein has been deleted and a retroviral capsid comprising the selected nucleic acid sequence.

2. The method according to claim 1, wherein the glycoprotein has reduced glycosylation when compared to glycosylation of a glycoprotein in which a portion of the O-glycosylation region has not been deleted.

3. The method according to claim 1, wherein the glycoprotein is a filoviral glycoprotein.

4. The method according to claim 1, wherein the filoviral glycoprotein is a Marburg virus glycoprotein.

5. The method according to claim 3, wherein the filoviral glycoprotein is an Ebola virus glycoprotein.

6. The method according to claim 5, wherein the Ebola virus glycoprotein lacks amino acids 309-489.

7. The method according to claim 1, wherein the retroviral capsid is a lentiviral capsid.

8. The method according to claim 7, wherein the lentiviral capsid is from feline immunodeficiency virus.

9. The method according to claim 1, wherein the airway epithelial cell is a mammalian airway epithelial cell.

10. The method according to claim 1, wherein the airway epithelial cell is a human airway epithelial cell.

11. The method according to claim 1, wherein the pseudotyped retrovirus is contacted with the airway epithelial cell on an apical surface of the airway epithelia cell.

12. The method according to claim 1, wherein the pseudotyped retrovirus is contacted with the airway epithelial cell in vivo, in vitro or ex vivo.

13. The method according to claim 1, further comprising contacting the airway epithelial cell with an agent that disrupts junctions between cells.

14. The method according to claim 13, wherein the airway epithelial cell is contacted with the agent before, during or after the airway epithelial cell is contacted with the pseudotyped retrovirus.

15. The method according to claim 13, wherein the agent that disrupts junctions between cells is EGTA, EDTA or sodium citrate.

16. The method according to claim 1, wherein the selected nucleic acid sequence is present within the airway epithelial cell prior to being contacted with the pseudotyped retrovirus.

17. The method according to claim 1, wherein the selected nucleic acid sequence is not present within the airway epithelial cell prior to being contacted with the pseudotyped retrovirus.

18. The method according to claim 1, wherein the selected nucleic acid sequence encodes cystic fibrosis transmembrane regulator protein (CFTR).

19. A method to reduce or eliminate symptoms of cystic fibrosis in a mammal comprising:
contacting an airway epithelial cell of the mammal with a pseudotyped retrovirus that comprises a glycoprotein in which a portion of an O-glycosylation region within the glycoprotein has been deleted and a retroviral capsid that comprises a nucleic acid sequence that encodes a cystic fibrosis transmembrane regulator protein.

20. The method according to claim 19, wherein the glycoprotein has reduced glycosylation when compared to glycosylation of a glycoprotein in which a portion of the O-glycosylation region has not been deleted.

21. The method according to claim 19, wherein the glycoprotein is a Marburg virus glycoprotein.

22. The method according to claim 19, wherein the glycoprotein is an Ebola virus glycoprotein.

23. The method according to claim 19, wherein the Ebola virus glycoprotein lacks amino acids 309-489.

24. The method according to claim 19, wherein the retroviral capsid is a lentiviral capsid.

25. The method according to claim 24, wherein the lentiviral capsid is from feline immunodeficiency virus.

26. The method according to claim 19, wherein the mammal is a human.

27. The method according to claim 19, wherein the pseudotyped retrovirus is contacted with an apical surface of the airway epithelia cell.

28. The method according to claim 19, further comprising contacting the airway epithelial cell with an agent that disrupts junctions between cells.

29. The method according to claim 28, wherein the airway epithelial cell is contacted with the agent before, during or after the airway epithelial cell is contacted with the pseudotyped retrovirus.

30. The method according to claim 28, wherein the agent that disrupts junctions between cells is EGTA, EDTA or sodium citrate.


SEQUENCE LISTING (16 sequences)

SEQ ID NO: 1 - protein, 681 aa, Marburg virus glycoprotein
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWGVLTNLGILLLLSIAVLIALSCICRIFTKYIG

SEQ ID NO: 2 - DNA, 2046 bp, Marburg virus glycoprotein
atgaagaccacatgtctctttatcagtcttatcttaatccaagggataaaaactctccctattttagagatagctagtaacaatcaaccccaaaatgtggattcggtatgctccggaactctccagaagacagaagatgtccatctgatgggattcacactgagtgggcaaaaagttgctgattcccctttggaggcatccaagcgatgggctttcaggacaggtgtacctcccaagaatgttgagtatacagaaggggaggaagccaaaacatgctacaatataagtgtaacggatccctctggaaaatccttgctgttggatcctcctaccaacatccgtgactatcctaaatgcaaaactatccatcatattcaaggtcaaaaccctcatgcgcaagggatcgccctccatttgtggggagcatttttcctgtatgatcgcattgcctccacaacaatgtaccgaggcagagtcttcactgaagggaacatagcagctatgattgtcaataagacagtgcacaaaatgattttctcgaggcaaggacaggggtaccgtcacatgaatctgacttctactaataaatattggacaagtaacaatggaacacaaacgaatgacactggatgcttcggtgctcttcaagaatacaactccacgaagaatcaaacatgtgctccgtccaaaataccctcaccactgcccacagcccgtccagagatcaaacccacaagcaccccaactgatgccaccacactcaacaccacagacccaaacaatgatgatgaggacctcataacatccggttcagggtccggagaacaggaaccctatacaacttcagatgcggtcactaagcaagggctttcatcaacaatgccacccactccctcaccacaaccaagcacgccacagcaagaaggaaacaacacagaccattcccaaggtactgtgactgaacccaacaaaaccaacacaacggcacaaccgtccatgcccccccacaacaccactgcaatctctactaacaacacctccaagaacaacttcagcaccctctctgtatcactacaaaacaccaccaattacgacacacagagcacagccactgaaaatgaacaaaccagtgccccctcgaaaacaaccctgcctccaacaggaaatcttaccacagcaaagagcactaacaacacgaaaggccccaccacaacggcaccaaatatgacaaatgggcatttaaccagtccctcccccacccccaacccgaccacacaacatcttgtatatttcagaaagaaacgaagtatcctctggagggaaggcgacatgtttccttttctggacgggttaataaatgctccaattgattttgatccagttccaaatacaaagacgatctttgatgaatcttctagttctggtgcttcggctgaggaagatcaacatgcctcccccaatatcagtttaactttatcctattttcctaatataaatgaaaacactgcctactctggagaaaatgagaacgattgtgatgcagagttaagaatttggagcgttcaggaggatgacctggcagcagggctcagttggataccgttttttggccctggaatcgaaggactttatactgctggtttaattaaaaaccaaaacaatttggtctgcaggttgaggcgtctagccaatcaaactgccaaatccttggaactcttattaagagtcacaaccgaggaaaggacattttccttaattaatagacatgccattgactttctactcacaaggtggggaggaacatgcaaagtgcttggacctgattgttgcattggaatagaagacttgtccaggaatatttcggaacaaattgaccaaatcaaaaaagatgaacaaaaagaggggactggttggggtctaggtggtaaatggtggacatccgactggggtgttcttactaacttgggcattttgctactattatccatagctgtcttgattgctctatcctgtatttgtcgtatctttaccaaatatatcgggtaa

SEQ ID NO: 3 - protein, 676 aa, Ebola virus glycoprotein
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETRYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSLRPPITKPGPDNSTHNTPVYKLDISEATQVEQHHRRTDNASTTSDTPPATTAAGPLKAENTNTSKGTDLLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 4 - DNA, 2030 bp, Ebola virus glycoprotein
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacgaatcaattgagatcagttggactgaatctcgaagggaatggagtggcaactgacgtgccatctgcaactaaaagatggggcttcaggtccggtgtcccaccaaaagtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgatcgacttgcttccacagttatctaccgaggaacgactttcgctgaaggtgtcgtggcatttctgatactgccccaagctaagaaggacttcttcagctcacaccccttgagagagccggtcaatgcaacggaggacccgtctagtggctactattctaccacaattagatatcaggctaccggctttggaaccaatgagacagagtatttgttcgaggttgacaatttgacctacgtccaacttgaatcaagattcacaccacagtttctgctccagctgaatgagacaagatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcttcgaccccccataaccaaaccaggtccggacaacagcacccacaatacacccgtgtataaacttgacatctctgaggcaactcaagttgaacaacatcaccgcagaacagacaacgccagcacaacctccgacactccccccgccacgaccgcagccggacccctaaaagcagagaacaccaacacgagcaagggcactgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactattcctgagagccacaaccgagctacgcaccttttcaatcctcaaccgtaaggcaattgatttcttgctgcagcgatggggcggcacatgccacattttgggaccggactgctgtatcgaaccacatgattggactaagaacataacggacaaaattgatcagattattcatgattttgttgataaaacccttccggaccagggggacaatgacaattggtggacaggatggagacagtggataccggcaggtattggagttacaggcgttataattgcagttatcgctttattctgtatatgcaaatttgtcttttag

SEQ ID NO: 5 - protein, 1254 aa, Ross River Structural polyprotein
MNYIPTQTFYGRRWRPRPAFRPWQVSMQPTPTMVTPMLQAPDLQAQQMQQLISAVSALTTKQNVKAPKGQRQKKQQKPKEKKENQKKKPTQKKKQQQKPKPQAKKKKPGRRERMCMKIENDCIFEVKLDGKVTGYACLVGDKVMKPAHVKGTIDNPDLAKLTYKKSSKYDLECAQIPVHMKSDASKYTHEKPEGHYNWHHGAVQYSGGRFTIPTGAGKPGDSGRPIFDNKGRVVAIVLGGANEGARTALSVVTWTKDMVTRVTPEGTEEWSAALMMCILANTSFPCSSPPCYPCCYEKQPEQTLRMLEDNVNRPGYYELLEASMTCRNRSRHRRSVTEHFNVYKATRPYLAYCADCGDGYFCYSPVAIEKIRDEAPDGMLKIQVSAQIGLDKAGTHAHTKIRYMAGHDVQESKRDSLRVYTSAACSIHGTMGHFIVAHCPPGDYLKVSFEDADSHVKACKVQYKHDPLPVGREKFVVRPHFGVELPCTSYQLTTAPTDEEIDMHTPPDIPDRTLLSQTAGNVKITAGGRTIRYNCTCGRDNVGTTSTDKTINTCKIDQCHAAVTSHDKWQFTSPFVPRADQTARRGKVHVPFPLTNVTCRVPLARAPDVTYGKKEVTLRLHPDHPTLFSYRSLGAEPHPYEEWVDKFSERIIPVTEEGIEYQWGNNPPVRLWAQLTTEGKPHGWPHEIIQYYYGLYPAATIAAVSGASLMALLTLAATCCMLATARRKCLTPYALTPGAVVPLTLGLLCCAPRANAASFAETMAYLWDENKTLFWMEFAAPAAALALLACCIKSLICCCKPFSFLVLLSLGASAKAYEHTATIPNVVGFPYKAHIERNGFSPMTLQLEVVETSWEPTLNLEYITCEYKTVVPSPFIKCCGTSECSSKEQPDYQCKVYTGVYPFMWGGAYCFCDSENTQLSEAYVDRSDVCKHDHASAYKAHTASLKATIRISYGTINQTTEAFVNGEHAVNVGGSKFIFGPISTAWSPFDNKIVVYKDDVYNQDFPPYGSGQPGRFGDIQSRTVESKDLYANTALKLSRPSPGVVHVPYTPTPSGFKYWLKEKGSSLNTKAPFGCKIKTNPVRAMDCAVGSIPVSMDIPDSAFTRVVDAPAVTDLSCQVVVCTHSSDFGGVATLSYKTDKPGKCAVHSHSNVATLQEATVDVKEDGKVTVHFSTASASPAFKVSVCDAKTTCTAACEPPKDHIVPYGASHNNQVFPDMSGTAMTWVQRLASGLGGLALIAVVVLVLVTCITMRR

SEQ ID NO: 6 - protein, 437 aa, Ross River Virus E1
EHTATIPNVVGFPYKAHIERNGFSPMTLQLEVVETSLEPTLNLEYITCEYKTVVPSPFIKCCGTSECSSKEQPDYQCKVYTGVYPFMWGGAYCFCDSENTQLSEAYVDRSDVCKHDHASAYKAHTASLKATIRISYGTINQTTEAFVNGEHAVNVGGSKFIFGPISTAWSPFDNKIVVYKDDVYNQDFPPYGSGQPGRFGDIQSRTVESKDLYANTALKLSRPSPGVVHVPYTQTPSGFKYWLKEKGSSLNTKAPFGCKIKTNPVRAMDCAVGSIPVSMDIPDSAFTRVVDAPAVTDLSCQVVVCTHSSDFGGVATLSYKTDKPGKCAVHSHSNVATLQEATVDVKEDGKVTVHFSTASASPAFKVSVCDAKTTCTAACEPPKDHIVPYGASHNNQVFPDMSGTAMTWVQRLASGLGGLALIAVVVLVLVTCITMRR

SEQ ID NO: 7 - DNA, 1311 bp, Ross River Virus E1
gagcacacagccacaattccgaatgtggtggggttcccgtataaggctcacattgaaaggaatggcttctcgcccatgactctgcagcttgaagtggtggagacaagcttggaacccacacttaacctggagtacattacctgcgaatacaagacggtggtcccttcgccattcatcaaatgttgcggaacatcagaatgctcatccaaggagcagccagactaccaatgcaaggtgtacacgggtgtatacccattcatgtggggtggagcctactgtttctgcgactccgagaacacgcagctcagcgaggcctatgtcgacaggtcagacgtttgcaaacatgatcacgcatcggcctacaaggcacacacggcctctctaaaagcaacaatcaggatcagttatggcaccatcaaccagaccaccgaggccttcgttaatggtgaacacgcggtcaacgtgggcggaagcaagttcatctttggaccgatctcaacagcttggtcaccgttcgacaataaaattgtcgtgtataaagatgatgtctacaaccaggacttcccaccctacggatcaggccagccgggtagattcggagacattcagagcaggacagtggagagcaaagacttgtatgccaacacggccctaaaactctcaagaccatcacccggggttgtgcatgtgccatacacgcagacaccatccggatttaaatattggctgaaggagaaaggatcttcattgaatacaaaggccccttttggctgcaagataaagaccaatccagtcagagccatggattgtgcagttggcagtatacctgtgtcgatggacatacctgacagtgcattcacacgagtggtagatgccccggctgtaacagacctgagctgccaggtagtggtctgtacacactcctccgatttcggaggagttgccacattgtcttacaaaacggacaaacccggcaagtgcgctgtccactcacattccaacgtcgcaacgttgcaagaggcgacggtggatgtcaaggaggatggcaaggtcacagtgcacttttccacggcgtccgcctccccggccttcaaagtgtccgtctgtgacgcaaaaacaacgtgcacggcggcgtgcgagcctccaaaagaccacatcgtcccttatggggcgagccataacaaccaggtctttccggacatgtcaggaactgcgatgacgtgggtgcagaggctggccagtgggttaggtgggctggctctcatcgcggtggttgtgctggtcttggtaacctgcataacaatgcgtcgg

SEQ ID NO: 8 - protein, 487 aa, Ross River Virus E3-E2
MSAALMMCILANTSFPCSSPPCYPCCYEKQPEQTLRMLEDNVNRPGYYELLEASMTCRNRSRHRRSVTEHFNVYKATRPYLAYCADCGDGYFCYSPVAIEKIRDEASDGMLKIQVSAQIGLDKAGTHAHTKIRYMAGHDVQESKRDSLRVYTSAACSIHGTMGHFIVAHCPPGDYLKVSFEDADSHVKACKVQYKHDPLPVGREKFVVRPHFGVELPCTSYQLTTAPTDEEIDMHTPPDIPDRTLLSQTAGNVKITAGGRTIRYNCTCGRDNVGTTSTDKTINTCKIDQCHAAVTSHDKWQFTSPFVPRADQTARRGKVHVPFPLTNVTCRVPLARAPDVTYGKKEVTLRLHPDHPTLFSYRSLGAEPHPYEEWVDKFSERIIPVTEEGIEYQWGNNPPVRLWAQLTTEGKPHGWPHEIIQYYYGLYPAATIAAVSGASLMALLTLAATCCMLATARRKCLTPYALTPGAVVPLTLGLLCCAPRANA

SEQ ID NO: 9 - DNA, 1461 bp, Ross River Virus E3-E2
atgtctgccgcgctgatgatgtgtatccttgccaacacctctttcccctgctcatcacctccctgctacccctgctgctacgaaaaacagccagaacagacactgcggatgctggaagacaatgtgaatagaccagggtactatgagctactggaagcgtccatgacatgcagaaacagatcacgccaccgccgtagtgtaacagagcacttcaatgtgtataaggctactagaccgtacttagcgtattgcgctgactgtggggacgggtacttctgctatagcccagttgctatcgagaagatccgagatgaggcgtctgacggcatgctcaagatccaagtctccgcccaaataggtctggacaaggcaggtacccacgcccacacgaagatccgatatatggctggtcatgatgttcaggaatctaagagagattccttgagggtgtacacgtccgcagcgtgctctatacatgggacgatgggacacttcatcgtcgcacattgtccgccaggcgactacctcaaggtttcgttcgaggacgcagattcacacgtgaaggcatgtaaggtccaatacaagcacgacccattgccggtgggtagagagaagttcgtggttagaccccactttggcgtagagctgccatgcacctcataccagctgacaacagctcccaccgacgaggagatcgacatgcacacaccgccagatataccggatcgcaccctgctatcacagacggcgggcaacgtcaaaataacagcaggcggcaggactatcaggtacaattgtacctgtggccgtgacaacgtaggcactaccagtactgacaagaccatcaacacatgcaagattgaccaatgccatgctgccgttaccagccatgacaaatggcaatttacctctccatttgttcccagggctgatcagacagctaggaggggcaaagtgcatgttccattccctttgactaacgtcacctgccgagtgccgttggctcgagcgccggatgtcacctatggtaagaaggaggtgaccctgagattacacccagatcatccgacgctcttctcctataggagtttaggagccgaaccgcacccgtacgaggagtgggttgacaagttctctgagcgcatcatcccagtgacggaagaagggattgagtaccagtggggcaacaacccgccggtccgcctatgggcgcaactgacgaccgagggcaaaccccatggctggccacatgaaatcattcagtactattatggactataccccgccgccaccattgccgcagtatccggggcgagtctgatggccctcctaactctagcggccacatgctgcatgctggccaccgcgaggagaaagtgcctaacaccatacgccttgacgccaggagcggtggtaccgttgacactggggctgctttgctgcgcaccgagggcgaacgca

SEQ ID NO: 10 - protein, 16 aa, Marburg glycoprotein C-terminus mutation
LIALSCICRIFTKYIG

SEQ ID NO: 11 - protein, 16 aa, Marburg glycoprotein C-terminus mutation
LIALSAICRIFTKYIG

SEQ ID NO: 12 - protein, 16 aa, Marburg glycoprotein C-terminus mutation
LIALSCIARIFTKYIG

SEQ ID NO: 13 - protein, 16 aa, Marburg glycoprotein C-terminus mutation
LIALSSICRIFTKYIG

SEQ ID NO: 14 - protein, 16 aa, Marburg glycoprotein C-terminus mutation
LIALSCISRIFTKYIG

SEQ ID NO: 15 - protein, 13 aa, Marburg glycoprotein C-terminus mutation
LIALSCICRIFTK

SEQ ID NO: 16 - protein, 10 aa, Marburg glycoprotein C-terminus mutation
LIALSCICRK